(19) United States
(12) Patent Application Publication
(10) Pub. No.: US 2025/0160193 A1
(43) Pub. Date: May 15, 2025

(54) CONJUGATED POLYMERS AS HOLE TRANSPORT MATERIALS FOR ENHANCED THERMAL STABILITY IN OPTOELECTRONIC DEVICES

(71) Applicant: Georgia Tech Research Corporation, Atlanta, GA (US)

(72) Inventors: Juan-Pablo Correa-Baena, Atlanta, GA (US); Sanggyun Kim, Atlanta, GA (US); Carlo Andrea Riccardo Perini, Atlanta, GA (US); John Robert Reynolds, Dunwoody, GA (US); Sina Sabury, Atlanta, GA (US)

(21) Appl. No.: 18/944,564

(22) Filed: Nov. 12, 2024

Related U.S. Application Data
(60) Provisional application No. 63/597,853, filed on Nov. 10, 2023.

(51) Int. Cl. H10K 85/10; C08G 61/12; H10K 30/86

(52) U.S. Cl. CPC H10K 85/113; C08G 61/126; C08G 2261/124; C08G 2261/1424; C08G 2261/312; C08G 2261/3223; C08G 2261/3243; C08G 2261/3247; C08G 2261/512; C08G 2261/91; H10K 30/86

(57) ABSTRACT

The present disclosure provides polymers and oligomers having a constitutional repeating unit of Formula I. A composition including the disclosed polymer and oligomer is also provided. Devices including the disclosed polymer, oligomer, or composition are also provided. A photovoltaic device is provided including at least one perovskite material layer and at least one hole transport layer including the disclosed polymer, oligomer, or composition.

### CROSS-REFERENCE TO RELATED APPLICATIONS

This application claims the benefit of priority to U.S. Provisional Patent Application No. 63/597,853 filed Nov. 10, 2023, the disclosure of which is incorporated herein by reference in its entirety.

### STATEMENT REGARDING FEDERALLY SPONSORED RESEARCH OR DEVELOPMENT

This invention was made with government support under Grant No. EE0009524 awarded by the Department of Energy. The Government has certain rights in the invention.

### BACKGROUND

The transition away from nonrenewable fossil fuels to renewable alternative sources of energy has been an ongoing area of interest. One method comprises harvesting solar energy, such as through the use of perovskite solar cells (PSCs). While organic-inorganic hybrid PSCs have shown dramatic improvements in power conversion efficiency (PCE), they suffer from issues related to their long-term stability. Typically, PSCs suffer from degradation and thermal instability at the interface between the perovskite and the hole transport layer (HTL). While conjugated polymer (CP) HTLs have shown improvements, those previously explored have glass transition temperatures too close to the operating temperatures of PSCs, which pose challenges for long-term stability. Further, CPs cost twice as much as the components used in other HTLs.

There is a clear need for the development of PSCs containing HTLs with improved thermal stability while formed from components with reduced manufacturing costs. This disclosure addresses this as well as other needs.

### SUMMARY

The present disclosure is directed to polymers and oligomers, as well as compositions and devices formed from said polymers and oligomers. The disclosed polymers and oligomers may be used in devices such as optoelectronic devices, hole transport layers, or other uses for conducting polymers.

In one aspect, a polymer or oligomer is provided. In some aspects, the polymer or oligomer can comprise a constitutional repeating unit of Formula I

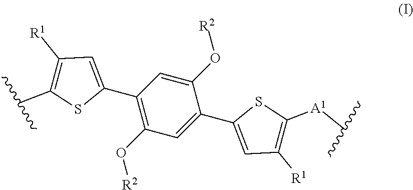

wherein all variables are as defined herein.

In another aspect, a composition is provided comprising a polymer or oligomer described herein.

In yet another aspect, a device is provided comprising a polymer, oligomer, or composition as described herein. In some aspects, the device can be a photovoltaic device, a light-emitting diode, or a field-effect transistor.

In yet another aspect, a photovoltaic device is provided. In some aspects, the photovoltaic device can comprise at least one perovskite material layer. In some aspects, the photovoltaic device can comprise at least one hole transport layer including a polymer, oligomer, or composition described herein.

The details of one or more aspects of the disclosure are set forth in the accompanying drawings and the description below. Other features, objects, and advantages of the disclosure will be apparent from the description, the drawings, and the claims.

### DESCRIPTION OF DRAWINGS

FIGS. 1A-1B depict the molecular structure and DSC curves of newly synthesized thiophene-based conjugated polymer HTLs. (FIG. 1A) Molecular structures and (FIG. 1B) DSC scans of TPT-TT, TPT-T, and TPT-T (MB/C6). The indicated portion of each molecular structure indicates where modifications are made to improve the thermal stability of each conjugated polymer. The arrows on the DSC data represent different thermal transition points. The heating rate was 10° C./min in N₂ atmosphere.

FIGS. 2A-2B depict the optoelectronic properties of TPT-based conjugated polymers. (FIG. 2A) Normalized UV-vis absorption spectra of TPT-TT, TPT-T, and TPT-T (MB/C6) as thin films on FTO substrates. (FIG. 2B) Energy level schematic of the halide perovskite, the TPT-based polymers, and the metal contact. The red dashed line represents the Fermi level energy of each material.

FIGS. 3A-3F depict the photovoltaic performance of TPT-based conjugated polymer HTLs in PSCs. (FIG. 3A) Device configuration of the n-i-p PSC. (FIG. 3B) J_{sc}, (FIG. 3C) V_{oc}, (FIG. 3D) FF, and (FIG. 3E) PCE obtained from reverse J-V scans. (FIG. 3F) Long-term stability of PSCs under constant simulated AM 1.5G illumination and MPPT for 200 h with continuous N₂ flow at 65° C.

FIGS. 4A-4B depict the chemical composition and structural characterization of PSCs before and after long-term stability test. (FIG. 4A) Pb 4f and I 3d XPS spectra and (FIG. 4B) 1D integrated GIWAXS patterns on TPT-TT, TPT-T, and TPT-T (MB/C6) surfaces on completed PSCs. GIWAXS data were obtained with a grazing incidence angle of 0.1°.

FIGS. 5A-5F depict the photovoltaic performance of PSCs on TPT-T (MB/C6) with Li-TFSI and tBP additives. (FIG. 5A) Energy level schema of TPT-T (MB/C6) and doped TPT-T (MB/C6). The red dashed lines represent the Fermi level energy of each material. (FIG. 5B) J_{sc}, (FIG. 5C) V_{oc}, (FIG. 5D) FF, (FIG. 5E) PCE, and (FIG. 5F) long-term stability of PSCs under constant simulated AM 1.5G illumination and MPPT for 200 h with continuous N₂ flow at 65° C. “Li-TFSI” refers to a combination of Li-TFSI and tBP additives.

FIG. 6 depicts the general synthesis scheme for the three TPT-based conjugated polymers, TPT-TT (R₁: Octyl, R₂: Decyl), TPT-T (R₁: Octyl, R₂: Decyl), and TPT-T(MB/C6) (R1: 2-Methyl butyl, R₂: Hexyl).

FIG. 7 depicts the GPC trace of the TPT-based polymers, TPT-TT (Mn=15 kg/mol, Ð: 1.64), TPT-T (Mn=24 kg/mol, Ð: 1.51), and TPT-T(MB/C6) (Mn=26 kg/mol, Ð: 2.3) using 1,2,4-trichlorobenzene as solvent at high temperature (140° C.).

FIGS. 8A-8C depict DSC thermograms of (FIG. 8A) TPT-TT, (FIG. 8B) TPT-T, and (FIG. 8C) TPT-T (MB/C6) measured at a scan rate of 10° C./min in N₂ atmosphere.

FIG. 9A depicts the UV-Vis absorption spectra of TPT-based conjugated polymer thin films. FIG. 9B depicts the optical energy band gap of CsFA perovskite and HTL thin films used in Example 1 via Tauc method.

FIGS. 10A-10F depict the UPS spectrum, secondary electron cut-off (SECO), and valence band onset of (FIG. 10A) CsFA perovskite, (FIG. 10B) Spiro-OMeTAD, (FIG. 10C) Doped Spiro-OMeTAD, (FIG. 10D) TPT-TT, (FIG. 10E) TPT-T, and (FIG. 10F) TPT-T (MB/C6).

FIG. 11 depicts the energy level of CsFA perovskite and different HTL thin films. The lower dashed line represents the Fermi level energy of each material.

FIGS. 12A-12F depict the statistics of (FIG. 12A) Jsc, (FIG. 12B) VOC, (FIG. 12C) FF, (FIG. 12D) PCE, (FIG. 12E) stabilized PCE, and (FIG. 12F) J-V curves of champion devices for REF and TPT-based conjugated polymers incorporated PSCs. REF refers to “doped Spiro-OMeTAD” used for control.

FIG. 13 depicts the evolution of the J-V parameters during long-term stability measurement of REF, TPT-TT, TPT-T, and TPT-T (MB/C6). Photovoltaic parameters from J-V scans were automatically extracted every 12 hours during the MPPT at 65° C. REF refers to “doped Spiro-OMeTAD” used for control.

FIGS. 14A-14B depict (FIG. 14A) Top-view SEM and (FIG. 14B) OM images of pristine and thermally stressed REF, TPT-TT, TPT-T, and TPT-T (MB/C6) on completed PSCs. For SEM, no obvious change in morphology was found for TPT-based polymers due to their transparency. REF refers to “doped Spiro-OMeTAD” used for control.

FIGS. 15A-15B depict 1D integrated GIWAXS profiles of REF, TPT-TT, TPT-T, and TPT-T (MB/C6) (FIG. 15A) on completed PSCs before and after long-term stability measurement and (FIG. 15B) on FTO substrates before and after annealing at 100° C. for 20 min. REF refers to “doped Spiro-OMeTAD” used for control.

FIGS. 16A-16F depict the statistics of (FIG. 16A) Jsc, (FIG. 16B) VOC, (FIG. 16C) FF, (FIG. 16D) PCE, (FIG. 16E) stabilized PCE, and (FIG. 16F) J-V curves of champion devices for REF, TPT-T (MB/C6), and doped TPT-T (MB/C6) incorporated PSCs. REF and 1.2M Li-TFSI refers to “doped Spiro-OMeTAD” used for control and combination of Li-TFSI and tBP additives, respectively.

FIG. 17 depicts the evolution of the J-V parameters during long-term stability measurement of REF, TPT-T (MB/C6), and doped TPT-T (MB/C6). Photovoltaic parameters from J-V scans were automatically extracted every 12 hours during the MPPT at 65° C. REF and 1.2M Li-TFSI refers to “doped Spiro-OMeTAD” used for control and combination of Li-TFSI and tBP additives, respectively.

FIGS. 18A-18D depict: the structure of PEDOT (FIG. 18A), PBTTT (FIG. 18B), and TPT-TT (FIG. 18C) polymers; a schematic representation of non-covalent interactions contributing to chain conformations in solution and solid states (FIG. 18D).

FIGS. 19A-19B depict: (FIG. 19A) Torsional potentials around the phenylene-thienylene (P-T) and thienylene-thienothiophene (T-TT) groups; the most stable conformers for both T-TT and P-T dihedrals are shown in the repeat unit structure. (FIG. 19B) Time-dependent evolution of the radius of gyration of the TPT-TT chain, as obtained from molecular dynamics simulations of the transformation of a linear rod TPT-TT polymer chain to stacked-rod and toroid structures.

FIGS. 20A-20D depict: Photographs (FIG. 20A) and spectra (FIG. 20B) of room-temperature 0.1 mg mL⁻¹ TPT-TT solutions in chlorobenzene (CB, dotted line) and toluene (Tol, solid line), as well as a thin film UV-vis spectrum (dashed line). (FIG. 20C) and (FIG. 20D) Thermochromism experiment from 10-60° C. demonstrating the temperature-dependent solution aggregation behavior of TPT-TT in toluene at a concentration of 0.1 mg mL-1.

FIG. 21 depicts the DSC profile with multiple thermal transitions and phases for the TPT-TT powder along with CPOM images (white scale bars=20 μm.) of thin films at different temperatures during the cooling cycle which was performed after the heating the sample to 250° C. Dark CPOM image at 230° C. shows isotropy at temperatures above highest thermal transition while brightness and patterns of CPOM images during the cooling process indicates the solidification through ordered intermediate phases.

FIGS. 22A-22C depict identification of the nematic phase in the first cooling cycle (215° C. to 125° C.). (FIG. 22A) In situ GIXD images at various temperatures. (FIG. 22B) In-plane and out-of-plane linecuts (left two); π-stacking peaks extracted by subtracting the 215° C. amorphous peak at each temperature (middle); calculated lamellar and π-stacking distances (right). (FIG. 22C) CPOM images of TPT-TT liquid crystal samples with schematic illustration of the nematic phase structure.

FIGS. 23A-23F depict identification of Smectic A phase in the first cooling cycle (125° C. to 50° C.). (FIG. 23A) In situ GIXD images across temperatures. (FIG. 23B) Linecut along q_{z} at q_{xy}=1.35 Å⁻¹ revealing (020), (030) π-stacking peaks at 0.70 Å⁻¹ and 1.05 Å⁻¹, respectively. Out-of-plane (χ=0°) (FIG. 23C) and χ=40° (FIG. 23D) linecuts revealing smectic layer and side-chain tilt peaks. (FIG. 23E) Linecuts along azimuthal angle at qᵣ=1.48 Å⁻¹ for tilted peaks 1. (FIG. 23F) Proposed schematic of smectic A phase structure.

FIGS. 24A-24E depict CPOM (FIG. 24A) and (FIG. 24B) and GIXD (FIG. 24C) and (FIG. 24D) images for as-cast (FIGS. 24A, 24C) and annealed (FIGS. 24B, 24D) thin-films and the schematic representation of the observed chain ordering and side-chain interdigitation upon annealing (FIG. 24E). The thin films are annealed by heating to 250° C. followed by a slow cooling (˜10° C. min⁻¹) to room temperature, similar to the DSC and CPOM experiments. The GIXD of the annealed films are obtained at room temperature.

FIGS. 25A-25C depict the electrical properties of TPT-TT transistors. (FIG. 25A) Transfer curve in the saturation regime (V_{DS}=−50 V) for a device that was annealed prior to testing, having L=30 μm and W=400 μm. The drain current is plotted on a log scale in black (left axis), while in blue we show the square root of the current (right axis). The inset shows the device structure. (FIG. 25B) Output curves for the same device. (FIG. 25C) Mobility values for the OFETs fabricated with as-cast (blue) and annealed (orange) films.

FIG. 26 depicts the dihedral scan of phenylenethienylene and thienylene-thienothiophene using optimized OPLS-AA Force Field and comparison with DFT Results.

FIG. 27 depicts temperature-dependent TPT-TT trimer chain separation in chlorobenzene and toluene.

FIG. 28 depicts the synthesis pathway of TPT-TT. Yields are shown in parentheses.

FIG. 29 depicts the ¹H NMR of TPT-TT in CDCl₃ at room temperature.

FIG. 30 depicts the high temperature (140° C.) GPC trace of TPT-TT dissolved in trichlorobenzene (Mn=24.6 kg/mol, Ð=1.93).

FIG. 31 depicts the MALDI-TOF spectrum of TPT-TT. The mass to charge (m/z) peaks are separated by the mass of the TPT-TT unit.

FIG. 32 depicts end group analysis for TPT-TT in the mass to charge (m/z) range of 4400-5000 based on the MALDI-TOF spectrum. Identified mass to charge numbers show that chains are mostly terminated by HBr end groups followed by HH and BrBr for TPT₅-TT₅, respectively. TPT:TT incorporation is considered equivalent. Deviation from 1:1 in observed species of TPT₅-TT₄ and TPT5-TT6 are related to the end units. Higher offset would be indicative of possible homocoupling which is not observed here.

FIG. 33 depicts computationally estimated UV-vis absorption spectra of TPT-TT in (a) toluene and (b) chlorobenzene. The main absorption feature is determined to be predominantly due to the S₀→S₁ electronic transition at λ=482 nm (2.56 eV).

FIG. 34 depicts thermogravimetric analysis of TPT-TT ramping from 50° C. to 800° C. at a rate of 20° C./min under nitrogen environment. TPT-TT is thermally stable, and the onset of the decomposition process (405° C. at 5% weight loss) is well above the thermal transitions observed in the DSC.

FIG. 35 depicts the heating and cooling processes of TPT-TT powder sandwiched between two glass slides. The powder at 25° C. was heated to form a melt at 220° C. (first row). Cross-polarized optical images (CPOM) show the emergence of liquid crystalline phase upon cooling from the melt state (second row). CPOM images show the heating process in the second thermal cycle resulting in an isotropic phase at 220° C. (third row).

FIGS. 36A-36B depict in-situ GIXD (FIG. 36A) and CPOM (FIG. 36B) images and of as-spun film during the first heating cycle.

FIG. 37 depicts GIXD analysis and temperature-dependent structural changes. a) Representative GIXD image with linecut locations. b) Out-of-plane linecut at a 90° azimuthal angle across different temperatures. c) Variation of lamellar stacking distance and peak intensity with heating temperature. d) Linecut at a 35° azimuthal angle across different temperatures. e) Variation of π-π stacking distance and peak intensity with heating temperature.

FIG. 38 depicts in-situ GIXD images during first cooling cycle at 35° C. and 25° C.

FIG. 39 depicts the indexing of the diffraction peaks from GIXD of the annealed films with the labeled Bragg rods (left) and full simulated diffraction pattern (right) with a P1 symmetry to show all possible peak positions.

FIG. 40 depicts (a) cyclic voltammetry (solid lines) and differential pulse voltammetry (dashed lines) of TPT-TT measured in degassed 0.5 M TBAPF6/propylene carbonate. The films were prepared by drop casting from 2 mg/mL chloroform solutions onto glassy carbon electrodes. The cyclic voltammograms were recorded at 20 mV/s whereas DPV was measured with a step size of 2 mV, a step time of 0.08 seconds, and a pulse amplitude of 20 mV from −0.58 to 0.72 eV vs. Fc/Fc+. (b) Potential dependent spectra of a TPT-TT film on ITO/glass in 0.1 V increments, demonstrating the bleaching of the π-π* of the neutral polymer as the film is electrochemically oxidized.

FIG. 41 depicts UV-vis-NIR spectra obtained during chemical doping of TPT-TT solution (0.05 mg/mL in CHCl₃) with 10 mg/mL solution of iron(III) p-toluenesulfonate (ferric tosylate) in acetonitrile added in 0.25 equiv. (relative to repeat unit) increments from 0 to 2 eq, then +1 equiv. until 4 equiv. where the depletion of the π-π* band is an indication of successful conversion of the neutral to the doped state.

FIG. 42 depicts electrical conductivity and Seebeck coefficient for as-cast and annealed TPT-TT films. Each individual point represents a measured film. Averages and sample standard deviations are annotated on the graph.

FIG. 43 depicts the electrical properties of TPT-TT transistors that have not been annealed. a) Transfer curve in the saturation regime (VDS=−50 V) for a device that was annealed prior to testing, having L=40 μm and W=1000 μm. b) Output curves for the same device.

FIG. 44 depicts J-V curves and mobility fits (a), and thickness dependence checks (b) for un-annealed samples. Data for the annealed samples are shown in (c, J-V curves and mobility fits) and (d, thickness dependence checks) in SCLC regime hole-only mobility measurements for TPT-TT.

FIGS. 45A-45B depict the incorporation of EDOT as a pi-bridge in TPT systems via the synthesis of TPT-E (FIG. 45A) and TPT-2E (FIG. 45B).

FIGS. 46A-46B depict the induction of solution aggregation in TPT-E (FIG. 46A) and TPT-2E (FIG. 46B).

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning the arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, “consisting essentially of” is intended to include examples encompassed by the term “consisting of.”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

The terms “coupled” and “associated” generally mean electrically, electromagnetically, and/or physically (e.g., mechanically or chemically) coupled or linked and do not exclude the presence of intermediate elements between the coupled or associated items.

It will be understood that when an element is referred to as being “connected” or “coupled” to another element, it can be directly connected or coupled to the other element, or intervening elements can be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

It will be understood that although the terms “first,” “second,” etc., can be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

Spatially relative terms, such as “beneath,” “below,” “lower,” “above,” “upper,” “upward,” “downward,” “top,” “bottom,” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device can be otherwise oriented (rotated 90 degrees or at other orientations), and the spatially relative descriptors used herein are interpreted accordingly.

Terms such as “proximal,” “distal,” “radially outward,” “radially inward,” “outer,” “inner,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar import. Similarly, the terms “first,” “second,” and other such numerical terms referring to structures neither imply a sequence nor order unless clearly indicated by the context.

As used herein, the term “substantially” means that the subsequently described event or circumstance completely occurs or that the subsequently described event or circumstance generally, typically, or approximately occurs.

Still further, the term “substantially” can, in some aspects, refer to at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R) or (S) configuration. The compounds provided herein may either be enantiomerically pure or be diastereomeric or enantiomeric mixtures. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Compounds described herein may contain one or more double bonds and, thus, potentially give rise to cis/trans (E/Z) isomers, as well as other conformational isomers. Unless stated to the contrary, all such possible isomers are contemplated, as well as mixtures of such isomers.

Compounds described herein may also present as an equilibrium of tautomers. For example, ketones with an α-hydrogen can exist in an equilibrium of the keto form and the enol form. Likewise, amides with an N-hydrogen can exist in an equilibrium of the amide form and the imidic acid form. Unless stated to the contrary, all possible tautomers of the compounds described herein are contemplated.

A dash (“-”) that is not between two letters or symbols is used to indicate a point of attachment for a substituent. For example, —(C═O)NH₂ is attached through the carbon of the keto (C═O) group.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., ═O), two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and/or can be formulated into a form with a shelf life of at least one month. A stable manufacturing intermediate or precursor to an active compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: halo, nitro, cyano, azido, oxo, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, A^{x}O—(C₀-C₆ alkyl)-, A^{x}S—(C₀-C₆ alkyl)-, (A^{x} Å^{y}N)—(C₀-C₆ alkyl)-, A^{z}C(O)—(C₀-C₆ alkyl)-, A^{z}C(N)—(C₀-C₆ alkyl)-, and A^{z}S(O)—(C₀-C₆ alkyl)-, and A^{z}S(O)₂—(C₀-C₆ alkyl)-, wherein A^{x} and A^{y} are independently selected at each occurrence from A^{a}, A^{z}C(O)—, A^{z}C(N)—, A^{z}S(O)—, and A^{z}S(O)₂—, each of which may be optionally substituted with one or more B groups as allowed by valency; wherein A^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OA^{a}, —SA^{a}, and —NA^{a}A^{b}, each of which may be optionally substituted with one or more B groups as allowed by valency; wherein A^{a} and A^{b} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted by one or more B groups as allowed by valency; and wherein B is independently selected at each occurrence from hydrogen, halo, nitro, cyano, azido, oxo, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, A^{p}O—, A^{p}S—, A^{p}A^{q}N—, A^{o}C(O)—, A^{o}C(O)—O—, A^{o}C(O)—NA^{q}-, A^{o}S(O)₂—, A^{o}S(O)₂—O—, and A^{o}S(O)₂—NA^{q}-, wherein A^{o} is independently selected at each occurrence from A^{p}, halo, A^{p}O—, and A^{p}A^{q}N—, and wherein A^{p} and A^{q} are independently selected at each occurrence from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, and (5- to 10-membered monocyclic or bicyclic heteroaryl)-(C₀-C₆ alkyl)-.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person in the context in which said functional groups are recited.

As used herein, the symbol

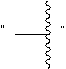

(which hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

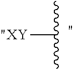

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

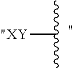

infers that when R³ is “XY”, the point of attachment bond is the same bond as the bond by which R³ is depicted as being bonded to CH₃.

“Halo” or “halogen” indicates, independently, any of fluoro, chloro, bromo or iodo.

The term “nitro” as used herein is represented by the formula —NO₂.

The term “cyano” as used herein is represented by the formula —CN

The term “azido” as used herein is represented by the formula —N₃.

The term “oxo” as used herein is represented by the formula ═O.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some aspects, the alkyl group is optionally substituted as described herein.

“Haloalkyl” refers to an alkyl group that is substituted with one or more halo groups, e.g., fluoro, chloro, bromo, iodo, or combinations thereof.

“Cycloalkyl” is a saturated or partially unsaturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some aspects, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms. In one aspect, the heterocycle group is optionally substituted as described herein.

“Heteroaryl” refers to a stable monocyclic, bicyclic, or multicyclic aromatic ring which contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. In one aspect, the heteroaryl group is optionally substituted as described herein.

As used herein, a “polymer” refers to a substance composed of macromolecules, i.e., molecules of high relative mass, the structure of which essentially comprises the multiple repetitions of units derived, actually or conceptually, from molecules of low relative molecular mass.

As used herein, an “oligomer” or “oligomer molecule” refers to a molecule of intermediate relative molecular mass, the structure of which essentially comprises a small plurality of units derived, actually or conceptually, from molecules of lower relative molecular mass.

As used herein, a “constitutional unit” refers to an atom or group of atoms (with pendant atoms or groups, if any) comprising part of the essential structure of a macromolecule (e.g., a polymer), an oligomer molecule, a block or a chain. As used herein, a “constitutional repeating unit” refers to the smallest constitutional unit, the repetition of which constitutes a regular macromolecule, a regular oligomer molecule, a regular block, or a regular chain.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers, such as Sigma-Aldrich (formally MilliporeSigma, Burlington, MA) or Thermo Fisher Scientific Inc. (Waltham, MA), or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8^{th} Edition); and Larock's Comprehensive Organic Transformations (John Wiley and Sons, 3^{rd} edition, 2017).

### Compositions

In one aspect, a polymer or oligomer is provided comprising a constitutional repeating unit of Formula I

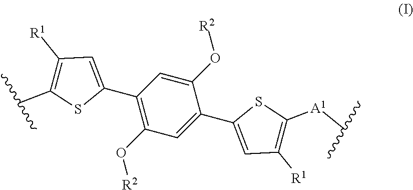

wherein:
R¹ and R² are independently selected at each occurrence from C₁-C₂₀ hydrocarbyl and —(CH₂CH₂O)ₘOCH₂CH₃, wherein m is an integer selected from 1 to 4, wherein each R¹ and R² may be optionally substituted with one or more groups independently selected from halogen, —NR^{a}R^{b}, —OR^{a}, and —P(═O)(OR^{a})(OR^{b});
R^{a} and R^{b} are independently selected at each occurrence from hydrogen and C₁-C₂₀ hydrocarbyl; and
A¹ is selected from:

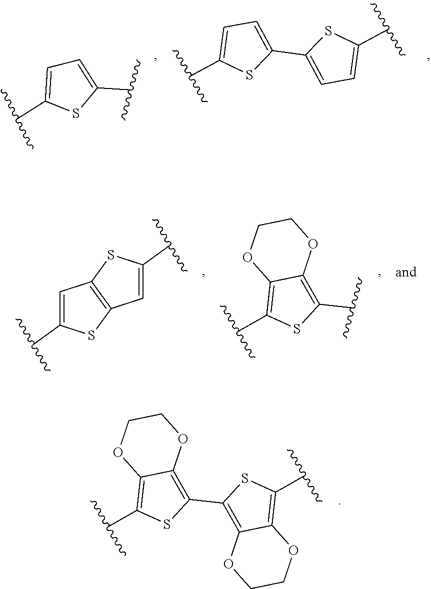 [SMILES: *c1ccc(*)s1 ; *c1ccc(-c2ccc(*)s2)s1 ; *c1cc2sc(*)cc2s1 ; *c1sc(*)c2c1OCCO2 ; *c1sc(-c2sc(*)c3c2OCCO3)c2c1OCCO2]

As used herein, “C₁-C₂₀ hydrocarbyl” and the like generally refer to aliphatic, aryl, or arylalkyl groups containing 1 to 20 carbon atoms. Examples of aliphatic groups, in each instance, include, but are not limited to, an alkyl group, a cycloalkyl group, an alkenyl group, a cycloalkenyl group, an alkynyl group, an alkadienyl group, a cyclic group, and the like, and includes all substituted, unsubstituted, branched, and linear analogs or derivatives thereof, in each instance having 1 to about 20 carbon atoms. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl. Examples of aryl or arylalkyl moieties include, but are not limited to, anthracenyl, azulenyl, biphenyl, fluorenyl, indan, indenyl, naphthyl, phenanthrenyl, phenyl, 1,2,3,4-tetrahydro-naphthalene, tolyl, xylyl, mesityl, benzyl, and the like, including any heteroatom substituted derivative thereof. In some aspects, the hydrocarbyl group is optionally substituted as described herein.

In some aspects, R¹ is independently selected at each occurrence from C₁-C₂₀ hydrocarbyl optionally substituted with one or more (e.g., 1, 2, 3, or 4) groups independently selected from halogen, —NR^{a}R^{b}, —OR^{a}, and —P(═O)(OR^{a})(OR^{b}). In some aspects, R¹ is independently selected from C₁-C₂₀ alkyl. In some aspects, R¹ is independently selected from methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. In some aspects, R¹ is —(CH₂)₅CH₃. In some aspects, R¹ is —(CH₂)₉CH₃. In some aspects, R¹ is independently selected from branched C₁-C₂₀ alkyl, such as —CH₂(CHCH₃)CH₂CH₃.

In some aspects, R¹ is independently selected at each occurrence from —(CH₂CH₂O)ₘOCH₂CH₃, wherein m is an integer selected from 1 to 4, optionally substituted with one or more (e.g., 1, 2, 3, or 4) groups independently selected from halogen, —NR^{a}R^{b}, —OR^{a}, and —P(═O)(OR^{a})(OR^{b}). In some aspects, m is 1. In some aspects, m is 2. In some aspects, m is 3. In some aspects, m is 4.

R¹ and R² are independently selected at each occurrence from C₁-C₂₀ hydrocarbyl and —(CH₂CH₂O)ₘOCH₂CH₃, wherein m is an integer selected from 1 to 4, wherein each R¹ and R² may be optionally substituted with one or more groups independently selected from halogen, —NR^{a}R^{b}, —OR^{a}, and —P(═O)(OR^{a})(OR^{b});

In some aspects, R² is independently selected at each occurrence from C₁-C₂₀ hydrocarbyl optionally substituted with one or more (e.g., 1, 2, 3, or 4) groups independently selected from halogen, —NR^{a}R^{b}, —OR^{a}, and —P(═O)(OR^{a})(OR^{b}). In some aspects, R² is independently selected from C₁-C₂₀ alkyl. In some aspects, R² is independently selected from methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. In some aspects, R² is —(CH₂)₇CH₃. In some aspects, R² is independently selected from branched C₁-C₂₀ alkyl, such as —CH₂(CHCH₃)CH₂CH₃. In some aspects, R² is —CH₂(CHCH₃)CH₂CH₃.

In some aspects, R¹ is independently selected at each occurrence from —(CH₂CH₂O)ₘOCH₂CH₃, wherein m is an integer selected from 1 to 4, optionally substituted with one or more (e.g., 1, 2, 3, or 4) groups independently selected from halogen, —NR^{a}R^{b}, —OR^{a}, and —P(═O)(OR^{a})(OR^{b}). In some aspects, m is 1. In some aspects, m is 2. In some aspects, m is 3. In some aspects, m is 4.

In some aspects, the constitutional repeating unit of Formula I is selected from:

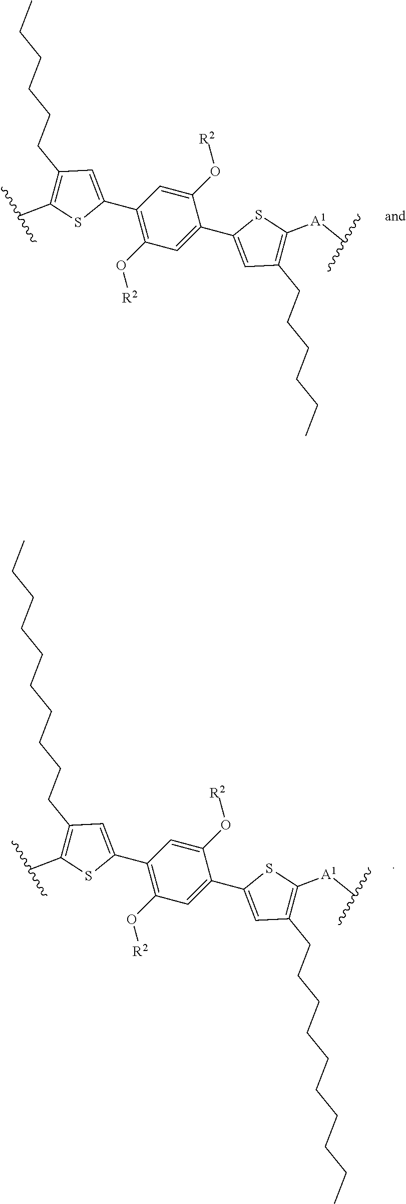

In some aspects, the constitutional repeating unit of Formula I is selected from:

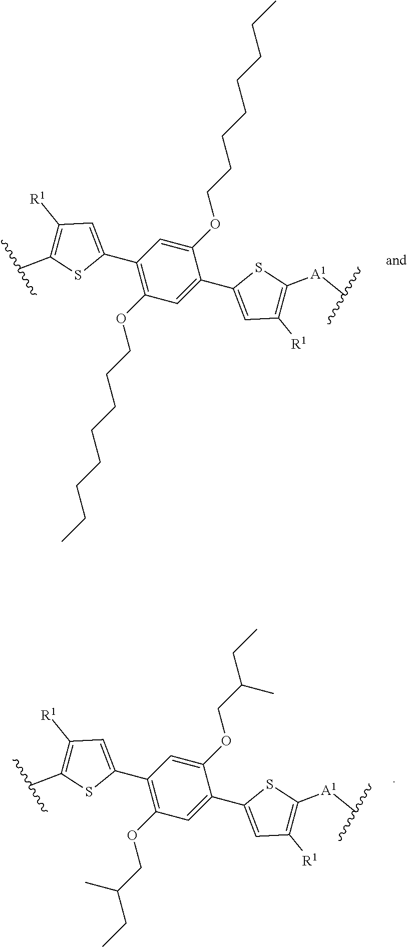

In some aspects, A¹ is

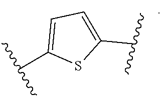 [SMILES: *c1ccc(*)s1]

In some aspects, A¹ is

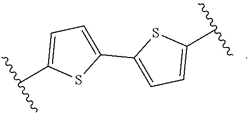 [SMILES: *c1ccc(-c2ccc(*)s2)s1]

In some aspects, A¹ is

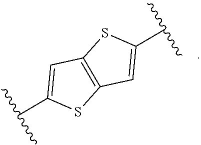 [SMILES: *c1cc2sc(*)cc2s1]

In some aspects, A¹ is

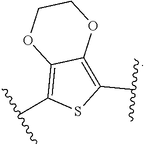 [SMILES: *c1sc(*)c2c1OCCO2]

In some aspects, A¹ is

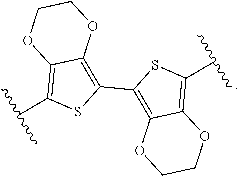 [SMILES: *c1sc(-c2sc(*)c3c2OCCO3)c2c1OCCO2]

In some aspects, the polymer or oligomer comprises a constitutional repeating unit comprising:

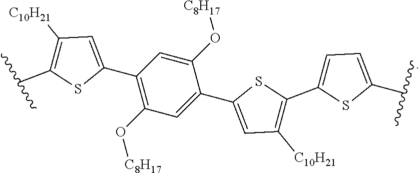

In some aspects, the polymer or oligomer comprises a constitutional repeating unit comprising:

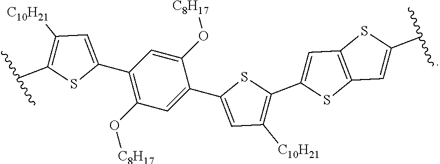

In some aspects, the polymer or oligomer comprises a constitutional repeating unit comprising:

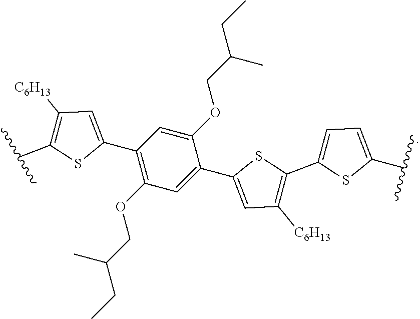

In some aspects, the polymer or oligomer comprises a constitutional repeating unit comprising:

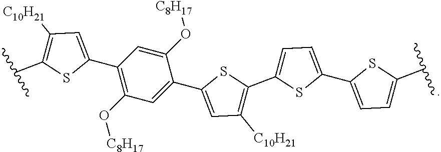

In some aspects, the polymer or oligomer comprises a constitutional repeating unit comprising:

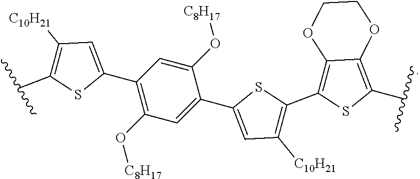

In some aspects, the polymer or oligomer comprises a constitutional repeating unit comprising:

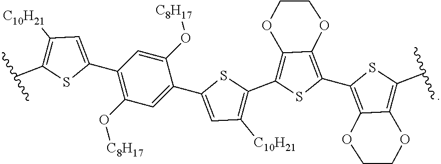

In another aspect, a composition is provided comprising a polymer or oligomer as described herein. In some aspects, the composition may comprise one or more additional additives, for example HMI-TFSI (1-hexyl-3-methylimidazolium bis(trifluoromethylsulfonyl)imide), Li-TFSI (lithium bis(trifluoromethylsulfonyl)imide), or tBP (tert-butylpyridine).

In some aspects, the polymer, oligomer, or composition described herein comprises a conducting material.

### Devices

In another aspect, a device is provided comprising a polymer, oligomer, or composition as described herein. In some aspects, the device comprises an optoelectronic device. In some aspects, the device comprises a photovoltaic device. In other aspects, the device comprises a light-emitting diode. In other aspects, the device comprises a field-effect transistor.

In some aspects, a photovoltaic device is provided comprising one or more hole transfer layers comprising a polymer, oligomer, or composition described herein. In some aspects, the photovoltaic further comprises one or more perovskite material layers. In some aspects, a hole transfer layer overlays a perovskite material layer.

In some aspects, the perovskite material layer comprises a perovskite material as known in the art. The term “perovskite,” as used herein, refers to a material with a three-dimensional crystal structure related to that of CaTiO₃ or a material comprising a layer of material, wherein the layer has a structure related to that of CaTiO₃. The structure of CaTiO₃ can be represented by the formula ABX₃, wherein A and B are cations of different sizes, and X is an anion. In the unit cell, the A cations are at (0,0,0), the B cations are at (1/2, 1/2, 1/2), and the X anions are at (1/2, 1/2, 0). The A cation is usually larger than the B cation. One of ordinary skill in the art will appreciate that when A, B, and X are varied, the different ion sizes may cause the structure of the perovskite material to distort away from the structure adopted by CaTiO₃ to a lower-symmetry distorted structure. The symmetry will also be lower if the material comprises a layer that has a structure related to that of CaTiO₃. Materials comprising a layer of perovskite material are well known. For example, the structure of materials of materials adopting the K₂NiF₄-type structure comprises a layer of perovskite material.

In some aspects, the perovskite material may be of the general formula ABX₃, wherein A comprises one or more monovalent cations, B comprises one or more divalent inorganic cations, and X comprises one or more anions.

In some aspects, the one or more monovalent cations A may comprise an organic cation, for example, methylammonium (MA), ethylammonium (EA), or formamidimium (FA). In some aspects, the one or more monovalent cations A may comprise an inorganic cation, for example, Cs⁺, Rb⁺, Cu⁺, Pd⁺, Pt⁺, Ag⁺, Au⁺, Rh⁺, and Ru⁺. In some aspects, the one or more monovalent cations A may comprise a combination of two or more of the above-listed organic cations and/or inorganic cations.

In some aspects, the one or more divalent inorganic cations B is a divalent metal cation. In some aspects, the one or more divalent inorganic cations B comprises one or more cations selected from Ca²⁺, Sr²⁺, Cd²⁺, Cu²⁺, Ni²⁺, Mn²⁺, Fe²⁺, Co²⁺, Pd²⁺, Ge²⁺, Sn²⁺, Pb²⁺, Sn²⁺, Yb²⁺, and Eu²⁺. In some aspects, the one or more divalent cations are selected from Sn²⁺, Pb²⁺, and Cu²⁺. In some aspects, the one or more divalent cations are selected from Sn²⁺ and Pb²⁺.

In some aspects, the one or more anions X comprises at least one anion selected from halide anions and chalcogenide anions. The term “halide anion” typically refers to a fluoride anion, a chloride anion, a bromide anion, an iodide anion, or an astatide anion. The term “chalcogenide anion” refers to an oxide anion, a sulfide anion, a selenide anion, or a telluride anion. In some aspects the one or more anions X comprises two or more halide anions.

In some aspects, the perovskite material can be Cs_{X}FA_{y}MA_{z}Pb(IₐBr_{b})₃, CsₓFA_{y}MA_{z}Sn(IₐBr_{b})₃, CsₓFA_{y}MA_{z}Ge(IₐBr_{b})₃, or any combination thereof. In such exemplary aspects, 0≤x≤1, 0≤y≤1, 0≤z≤1, 0≤a≤1, and 0≤b≤1. In further and unlimiting aspects, the bromide in the perovskite material can be at least partially substituted with fluoride, chloride, or iodide, for example.

In some aspects, the photovoltaic device can comprise any additional layers that may be present in a photovoltaic device.

For example, the photovoltaic device may comprise one or more electron transport layers in some aspects. The electron transport layer may tune the crystallinity of the corresponding perovskite material layer for improved device performance. Examples of materials that may be used as electron transport layers include but are not limited to, SnO₂ (such as mesoporous SnO₂), KOH, TiOH, MgO, Al₂O₃, 1-benzyl-3-methylimidazolium, NiO₂, MoOₓ, VOₓ, and 1-butyl-3-methylimidazolium.

In some aspects, the photovoltaic device can further comprise one or more transparent layers. Transparent layers, as used in photovoltaic devices, are known in the art. In some aspects, the transparent layer is conductive. In some aspects, the transparent layer can comprise indium tin oxide (ITO), glass, fluoride-doped tin oxides (FTO), gallium, doped zinc oxide glass, niobium-doped titanium oxide glass, ZnO, other conductive polymers, indium doped zinc oxide (IZO), aluminum-doped zinc oxide (AZO), and the like.

In some aspects, the photovoltaic device can comprise one or more blocking layers. Blocking layers, as used in photovoltaic devices, are known in the art. In some aspects, the blocking layer can be positioned between a perovskite material layer and an electron transport layer to inhibit electron recombination. In some aspects, the blocking layer can comprise TiO₂, SnO₂, ZnO, CuO, NiO, or combinations thereof.

In some aspects, the photovoltaic device can comprise a passivation layer. In some aspects, the passivation layer can be disposed between the hole transport layer and the perovskite material layer. In some aspects, the passivation layer can comprise phenylethylammonium iodide, octylammonium iodide, butylammonium iodide, anthracene, 4-fluorobenzamide, D-4-tert-butylphenylalanine, tetrabutylammonium hexafluorophosphate, formamidinium bromide, iodopentafluorobenzene, fullerene, theophylline, 4-dimethylaminebenzoic acid, tribenzylphosphine oxide, 2-cyanoacrylate, pyridine, Zn porphyrin, Co porphyrin, Ni porphyrin, Cu porphyrin, H porphyrin, 4-tert-butyl-benzylammonium iodide, azetidinium iodide, o-(phenylene)di(ethylammonium) iodide, phenethylamine hydroiodate, benzylamine, n-butylamine hydroiodate, dioctylamine iodide, octylamine, phenethylamine, n-butylamine, ethylamine, or combinations thereof.

In some aspects, the device can comprise a stack of layers in the following order: a transparent layer; a charge transport layer (e.g., an electron transport layer or a hole transport layer); a perovskite material layer; and another charge transport layer (e.g., an electron transport layer or a hole transport layer). In some aspects, the charge transport layer or the other charge transport layer comprises a polymer, oligomer, or composition as described herein.

In some aspects, a region of the photovoltaic device comprising the electron transport layer, perovskite material layer, and hole transport layer may comprise what is deemed a “photoactive region” of the device. In order to provide a highly efficient photovoltaic device, the absorption of the photoactive region should ideally be maximized so as to generate an optimal amount of current. As a result, when using a perovskite material in the photovoltaic device, the thickness of the perovskite material layer in some aspects may be from about 300 nm to about 600 nm, in some aspects, to absorb most of the sun light across the visible spectrum. In particular, the perovskite material layer may be thicker than the absorption depth (which is defined as the thickness required to absorb 90% of the incident light of a given wavelength), which for perovskite materials may be above 100 nm if significant light absorption is required across the whole visible spectrum (from about 400 to about 800 nm). The use of a photoactive region of lower thickness could be detrimental to the performance of the device.

A number of aspects of the disclosure have been described. Nevertheless, it will be understood that various modifications may be made without departing from the spirit and scope of the disclosure. Accordingly, other aspects are within the scope of the following claims.

By way of non-limiting illustration, examples of certain aspects of the present disclosure are given below.

### EXAMPLES

The following examples are set forth below to illustrate the compounds, compositions, articles, devices, and methods claimed herein, along with associated methods and results according to the disclosed subject matter. These examples are not intended to be inclusive of all aspects of the subject matter disclosed herein, but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions, that can be used to optimize the product purity and yield obtained from the described process. Only reasonable and routine experimentation will be required to optimize such process conditions.

### Example 1. Enhancing Thermal Stability of Perovskite Solar Cells Through Thermal Transition and Thin Film Crystallization Engineering of Polymeric Hole Transport Layers

Organic hole transport layers (HTLs) have been known to be susceptible to thermal stress, leading to poor long-term stability in perovskite solar cells (PSCs). We synthesized three 2,5-dialkoxy-substituted, 1,4-bis(2-thienyl)phenylene (TPT)-based conjugated polymers (CPs) linked with thiophene-based (thiophene (T) and thienothiophene (TT)) comonomers and evaluated them as HTLs in n-i-p PSCs. TPT-T (MB/C6), which has branched 2-methylbutyl and linear hexyl (MB/C6) side chains, emerged as a promising HTL candidate, enabling power conversion efficiencies (PCEs) greater than 15%. In addition, PSCs with this HTL showed an improvement in long-term stability at elevated temperatures of 65° C. when compared to those with the state-of-art HTL, 2,2′,7,7′-tetrakis(N,N-p-dimethoxyphenylamino)-9,9′-spirobifluorene (spiro-OMeTAD). This improvement is ascribed to the lack of thermal transitions within the operational temperature range of PSCs for TPT-T (MB/C6), which is attributed to the relatively short branched side chains of this polymer. We propose that the elimination of thermal transitions below 200° C. leads to HTLs without cracking as-deposited and after conducting a stress test at 65° C., which can serve as a new design guideline for HTL development.

### Introduction

Organic-inorganic hybrid perovskite solar cells (PSCs) have made remarkable strides in power conversion efficiency (PCE) over the past decade, reaching an impressive 26.1% for a single junction device. (1) Despite the high PCE values, long-term operational stability remains a critical challenge for PSC commercialization. Key factors limiting the achievement of long-term stability in devices are the mechanical and chemical changes in the hole transport layer (HTL), which interfaces with the perovskite during operation and thermal cycling. (2) These solar cells need to withstand elevated temperatures (i.e., 65° C.) in order to pass key International Summit on Organic Photovoltaics Stability (ISOS) metrics. (3,4) Thus, strategies aimed at enhancing the stability of HTLs in PSCs, while simultaneously maintaining device efficiency, are highly desirable.

Currently, the small molecule 2,2′,7,7′-tetrakis(N,N-p-dimethoxyphenylamino)-9,9′-spirobifluorene (spiro-OMeTAD), processed from solution as a thin film, serves as the benchmark HTL. Typically, spiro-OMeTAD is combined with additives such as lithium bis(trifluoromethane)sulfonimide (Li-TFSI) and 4-tert-butylpyridine (tBP) to increase the conductivity of the HTL and enable high efficiencies. However, these additives have been shown to migrate toward the HTL interfaces and interact with the perovskite thin film or metal contacts, which leads to long-term stability challenges. (5,6) In addition to the requirement for additives, the doped spiro-OMeTAD has been shown to crystallize at low temperatures within the range of solar cell operation, leading to the formation of cracks and consequently deteriorating the device performance. (7-10) To suppress the crystallization of spiro-OMeTAD, researchers have been studying how the molecular engineering of pristine molecules and the incorporation of additives can allow for the control of their thermal transitions. (11)

Conjugated polymer (CP)-based HTLs with improved thermal stability have been investigated as alternatives to spiro-OMeTAD. CPs are attractive HTL candidates owing to their tunable physical and electrical properties, which can be tailored via alteration of the conjugated backbone and side chains. (12) Poly[bis(4-phenyl)(2,4,6-trimethylphenyl)amine](PTAA) is the most widely used HTL polymer in PSCs, deposited from solution as a thin film of about 20-50 nm in thickness. (13-15) PTAA has thermal transitions as low as 98° C., which is higher than the typical temperatures to which solar cells are exposed during thermal cycling. (16,17) These thermal transitions are important to the viability of devices, as they are suggested to be responsible for long-term stability under thermal stress. To date, promising thiophene-based CP HTLs have been reported, including poly[3-(4-carboxybutyl)thiophene-2,5-diyl](P3CT) and poly[3-(6-carboxyhexyl)thiophene-2,5-diyl](P3HT-COOH). (18,19) Thiophene-based polymers are some of the most notable systems in CP research, primarily attributed to their electron-rich nature and straightforward synthetic routes. (20,21) Both P3CT- and P3HT-COOH-incorporated PSCs show encouraging PCEs above 20%. However, there is little understanding of their thermal stability, as stress factors including light, electrical bias, and heat were not introduced simultaneously. For P3CT, thermal stability tests were performed at 85° C. under a N₂ atmosphere in dark conditions for 144 h without maximum power point tracking (MPPT). The PSC measured over time lost 20% of its original PCE. (18) Similarly for P3HT-COOH, performance measurements were conducted at 65° C. in a N₂ atmosphere in dark conditions without MPPT, where the PSC experienced a loss of 20% of its original PCE.

In this example, a family of 1,4-(2-thienyl)-2,5-dialkoxyphenylene (TPT) core units copolymerized with thienothiophene (TT) and thiophene (T) is explored as HTLs in PSCs. These three polymers, with their syntheses presented in FIG. 6, are referred to as TPT-TT, TPT-T, and TPT-T (MB/C6). The side chains of the TPT-TT and TPT-T polymers are similar (octyl and decyl pendant from phenyl and the flanking thiophenes), and the π-bridge between the TPT units changes from a fused ring TT to a less electron rich T. TPT-TT has been reported to exhibit a high degree of planarity, promoted by noncovalent intramolecular S—O and S—H—C Coulombic interactions and a high out-of-plane hole mobility of (2.43±0.01)×10⁻⁴ cm² V⁻¹ s⁻¹. (22) The third polymer (FIG. 6), TPT-T (MB/C6), bears a shorter and branched 2-methylbutyl side chain (attached to the phenyl) and linear hexyl side chains (attached to flanking thiophenes). We conducted differential scanning calorimetry (DSC) to probe the physical properties, such as thermal transitions, of the different polymers. The three polymers are deposited as HTL thin films in PSCs and tested under 1 sun conditions. (3) The PSCs with the TPT-T (MB/C6) polymer as the HTL (without any additives) exhibited a PCE greater than 12%. In addition, we conducted long-term thermal stability measurements following the ISOS protocols. (3) The TPT-T (MB/C6) shows improved long-term stability compared to TPT-TT and TPT-T. Furthermore, TPT-T (MB/C6) combined with Li-TFSI and tBP additives shows a higher PCE of over 15% and withstands 200 h at 65° C. without significant changes in efficiency.

### Physical Properties of TPT-Based Conjugated Polymers

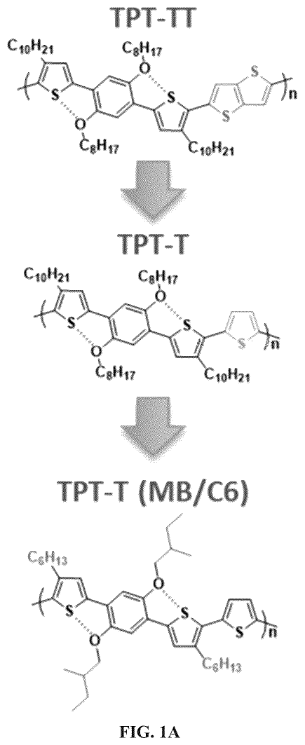

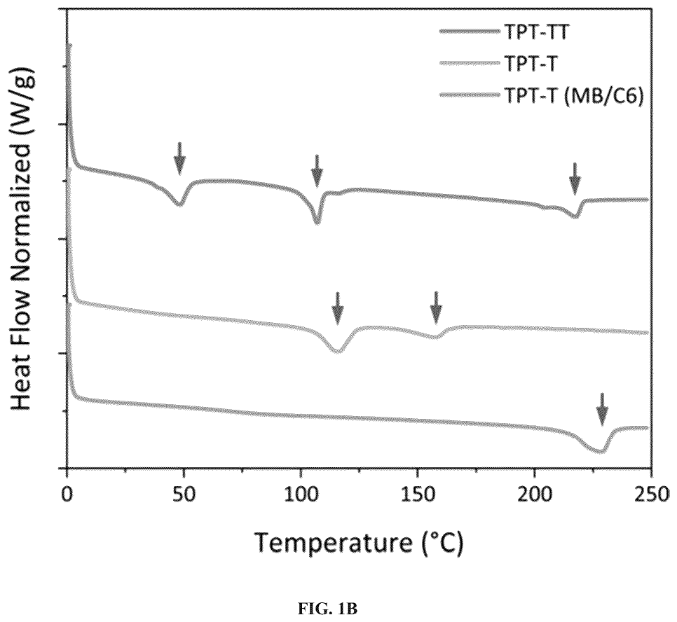

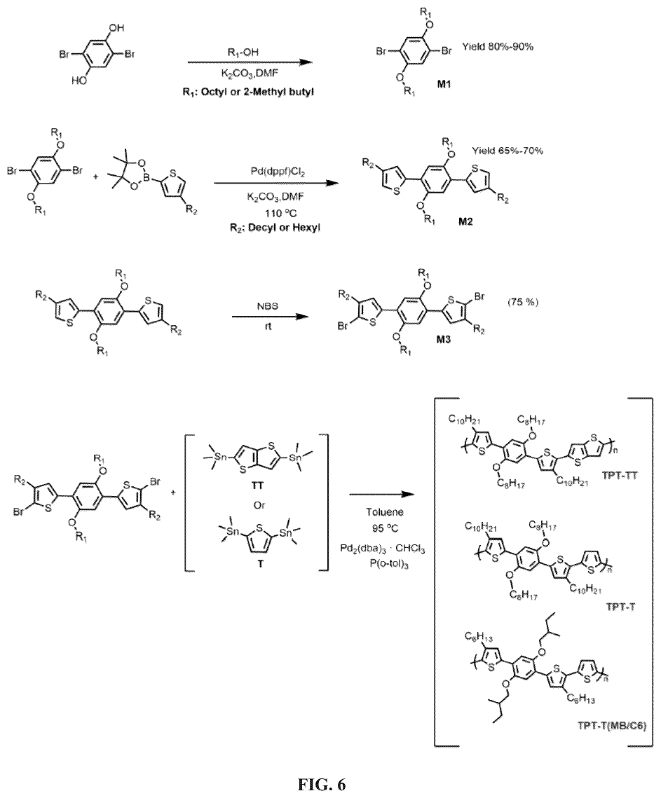

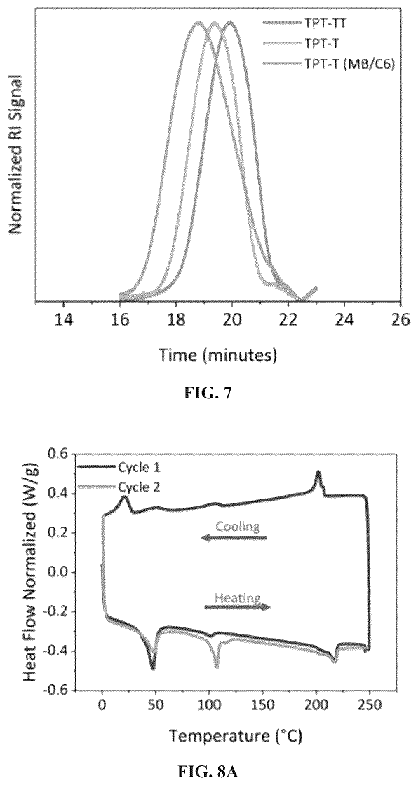

The molecular structures of TPT-TT, TPT-T, and TPT-T (MB/C6) are shown in FIG. 1A and are arranged in the sequence of their respective modifications (the synthesis route is shown in FIG. 6). The number-average molecular weights (Mₙ) and dispersity (Ð) of TPT-TT, TPT-T, and TPT-T (MB/C6) are 15 kg/mol (Ð 1.64), 24 kg/mol (Ð 1.51), and 26 kg/mol (Ð 2.30), respectively. M₁ and D values of the polymers were determined by high-temperature gel permeation chromatography (GPC) using 1,2,4-trichlorobenzene at 140° C. as the eluent. GPC traces are shown in FIG. 7 and are monomodal. The comparable M₁ and Ð values of TPT-T and TPT-T (MB/C6) suggest that any divergences between them are likely attributed to alterations in the backbone and side chain chemistry. The polymer purity was confirmed by elemental analysis and polymer structure using nuclear magnetic resonance (NMR) spectroscopy (Table 1). The detailed synthetic routes of the monomers and Stille cross-coupling polymerization to obtain TPT-based polymers are provided herein.

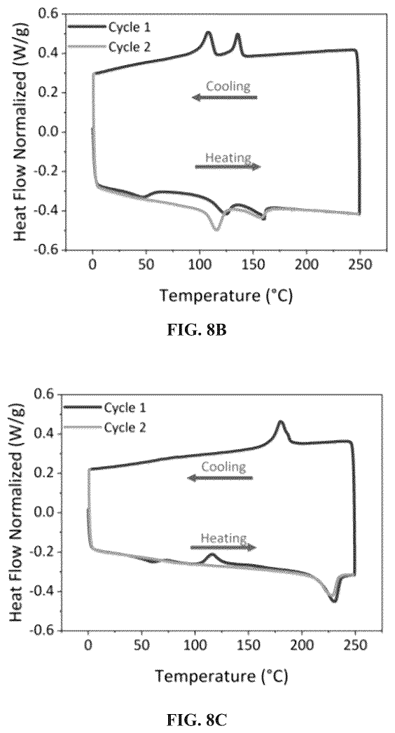

Examining the repeat unit structures in FIGS. 1A-1B, it can be seen that TPT-TT and TPT-T were designed and synthesized with linear octyloxy side chains on the phenylene unit and linear decyl groups on the thiophene unit. These conformationally flexible side chains provide increased solubility of the polymers for solution processing and, in this instance, lead to multiple thermal transitions, as evident from the DSC results (analyzed from the second heating scan to eliminate thermal history effects) shown in FIG. 1B. These types of transitions are expected when a polymer passes through liquid crystalline phases and, as will be discussed later, negatively affect its utility as an HTL. Distinct melting and crystallization features were present in all three polymers, confirming their semicrystalline nature (FIGS. 8A-8C). TPT-TT revealed three thermal transitions at 48° C., 107° C., and 218° C., where the lowest and the highest thermal transitions are due to the side chain and backbone order-disorder, while the transition at 107° C. is correlated with a liquid crystalline behavior. (22) Replacing a TT with T moves the first thermal transition (side chain order-disorder transition) to 116° C. (from 48° C. in TPT-TT), while the backbone melting drops to 157° C. (compared to 218° C.). These changes are in line with the improvement of the side chain packing strength (higher side chain melting point), while the backbone rigidity and π-π interactions might decrease due to modulation in intramolecular noncovalent interactions and removal of fused ring units from the repeat unit structure.

By shortening and using branched side chains in TPT-T (MB/C6), the overall conformational entropy brought by the side chains is reduced. While this is expected to reduce the solubility of the polymer, we find the solubility to be sufficient for processing useful HTL films. Turning to the DSC, the thermal transition associated with side chain melting was eliminated, and the backbone melting temperature is pushed to temperatures above 200° C. This relatively high single thermal transition at 228° C. for TPT-T (MB/C6) is indicative of enhanced thermochemical stability at temperatures up to near 200° C. In general, smaller backbone and side chain structures of conjugated polymers exhibit fewer thermal changes due to more compact packing of polymer chains, which enhances intermolecular interactions and reduces molecular motion. (23-25)

### Optoelectronic Properties

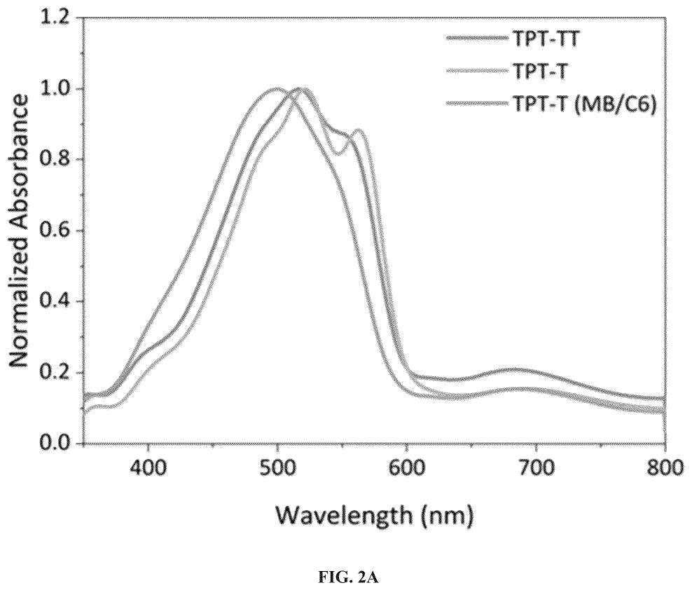

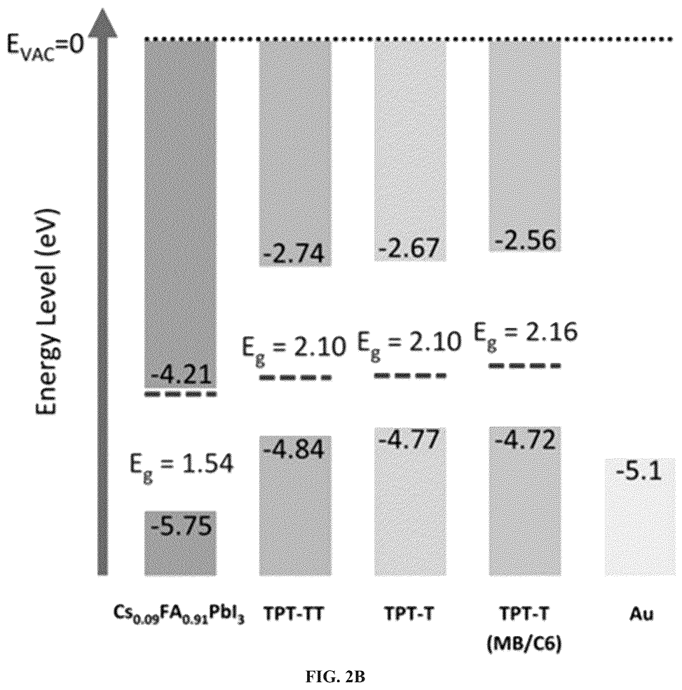

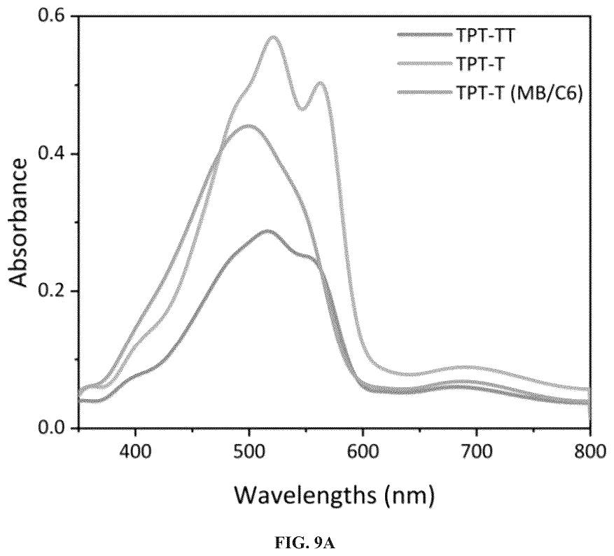

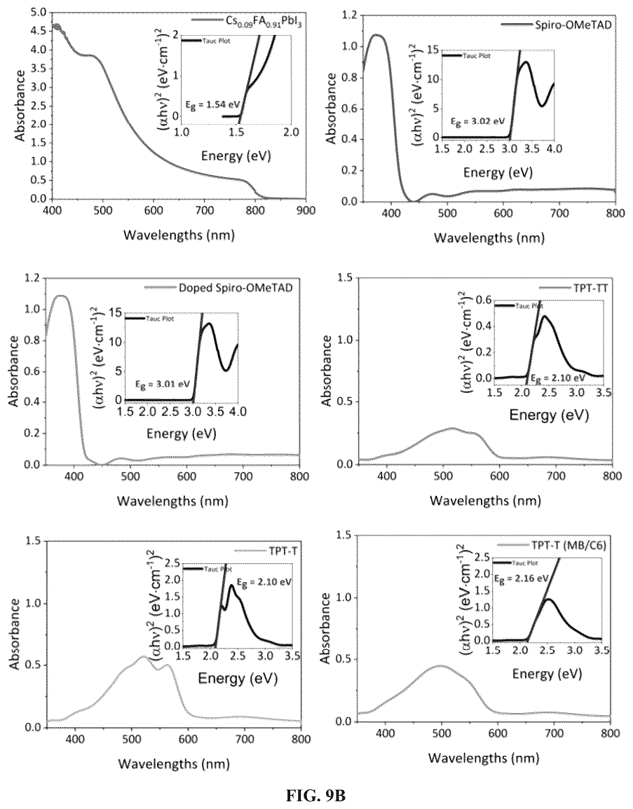

The normalized UV-vis absorption spectra of TPT-TT, TPT-T, and TPT-T (MB/C6) in thin films cast from chlorobenzene at a concentration of 20 mg mL⁻¹ via spin coating are shown in FIG. 2A. From TPT-TT to TPT-T, the absorption maximum is slightly red-shifted, with the formation of a distinct shoulder peak at 562 nm. TPT-T (MB/C6) shows a blue shift, without any noticeable shoulder peak, when compared to both TPT-T and TPT-TT. Using the onset absorption wavelength of the polymer films, the optical energy band gap (E_{g}) was also calculated via Tauc's relationship (FIGS. 9A-9B). The E_{g} for both TPT-TT and TPT-T was calculated to be around 2.10 eV, whereas that of TPT-T (MB/C6) was 2.16 eV.

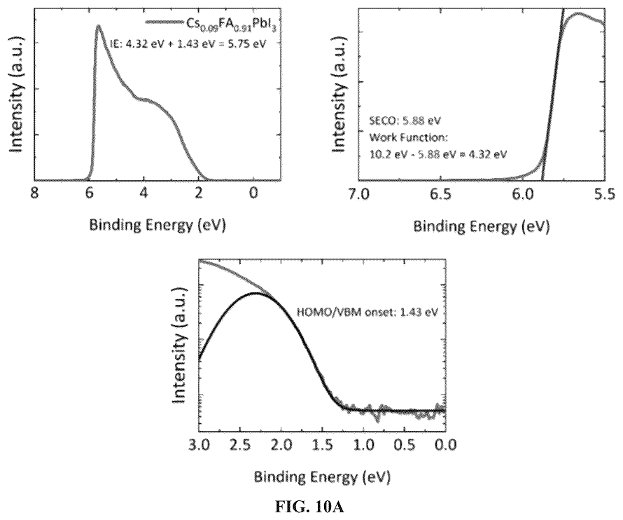

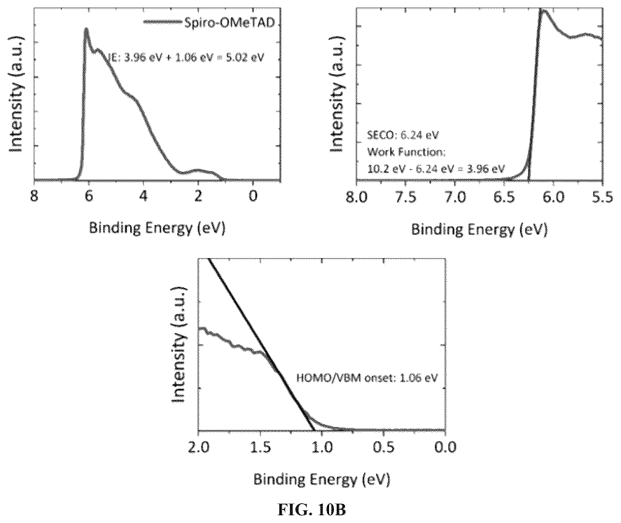

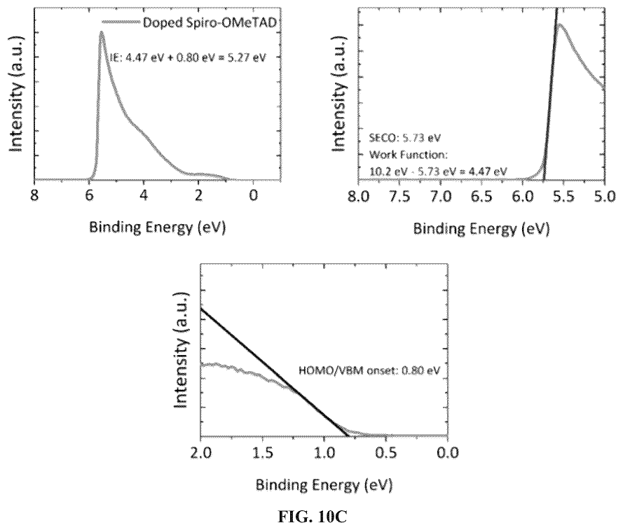

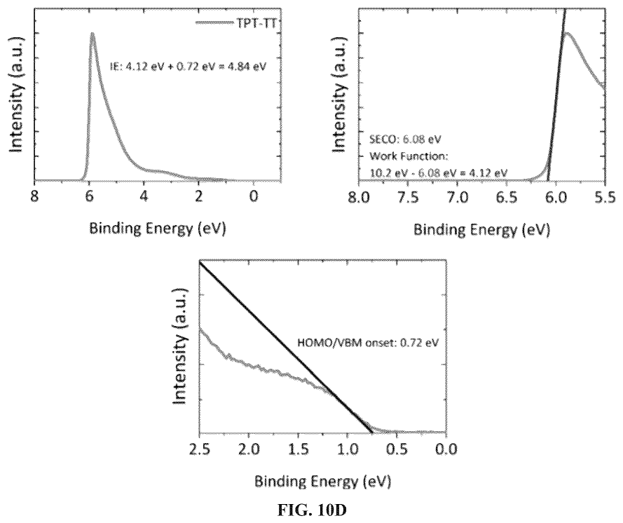

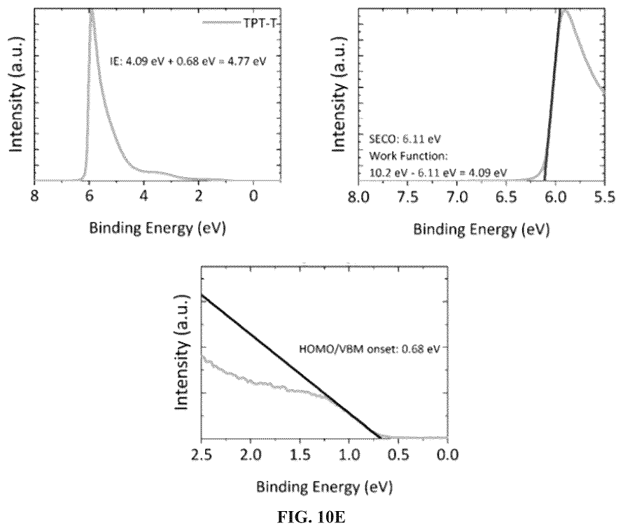

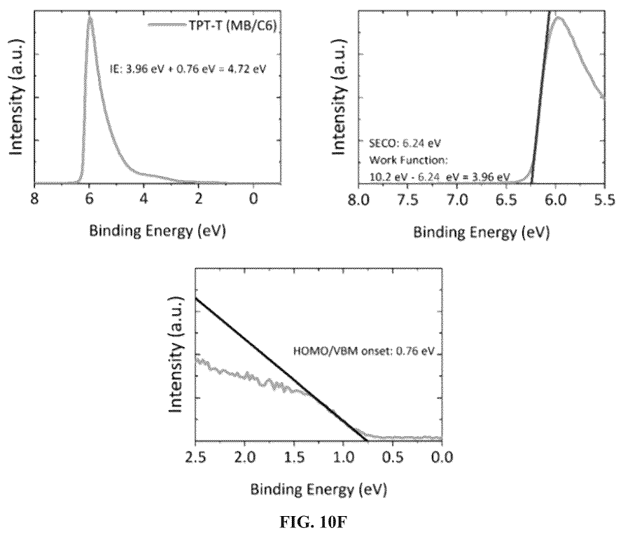

To understand the effects of backbone and side chain modifications in TPT-based polymers on their energetics, we conducted ultraviolet photoelectron spectroscopy (UPS) (FIGS. 10A-10F). The band energy alignments of Cs_{0.09}FA_{0.91}PbI₃ (CsFA) perovskite and TPT-based polymers are depicted in FIG. 2B (detailed energy positions are shown in Table 2 and FIG. 11). TPT-TT shows a HOMO energy (ionization energy) of −4.84 eV. The replacement of thienothiophene with thiophene unit (TPT-T) is accompanied by a slight increase of the HOMO energy level to −4.77 eV. The high energy difference of more than 0.5 eV between the Fermi level and the HOMO level in TPT-based polymers indicates these materials should have relatively low conductivities. Moreover, TPT-T and TPT-T (MB/C6) are anticipated to exhibit electronic properties similar to those of TPT-TT, such as an out-of-plane hole mobility of (2.43±0.01)×10⁻⁴ cm² V⁻¹ s⁻¹, as minimal changes in the Fermi and HOMO levels were observed despite the structural modifications. (22)

Device Performance of PSCs with TPT-Based Polymers

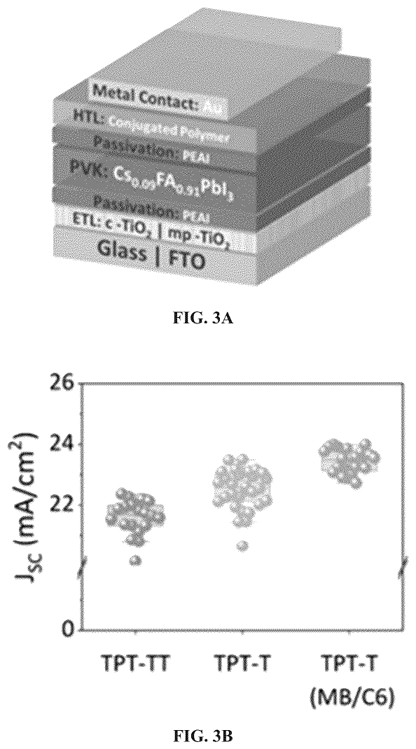

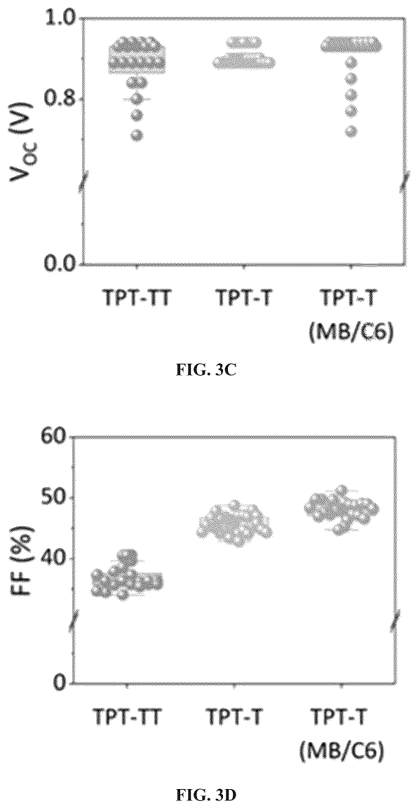

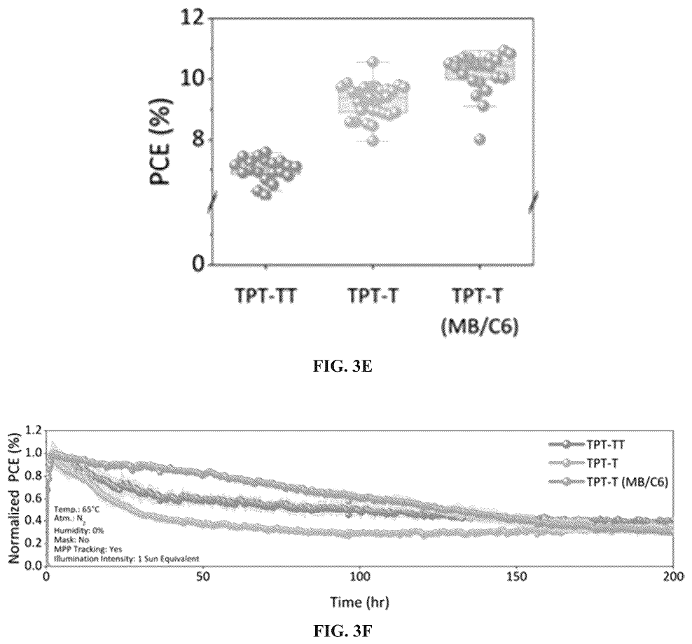

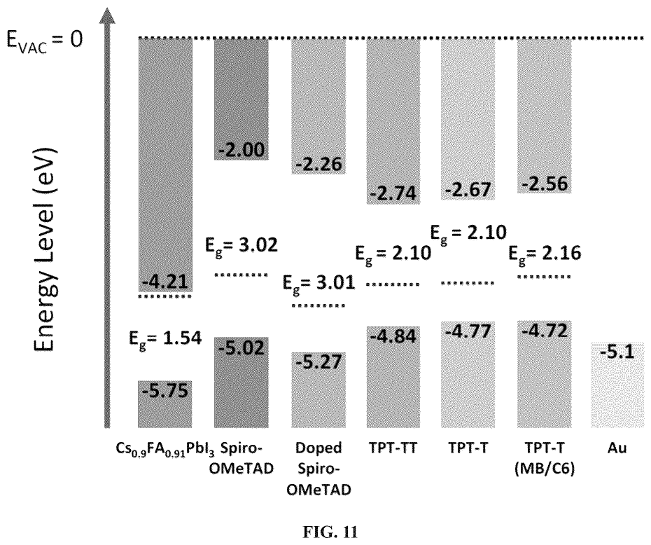

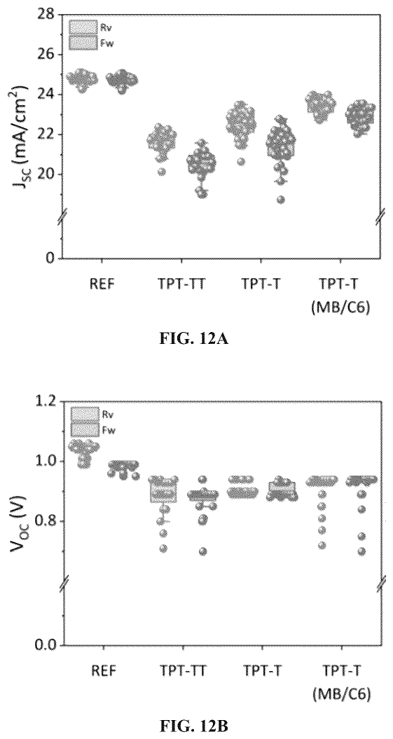

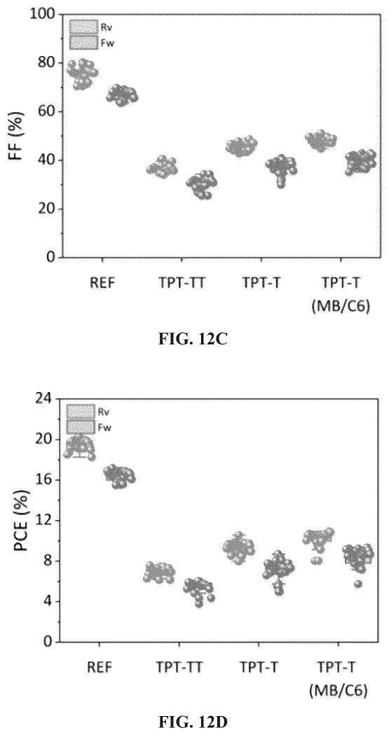

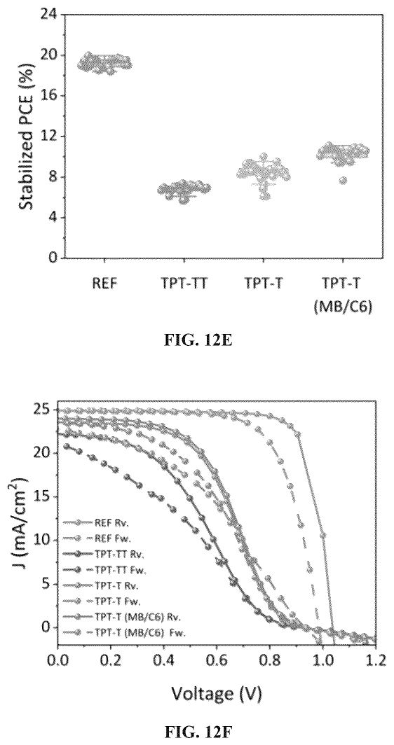

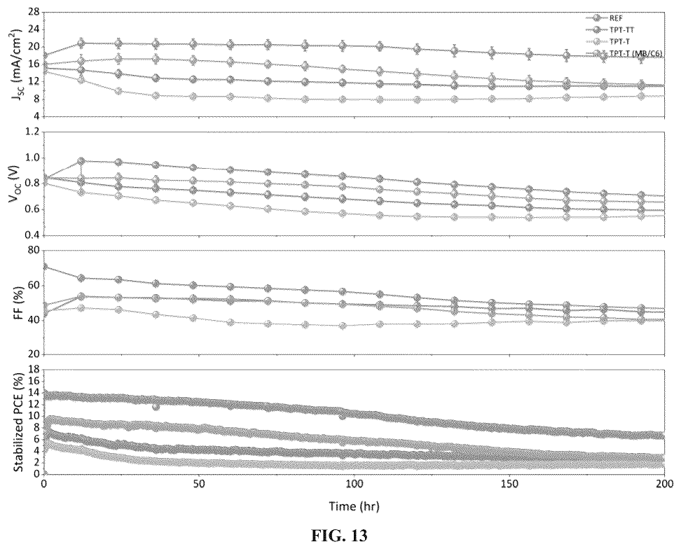

To assess the viability of TPT-based polymers as potential HTLs in PSCs, n-i-p devices consisting of fluorine-doped tin oxide/compact TiO₂/mesoporous TiO₂/phenethylammonium iodide (PEAI)/Cs_{0.09}FA_{0.91}PbI₃ (CsFA) perovskite/PEAI/CP/Au (FIG. 3A) were fabricated. The details of the PSC device fabrication are provided below. The complete device current-voltage characteristics for all devices tested are summarized FIGS. 12A-12F and Table 3. FIGS. 3B-3E display the statistical distributions of short-circuit current density (J_{sc}), open-circuit voltage (V_{oc}), fill factor (FF), and power conversion efficiency (PCE) of the devices based on different TPT-based polymer HTLs in reverse scans. Gradual increases in J_{sc} and FF were observed when modifying the polymers from TPT-TT to TPT-T (MB/C6), while V_{oc} remained relatively constant. The devices with all TPT-based polymers showed an increased series resistance when compared to those prepared using doped Spiro, as inferred from the high-voltage region in the J-V curve (FIG. 12F). S-shaped J-V curves and similar V_{oc} values of ˜0.94 eV for all TPT-based polymers suggest poor charge extraction between the polymer and Au contact, which could be due to either energy misalignment (FIG. 2B) or low hole mobilities leading to charge carrier accumulation. (26) A PCE of 11.09% (median PCE of 10.04%) was obtained in a PSC based on a TPT-T (MB/C6) HTL. Moreover, a long-term device stability study following the ISOS L-2I protocols was conducted to study the thermal effect on TPT-based polymer-incorporated PSCs, as shown in FIG. 3F. (3) The stability measurements were carried out under constant 1 sun equivalent illumination at 65° C. under a N₂ atmosphere with constant MPPT. J-V scans were performed at 12 h intervals to record the evolution in J_{sc}, V_{oc}, FF, and stabilized PCE (FIG. 13). Overall, during the 200 h of stress testing, TPT-T (MB/C6) exhibited higher performance and slower decay in stabilized PCE compared to TPT-TT and TPT-T. The improved stability of TPT-T (MB/C6) is primarily due to an unchanged V_{oc} as time progresses when compared to those of TPT-TT and TPT-T. The FF was lower than that of the TPT-TT starting at 108-h mark, while J_{sc} was similar to that of TPT-TT at the 180-h mark.

### Relation Between TPT-Based Polymer Crystallization and PSC Degradation

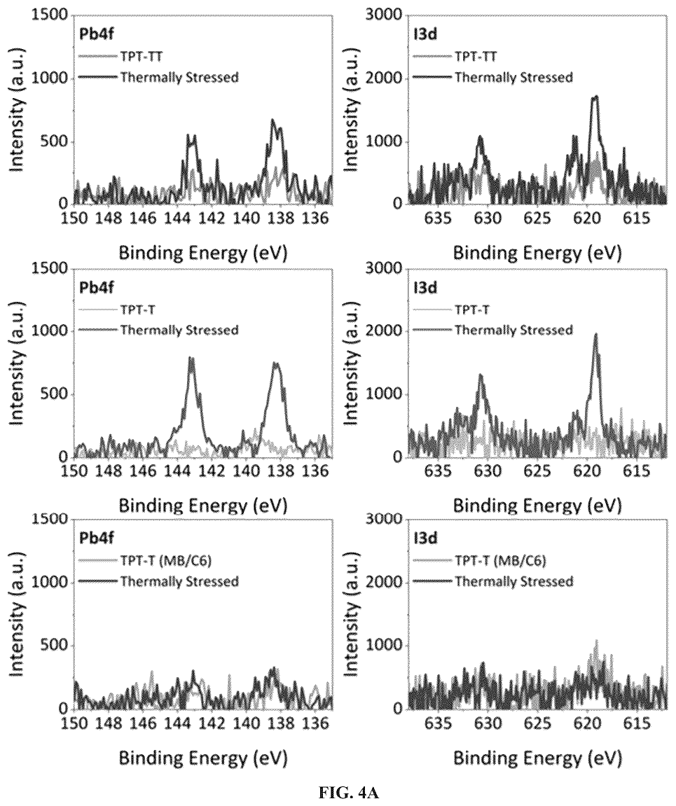

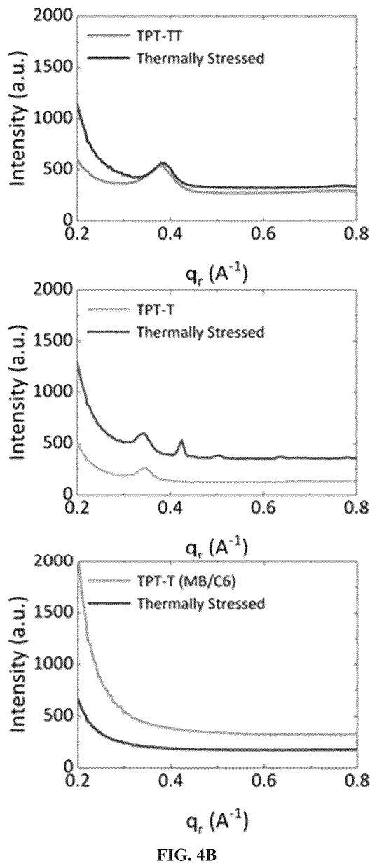

To investigate the degradation process induced by the ISOS L-2I long-term stability measurement on TPT-based polymer-incorporated PSCs, X-ray photoelectron spectroscopy (XPS) was performed on CPs on top of pristine and aged (for over 200 h at 65° C.) devices. FIG. 4A presents the XPS elemental scans of Pb 4f and I 3d of pristine and thermally stressed polymers on the completed PSCs. For all pristine polymers, no significant Pb 4f and I 3d peaks were observed. On the other hand, the Pb 4f and I 3d peaks became more prominent in TPT-TT- and TPT-T-coated films after 200 h of stability test at 65° C. This suggests that the Pb and I migrate through the polymer to be detected at the surface or that cracks have formed, and we are able to detect those elements through those openings. However, no significant changes were detected for the TPT-T (MB/C6) films, suggesting a lack of elemental migration or crack formation. We conducted scanning electron microscopy (SEM) and optical microscopy (OM) to understand whether the changes in surface chemistry detected by XPS before and after the stability test are associated with microstructural modifications. FIG. 14A shows the SEM images of pristine and thermally stressed polymers on the completed PSCs. No signs of crystallization or distinct facets were observed with SEM imaging. However, large features that resemble cracks were observed via OM on both pristine and thermally stressed Spiro, TPT-TT, and TPT-T on completed PSCs (FIG. 14B). On the other hand, no crack-like features were observed on TPT-T (MB/C6) films before or after stress testing.

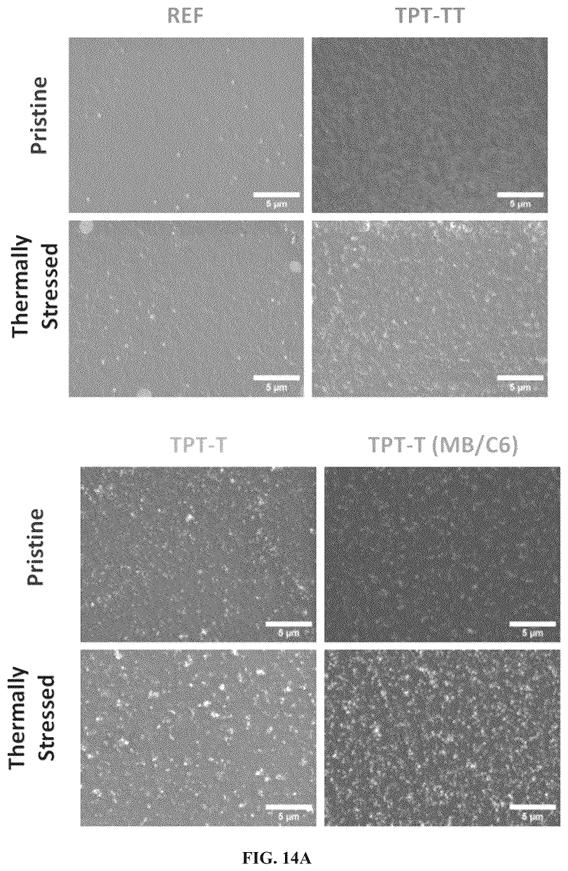

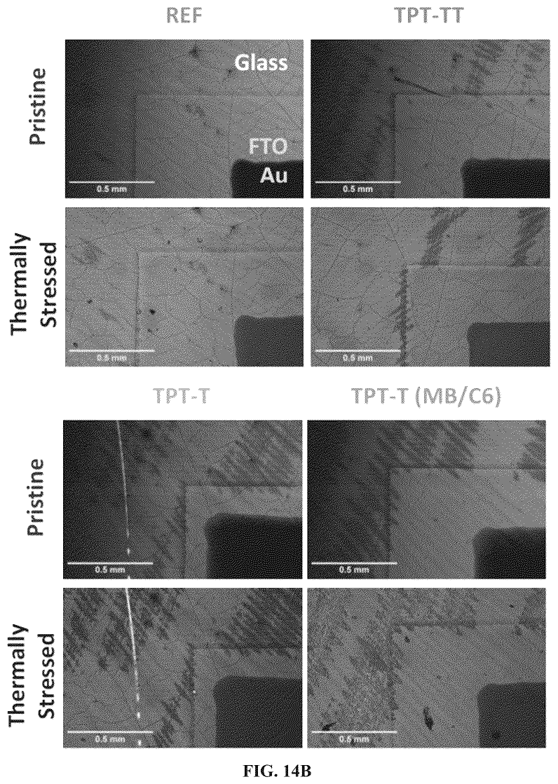

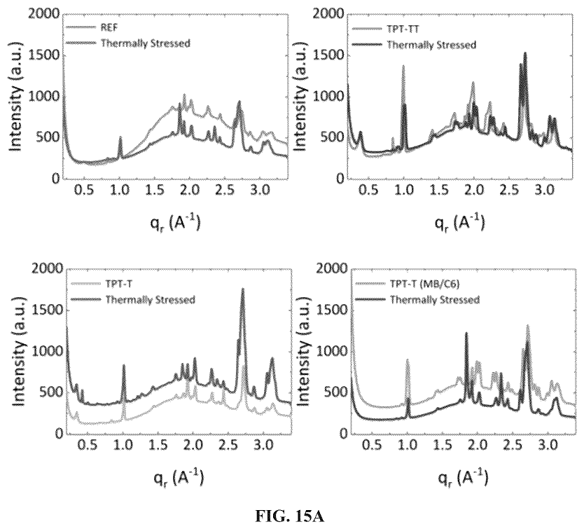

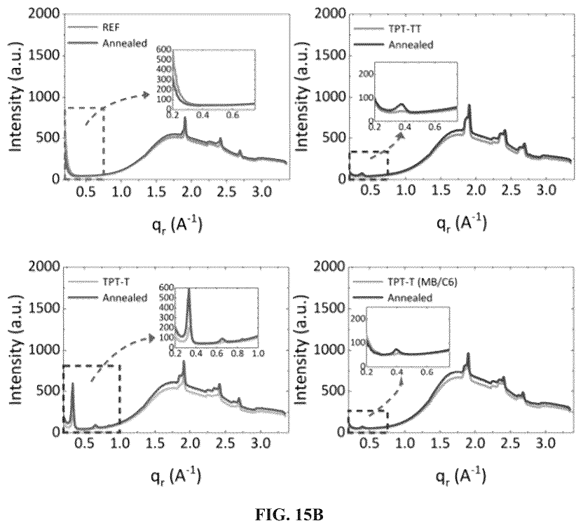

Synchrotron-based grazing incidence wide-angle X-ray scattering (GIWAXS) was performed to assess the effects of long-term stability measurements on the crystallinity of the polymers. FIG. 4B shows the 1D integrated GIWAXS profiles of pristine and thermally stressed polymers on completed PSCs. The GIWAXS profiles that include the perovskite signals are shown in FIG. 15A. To disentangle the GIWAXS signals of polymers from those of the perovskite layer, we acquired GIWAXS patterns of the polymer thin films deposited on FTO substrates from a 20 mg mL⁻¹ solution. The patterns were obtained for films before and after annealing at 100° C. for 20 min in a N₂ environment to simulate high-temperature aging (FIG. 15B). For TPT-TT and TPT-T, crystalline peaks were present for pristine films at qᵣ=0.37 A⁻¹ and qᵣ=0.34 A⁻¹, respectively. After the thermal stability test, the peaks remained relatively unchanged, while a new peak surfaced for TPT-T at qᵣ=0.42 A⁻¹. Importantly, no crystalline peaks were present for the TPT-T (MB/C6)-incorporated PSC before and after the thermal stability test. Since the TPT-T (MB/C6) thin film on FTO substrate showed a small crystalline peak at 0.41 A⁻¹, the result suggests that the layer under the polymer also plays an important role in its crystallization. (2)

We previously discussed that the introduction of short branched side chains leads to high thermal transition temperatures in TPT-T (MB/C6) (FIGS. 1A-1B). Our DSC, XPS, OM, GIWAXS, and device stability measurements show a correlation between the temperature at which thermal transitions occur and crystallization in polymer thin films. We believe that the higher temperatures for thermal transitions in TPT-T (MB/C6) are needed to produce a more amorphous as-cast film that does not crystallize at typical operation temperatures of solar cells. The lack of lower-temperature thermal transitions (below 200° C.) is needed for more stable solar cells. On the other hand, the DSC data shows thermal transitions at lower temperatures (below 200° C.) for both TPT-TT and TPT-T, which coincide with crystalline peak formation and cracking for both as-cast and thermally stressed thin films. This, in turn, leads to solar cells that rapidly lose efficiency during thermal stress.

### Effect of LiTFSI and tBP Additives on TPT-T (MB-C6)

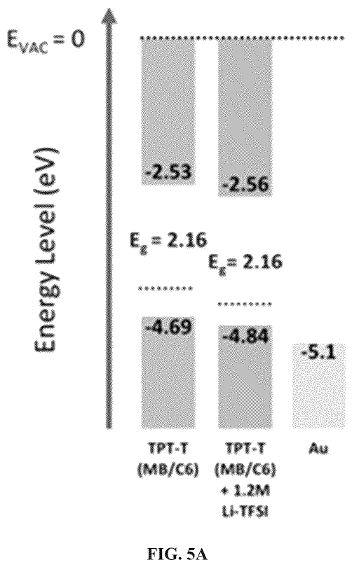

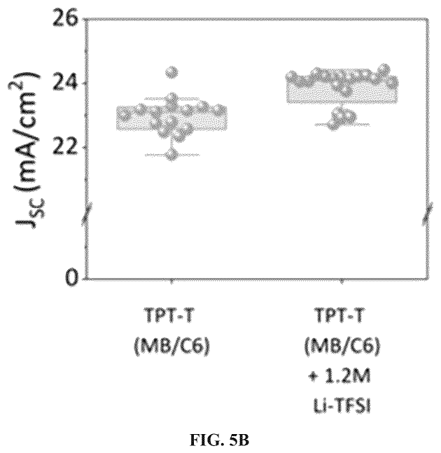

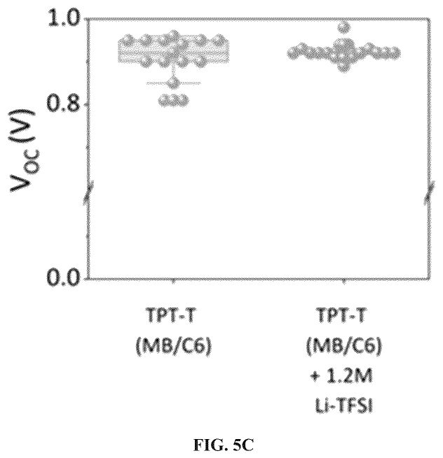

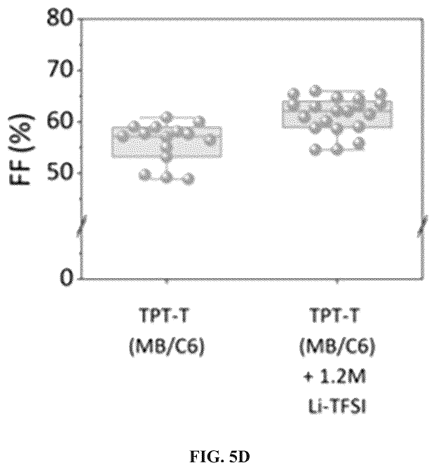

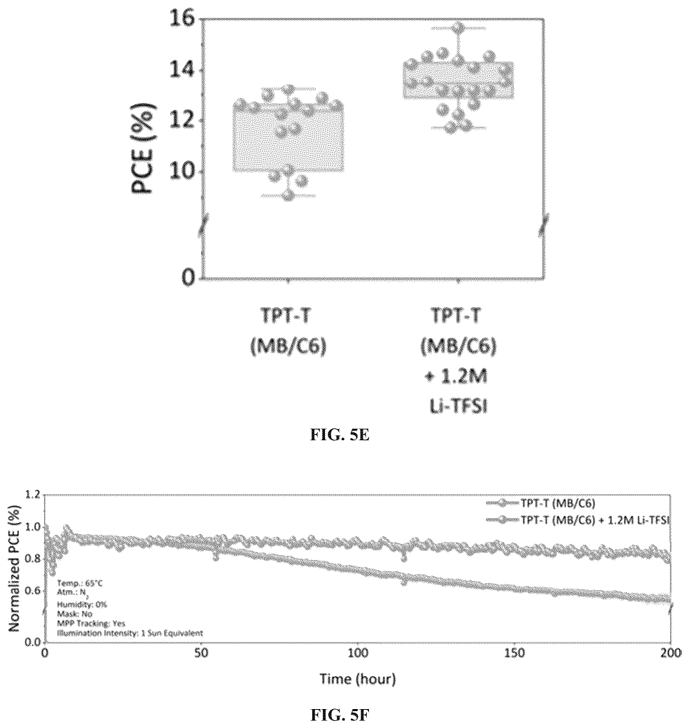

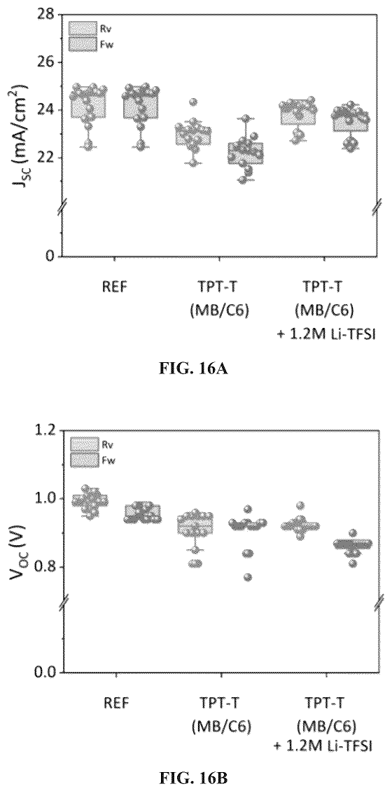

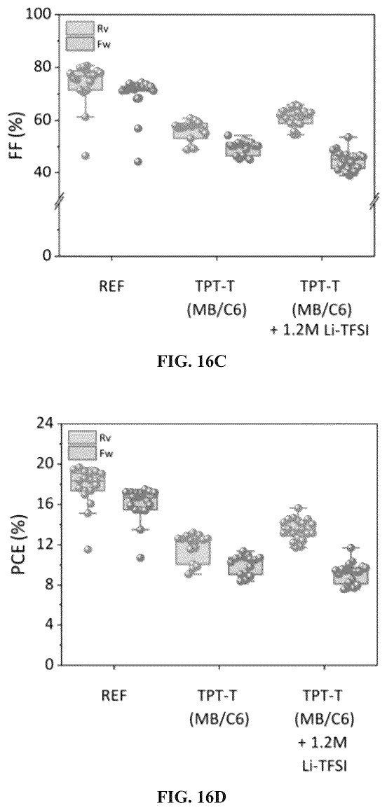

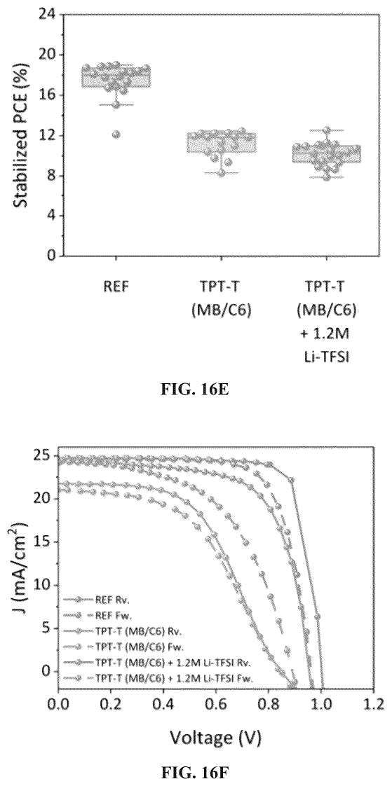

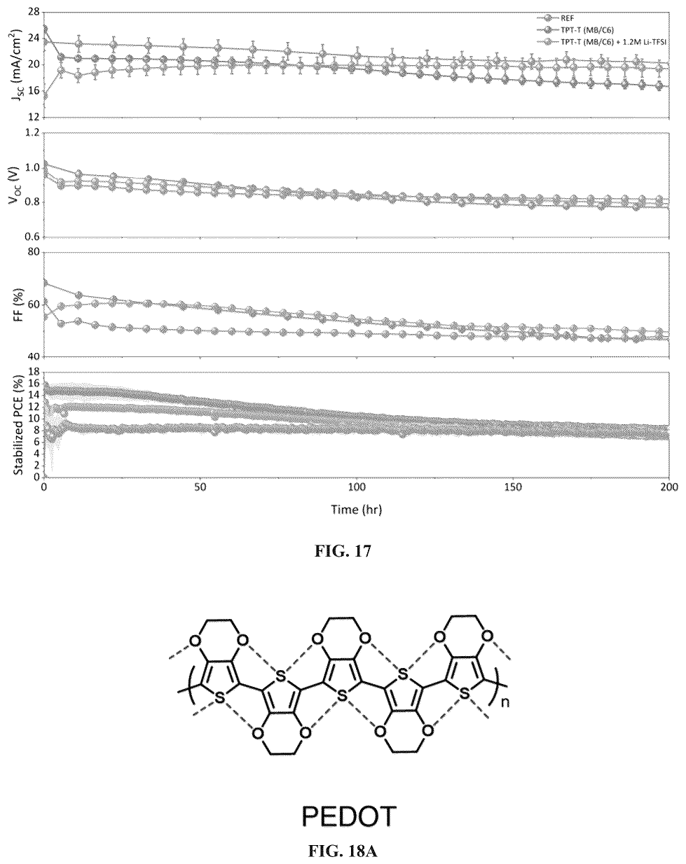

Having identified TPT-T (MB/C6) as a candidate material for high efficiency and improved long-term stability, we added Li-TFSI with tBP to improve its electronic properties, as is commonly done for such polymeric HTLs. The energy band positions of TPT-T (MB/C6), both with and without the 1.2 M Li-TFSI and tBP additives, are illustrated in FIG. 5A (detailed energy positions are shown in FIGS. 16A-16F). The addition of these dopants resulted in a reduction of both the Fermi level and the HOMO energy level, compared to those of the undoped TPT-T (MB/C6). FIGS. 5B-5E the statistical distributions of J_{sc}, V_{oc}, FF, and PCE of TPT-T (MB/C6) and doped TPT-T (MB/C6) with 1.2 M Li-TFSI and tBP additives; the detailed photovoltaic parameters are reported in Table 4. Overall improvements in J_{sc}, V_{oc}, and FF are observed in the doped TPT-T (MB/C6). The S-shaped J-V curve that was originally measured for TPT-T (MB/C6) disappeared for the additive-based devices (FIG. 16F), which is attributed to better matching of the HOMO energy level of the HTL to the work function of the Au contact. Furthermore, the lower Fermi level in doped TPT-T (MB/C6) suggests a higher concentration of positive charge carriers, enhancing the conductivity. These improvements led to PCE reaching a maximum of 15.65% for the doped TPT-T (MB/C6). Following the ISOS L-2I protocols, the additive-free and the TPT-T (MB/C6) polymer with additives were subjected to a long-term stability test under 1 sun equivalent illumination at 65° C. in a N₂ atmosphere with constant MPPT (FIG. 5F). A J-V scan was conducted every 12 h to track the changes in J_{sc}, V_{oc}, FF, and stabilized PCE (FIG. 17). Interestingly, the doped TPT-T (MB/C6) did not show much of a change in the PCE for up to 200 h. It is possible that the improved stability of the TPT-T (MB/C6) with additives is due to improved energy level alignment, which is directly related to ion movement in perovskite solar cells. (27)

### CONCLUSION

A series of thiophene-based CP HTLs have been successfully synthesized, characterized, and incorporated into n-i-p PSCs. Among the three CP variants examined, TPT-T (MB/C6) with shorter, branched side chains exhibited superior device performance and exceptional long-term stability. This achievement can be attributed to its amorphous nature, which helps prevent cracking during thermal stress testing. Notably, the TPT-TT and TPT-T polymers were found to undergo faster degradation, marked by the development of crystalline domains and macroscale cracks after thermal stress testing in solar cells. These cracks exposed the underlying perovskite layer. Furthermore, the combination of Li-TFSI and tBP additives with TPT-T (MB/C6) yielded PCE above 15%, which can be attributed to better band alignment and improved electronic properties as the work function increases. Remarkably, TPT-T (MB/C6) with additives exhibited exceptional thermal stability for over 200 h at 65° C. This example introduces a novel chemical structure design for thiophene-based CPs that not only exhibit thermal resistance but also are compatible with dopants, offering an avenue to enhance the long-term stability of PSCs. Furthermore, this example shows an important correlation between thermal transition temperatures and CP thin film crystallization that occurs within the temperatures at which solar cells are tested.

### REFERENCES FOR EXAMPLE 1

1. Best Research-Cell Efficiency Chart|Photovoltaic Research|NREL. https.//www.nrel.gov/pv/cell-efficiency.html (accessed 2023 Sep. 18).
2. Shao, S.; Loi, M. A. The Role of the Interfaces in Perovskite Solar Cells. Adv. Mater. Interfaces 2020, 7 (1), 1901469.
3. Khenkin, M. V.; Katz, E. A.; Abate, A.; Bardizza, G.; Berry, J. J.; Brabec, C.; Brunetti, F.; Bulović, V.; Burlingame, Q.; Di Carlo, A.; Cheacharoen, R.; Cheng, Y. B.; Colsmann, A.; Cros, S.; Domanski, K.; Dusza, M.; Fell, C. J.; Forrest, S. R.; Galagan, Y; Di Girolamo, D.; Grätzel, M.; Hagfeldt, A.; von Hauff, E.; Hoppe, H.; Kettle, J.; Köbler, H.; Leite, M. S.; Liu, S.; Loo, Y. L.; Luther, J. M.; Ma, C. Q.; Madsen, M.; Manceau, M.; Matheron, M.; McGehee, M.; Meitzner, R.; Nazeeruddin, M. K.; Nogueira, A. F.; Odabaşi, Ç.; Osherov, A.; Park, N. G.; Reese, M. O.; De Rossi, F.; Saliba, M.; Schubert, U. S.; Snaith, H. J.; Stranks, S. D.; Tress, W.; Troshin, P. A.; Turkovic, V; Veenstra, S.; Visoly-Fisher, I.; Walsh, A.; Watson, T.; Xie, H.; Yildinm, R.; Zakeeruddin, S. M.; Zhu, K.; Lira-Cantu, M. Consensus Statement for Stability Assessment and Reporting for Perovskite Photovoltaics Based on ISOS Procedures. Nature Energy 2020, 5 (1), 35-49.
4. Zhu, H.; Teale, S.; Lintangpradipto, M. N.; Mahesh, S.; Chen, B.; McGehee, M. D.; Sargent, E. H.; Bakr, O. M. Long-Term Operating Stability in Perovskite Photovoltaics. Nat. Rev. Mater. 2023, 8 (9), 569-586.
5. Wang, S.; Huang, Z.; Wang, X.; Li, Y; Günther, M.; Valenzuela, S.; Parikh, P.; Cabreros, A.; Xiong, W.; Meng, Y S. Unveiling the Role of TBP-LiTFSI Complexes in Perovskite Solar Cells. J. Am. Chem. Soc. 2018, 140 (48), 16720-16730.
6. Li, Z.; Xiao, C.; Yang, Y; Harvey, S. P.; Kim, D. H.; Christians, J. A.; Yang, M.; Schulz, P.; Nanayakkara, S. U.; Jiang, C. S.; Luther, J. M.; Berry, J. J.; Beard, M. C.; Al-Jassim, M. M.; Zhu, K. Extrinsic Ion Migration in Perovskite Solar Cells. Energy Environ. Sci. 2017, 10 (5), 1234-1242.
7. Zhao, X.; Kim, H.-S.; Seo, J.-Y; Park, N.-G. Effect of Selective Contacts on the Thermal Stability of Perovskite Solar Cells. ACS Appl. Mater. Interfaces 2017, 9 (8), 7148-7153.
8. Malinauskas, T.; Tomkute-Luksiene, D.; Sens, R.; Daskeviciene, M.; Send, R.; Wonneberger, H.; Jankauskas, V.; Bruder, I.; Getautis, V. Enhancing Thermal Stability and Lifetime of Solid-State Dye-Sensitized Solar Cells via Molecular Engineering of the Hole-Transporting Material Spiro-OMeTAD. ACS Appl. Mater Interfaces 2015, 7 (21), 11107-11116.
9. Song, W.; Rakocevic, L.; Thiruvallur Eachambadi, R.; Qiu, W.; Bastos, J. P.; Gehlhaar, R.; Kuang, Y; Hadipour, A.; Aernouts, T.; Poortmans, J. Improving the Morphology Stability of Spiro-OMeTAD Films for Enhanced Thermal Stability of Perovskite Solar Cells. ACS Appl. Mater Interfaces 2021, 13 (37), 44294-44301.
10. Domanski, K.; Correa-Baena, J.-P.; Mine, N.; Nazeeruddin, M. K.; Abate, A.; Saliba, M.; Tress, W.; Hagfeldt, A.; Grätzel, M. Not All That Glitters Is Gold: Metal-Migration-Induced Degradation in Perovskite Solar Cells. ACS Nano 2016, 10 (6), 6306-6314.
11. Jeong, S.-Y; Kim, H.-S.; Park, N.-G. Challenges for Thermally Stable Spiro-MeOTAD toward the Market Entry of Highly Efficient Perovskite Solar Cells. ACS Appl. Mater Interfaces 2022, 14 (30), 34220-34227.
12. Reynolds, J. R.; Thompson, B. C.; Skotheim, T. A. Handbook of Conducting Polymers, 4th ed.; CRC Press: Boca Raton, 2019.
13. Hawash, Z.; Ono, L. K.; Qi, Y Recent Advances in Spiro-MeOTAD Hole Transport Material and Its Applications in Organic-Inorganic Halide Perovskite Solar Cells. Adv. Mater. Interfaces 2018, 5 (1), 1700623.
14. Rombach, F. M.; Haque, S. A.; Macdonald, T. J. Lessons Learned from Spiro-OMeTAD and PTAA in Perovskite Solar Cells. Energy Environ. Sci. 2021, 14 (10), 5161-5190.
15. Saliba, M.; Matsui, T.; Domanski, K.; Seo, J.-Y; Ummadisingu, A.; Zakeeruddin, S. M.; Correa-Baena, J.-P.; Tress, W. R.; Abate, A.; Hagfeldt, A.; Gratzel, M. Incorporation of Rubidium Cations into Perovskite Solar Cells Improves Photovoltaic Performance. Science (1979) 2016, 354 (6309), 206-209.
16. Intaniwet, A.; Mills, C. A.; Shkunov, M.; Thiem, H.; Keddie, J. L.; Sellin, P. J. Characterization of Thick Film Poly(Triarylamine) Semiconductor Diodes for Direct x-Ray Detection. J. Appl. Phys. 2009, 106 (6), 64513.
17. Barard, S.; Heeney, M.; Chen, L.; Colle, M.; Shkunov, M.; Mcculloch, I.; Stingelin, N.; Philips, M.; Kreouzis, T. Separate Charge Transport Pathways Determined by the Time of Flight Method in Bimodal Polytriarylamine. J. Appl. Phys. 2009, 105 (1), 13701.
18. Liao, Q.; Wang, Y; Yao, X.; Su, M.; Li, B.; Sun, H.; Huang, J.; Guo, X. A Dual-Functional Conjugated Polymer as an Efficient Hole-Transporting Layer for High-Performance Inverted Perovskite Solar Cells. ACS Appl. Mater. Interfaces 2021, 13 (14), 16744-16753.
19. Chang, C. Y; Huang, H. H.; Tsai, H.; Lin, S. L.; Liu, P. H.; Chen, W; Hsu, F. C.; Nie, W.; Chen, Y. F.; Wang, L. Facile Fabrication of Self-Assembly Functionalized Polythiophene Hole Transporting Layer for High Performance Perovskite Solar Cells. Advanced Science 2021, 8 (5), 2002718.
20. Cinar, M. E.; Ozturk, T. Thienothiophenes, Dithienothiophenes, and Thienoacenes: Syntheses, Oligomers, Polymers, and Properties. Chem. Rev. 2015, 115 (9), 3036-3140.
21. Barbarella, G.; Melucci, M.; Sotgiu, G. The Versatile Thiophene: An Overview of Recent Research on Thiophene-Based Materials. Adv. Mater. 2005, 17 (13), 1581-1593.
22. Sabury, S.; Xu, Z.; Saiev, S.; Davies, D.; Osterholm, A. M.; Rinehart, J. M.; Mirhosseini, M.; Tong, B.; Kim, S.; Correa-Baena, J. P.; Coropceanu, V.; Jurchescu, O. D.; Brédas, J. L.; Diao, Y; Reynolds, J. R. Non-Covalent Planarizing Interactions Yield Highly Ordered and Thermotropic Liquid Crystalline Conjugated Polymers. Mater. Horiz 2024, 11 (14), 3352-3363.
23. Pankaj, S.; Hempel, E.; Beiner, M. Side-Chain Dynamics and Crystallization in a Series of Regiorandom Poly(3-Alkylthiophenes). Macromolecules 2009, 42 (3), 716-724.
24. Chang, M.; Meng, L.; Wang, Y; Ke, X.; Yi, Y-Q.-Q.; Zheng, N.; Zheng, W.; Xie, Z.; Zhang, M.; Yi, Y; Zhang, H.; Wan, X.; Li, C.; Chen, Y Achieving an Efficient and Stable Morphology in Organic Solar Cells Via Fine-Tuning the Side Chains of Small-Molecule Acceptors. Chem. Mater. 2020, 32 (6), 2593-2604.
25. Müller, C. On the Glass Transition of Polymer Semiconductors and Its Impact on Polymer Solar Cell Stability. Chem. Mater. 2015, 27 (8), 2740-2754.
26. Saive, R. S-Shaped Current-Voltage Characteristics in Solar Cells: A Review. IEEE J. Photovolt 2019, 9 (6), 1477-1484.
27. Kerner, R. A.; Heo, S.; Roh, K.; MacMillan, K.; Larson, B. W.; Rand, B. P. Organic Hole Transport Material Ionization Potential Dictates Diffusion Kinetics of Iodine Species in Halide Perovskite Devices. ACS Energy Lett. 2021, 6 (2), 501-508.

### Supplementary Experimental Details for Example 1

### Experimental Methods

TPT-based Conjugated Polymer Materials & Synthesis: The general synthesis scheme for the TPT-based conjugated polymers including the synthesis of the intermediate molecules is shown in FIG. 6. Side chains are generalized in this figure, labeled as R₁ (side chain attached to the phenyl ring) and R₂ (side chain attached to the flanking thiophene rings). R₁ and R₂ are described for each polymer in FIG. 6. Details of each synthesis step and the polymerization conditions are reported herein.¹ The nature of the alkyl side chains (length and linear vs branched) did not affect the synthesis procedure.

Perovskite Solar Cell Fabrication: Patterned fluorine-doped tin oxide (FTO) glass substrates (7 Ω·sq⁻¹) were consecutively cleaned using 2% Mucasol (schülke) solution, deionized water, acetone (Sigma-Aldrich, ≥99.5%), and isopropyl alcohol (IPA, Fisher Chemical) via 15 min of ultrasonication. The cleaned substrates were then dried with N₂ gun and put under UV-ozone treatment for 15 min. For an electron transport layer, a compact TiO₂ (c-TiO₂) layer was deposited by spray pyrolysis from a solution containing 480 μL acetylacetone (Sigma-Aldrich, ≥99%), 720 μL titanium diisopropoxide bis(acetylacetonate) 75 wt. % in isopropanol (Sigma-Aldrich), and 10.8 mL of ethanol (Sigma-Aldrich, anhydrous, ≥99.5%). The prepared solution was sprayed onto the preheated substrates at 450° C. with 30 s interval between each cycle and then post-annealed for 30 min. A single cycle includes 16 s to 18 s of spraying time, and 3 L min-1 flow of O₂ was utilized as a carrier gas. After the substrates were cooled down to room temperature, 60 μL of mesoporous-TiO₂ (mp-TiO₂) solution was static spin coated at a speed of 4000 rpm for 10 s with an acceleration rate of 4000 rpm s⁻¹; mp-TiO₂ solution consists of 150 mg mL⁻¹ diluted TiO₂ paste (Sigma-Aldrich) in anhydrous ethanol (Sigma-Aldrich, ≥99.5%). As-prepared Glass|FTO|c-TiO₂|mp-TiO₂ stack substrates were desiccated using a hotplate at 100° C. for 10 min and subsequently sintered at 450° C. for 30 min. From cleaning of the substrates to spin coating mp-TiO₂ layer was carried out in ambient air and then transferred to N₂ filled glove box.

A 90 μL of phenethyl ammonium iodide (PEAI, Dynamo) solution with a concentration of 1 mg mL⁻¹ in IPA (Sigma-Aldrich, 99.9%), was spin coated on top of mp-TiO₂ for 20 s at 5000 rpm with accelerate rate of 5000 rpm s⁻¹. Subsequently, 1.2M Cs_{0.09}FA_{0.91}PbI₃ (CsFA) perovskite thin film with 5% excess Pb was deposited using a two-step spin coating process. CsFA solution was prepared by co-dissolving cesium iodide (Sigma-Aldrich), formamidinium iodide (Dynamo), and lead iodide (Tokyo Chemical Industry, >98.0%) in 2:1 volume ratio of N,N-dimethyl formamide (DMF, Acros Organics, ≥99.8%):dimethyl sulfoxide (Acros Organics, 99.8+%). A 90 μL of CsFA perovskite solution was spun at 1000 rpm with acceleration rate of 1000 rpm for 10 s followed by 6000 rpm s⁻¹ for 20 s with acceleration rate of 6000 rpm s⁻¹. CsFA perovskite solution was made sure to spread completely over the substrate with a pipette tip before spin coating. Afterwards, 250 μL of chlorobenzene (Sigma-Aldrich, anhydrous, 99.8%) was dynamically spin coated 3 s before the end of the second step, and then annealed at 150° C. for 10 min. The same PEAI surface treatment mentioned above was incorporated on top of the CsFA film and then annealed at 100° C. for 10 min. For the hole transport layer (HTL), a doped Spiro-OMeTAD and 20 mg mL⁻¹ of TPT-TT, TPT-T, and TPT-T (MB/C6) in chlorobenzene solution were dynamically spin-cast on the PEAI treated CsFA perovskite film. All of the HTL solutions were prepared at room temperature except for TPT-TT polymer solution which was separately pre-heated at 45° C. for 5 min for better dissolution.

A doped Spiro-OMeTAD consists of 100 mg of Spiro-OMeTAD (1 Material) dissolved in 1098.38 μL of chlorobenzene (Acros Organics, 99.9%) to form a 0.07M solution, 18.13 μL of 1.8M lithium bis(trifluoromethane)sulfonimide (Li-TFSI, Sigma-Aldrich) in acetonitrile (Sigma-Aldrich, anhydrous, 99.8%), 39.45 μL of 4-tertbutylpyridine (tBP, Sigma-Aldrich, 98%) and 9.79 μL of 0.25M tris(2-(1H-pyrazol-1-yl)-4-tert-butylpyridine)cobalt(III) tri[bis(trifluoromethane)sulfonimide](FK 209 Co (III), Sigma-Aldrich) in acetonitrile; 90 μL of doped Spiro-OMeTAD solution is spin coated dynamically with 3000 rpm for 30 s with an acceleration of 3000 rpm s⁻¹. For TPT-based polymers, 65 μL of polymer solution was dynamically spin coated at a speed of 4000 rpm for 20 seconds with acceleration of 4000 rpm s⁻¹. The solution processing from PEAI treatment to spin coating of hole transport layer was carried out in N₂ filled glove box with O₂ and H₂O content below 4 ppm with temperature ranging from 18° C. to 24° C. The edges of the substrates were cleaned to remove CsFA and hole transport layer with DMF and followed by acetonitrile in ambient air. Finally, 50 nm of Au (Kurt J. Lesker, 99.999%) were thermally evaporated as the back contact using a shadow mask to make 8 independent cells in a single substrate. The active device area is 0.128 cm².

### Doping of TPT-T (MB/C6):

Doped TPT-T (MB/C6) was prepared by mixing 20 mg mL⁻¹ of TPT-T (MB/C6) with 0.084 mol-to-mol ratio of TPT-T (MB/C6) solution to Li-TFSI (1.2M in acetonitrile) and 0.39 mol-to-mol ratio of TPT-T (MB/C6) to tBP.

### Characterization

Elemental Analysis (EA): EA was conducted via Atlantic Microlab Inc. to calculate polymer repeat units.

Gel Permeation Chromatography (GPC): The number average molecular weight (Mn), weight average molecular weight (Mw), and dispersity (Ð) were determined using a Tosoh EcoSEC high temperature GPC instrument with RI detector.

H¹ Nuclear Magnetic Resonance (NMR): ¹H NMR spectra for all monomers and molecular precursors were acquired through Bruker Avance IIIHD 500 MHz or Bruker Avance IIIHD 700 MHz instruments using CDCl₃ as solvent; the residual CHCl₃ peak was used as a reference for all reported chemical shifts (1H: δ=7.26 ppm, 13C: δ=77.16 ppm).

Differential Scanning Calorimetry (DSC): DSC was performed using a TA Instruments Q200 with heating and cooling rates of 10° C. min⁻¹. Powder samples with a mass about were used and encapsulated in closed DSC aluminum pan under a controlled N₂ atmosphere.

Ultraviolet-visible (UV-Vis) Spectroscopy: Absorption spectra were measured using Cary 5000 UV-Vis NIR spectrophotometer. The film was prepared by spin-coating 20 mg mL⁻¹ polymer solution dissolved in chlorobenzene at room temperature on bare FTO substrate; TPT-TT polymer solution was separately pre-heated at 45° C. for 5 min for better dissolution. The spin coating recipe is dynamic with 65 μL polymer solution at a speed of 4000 rpm for 20 seconds with acceleration of 4000 rpm s⁻¹.

Ultraviolet Photoelectron Spectroscopy (UPS): UPS was measured inside PHI 5600 UHV chamber coupled with a hemispherical electron energy analyzer. An Excitech H Lyman-α photon source with 10.2 eV photon energy was used. The path of the light was filled with nitrogen. A sample bias of −5 V and a pass energy of 5.85 eV was used during the measurement. The secondary electron cut off is determined by linear fitting in both polymer and perovskite samples and is used to calculate the work function by subtracting it from the photon source energy (10.2 eV). The highest molecular orbital (HOMO) onset for the hole transporting layers (HTL) is determined by a linear fitting to the HOMO onset, with the onset energy difference from the Fermi energy added to the work function to obtain the HOMO energy with respect to the vacuum. For the perovskite film, the valence band maximum (VBM) onset is determined by a Gaussian fitting method, as previously reported.² The combination of the optical energy band gap from UV-Vis and HOMO/VBM energy extracted from UPS is used to estimate the lowest unoccupied molecular orbital energy level (LUMO)/conduction band minimum (CBM).

All films were spin-coated on top of 0.4M CsFA perovskite thin film with 5% excess Pb on bare tin-doped indium oxide (ITO) substrate and characterized by UPS. A 90 μL of 0.4M CsFA perovskite solution was spun at 1000 rpm with acceleration rate of 1000 rpm for 10 s followed by 6000 rpm s⁻¹ for 20 s with acceleration rate of 7000 rpm s⁻¹. Subsequently, 250 μL of chlorobenzene was dynamically spin coated 3 s before the end of the second step, and then annealed at 150° C. for 10 min. For both undoped and doped Spiro-OMeTAD, 0.02M solutions were prepared. A 90 μL of solution is spin coated dynamically with 5000 rpm for 30 s with an acceleration of 5000 rpm s⁻¹. For TPT-based polymers, a 65 μL of 15 mg mL⁻¹ polymer solutions were dynamically spin coated at a speed of 6000 rpm for 20 seconds with acceleration of 6000 rpm s⁻¹.

Scanning Electron Microscopy (SEM) & Optical Microscopy (OM): SEM measurements were carried out with Hitachi SU8230 using a secondary-electron detector at 1.5 keV and 10 mA emission current. Optical microscope images were acquired with LEICA DM 2500 optical microscope.

Grazing Incidence Wide-Angle X-ray Scattering (GIWAXS): Synchrotron GIWAXS measurements were performed at beamline 11-BM at National Synchrotron Light Source II in Brookhaven National Laboratory. The X-ray beam energy of 13.5 keV with a spot size of 0.2 mm×0.05 mm. The samples were irradiated for 10 s with an incident angle of 0.05°, 0.1°, and 0.5°. Beam divergence was 1 mrad with energy resolution of 0.7%. The data were analyzed using the SciAnalysis packaged provided by the beamline.

X-ray Photoelectron Spectroscopy (XPS): XPS measurements were conducted with a Thermo Scientific K-Alpha using a monochromatic Al Kα X-ray source (hv=1486.6 eV) with a 60° incident angle and a 0° photoemission angle, both measured from the samples normal vector. Survey and high-resolution scans were measured when the chamber pressure was less than 1×10⁻⁷ torr. Survey scans were acquired averaging two measurements with 200 eV pass energy, 50 ms dwell time, and 0.1 eV step size. High-resolution scans were collected averaging 10 measurements with 50 eV pass energy, 50 ms dwell time, and 0.1 eV step size for Au4f, C1s, Cs3d, Co2p, F1s, I3d, N1s, O1s, Pb4f, S2p. Peak fitting was calculated via Thermo Scientific Advantage Data System. For all peaks, C—C peak position was used as a reference binding energies to compensate for possible surface charge shifting.

Device Characterization: Photovoltaic performance was evaluated with Fluxim Litos Lite setup equipped with a Wavelabs Sinus-70 AAA solar similar with an illumination of AM 1.5G at room temperature and in ambient air. The current density-voltage (J-V) characteristics were measured in both forward and reverse scans from 1.2 V to −0.5 V with a scan rate of 50 mV s⁻¹. Max power point tracking (MPPT) algorithm was utilized to ascertain stabilized power output of 120 seconds. Masking was also adopted to define cell area of 0.0625 cm² within the active device area of 0.128 cm². During the characterization, N₂ gas flow was introduced, and temperature was not regulated. All devices were measured without pre-conditioning such as light-soaking and applied bias voltage.

For the long-term thermal stability measurement, a Fluxim Litos, a stress-test platform for degradation analysis, was employed. The solar cells were stressed at temperature of 65° C. in N₂ rich environment, under 1 sun equivalent illumination without UV and continuous MPP tracking. This follows the International Summit on Organic PV Stability (ISOS) L-2I protocols for stability measurement; L, 2, and I stand for light on with bias, high temperature, and inert atmosphere, respectively.³ During the stability measurement, automated acquisition of J-V scans in both reverse and forward directions were taken every 12 hours.

TABLE 1
Elemental composition analysis of the TPT
polymers to ensure purity of the samples.

| Polymer | Theoretical composition | | Measured Composition | |
|---|---|---|---|---|
| TPT-TT | C: 73.47%, | H: 9.03%, | C: 72.84%, | H: 9.02%, |
| | S: 14.01% | | S: 14.40% | |
| TPT-T | C: 75.47%, | H: 9.62%, | C: 74.77%, | H: 9.58%, |
| | S: 11.19% | | S: 10.94% | |
| TPT-T(MB/C6) | C: 72.46%, | H: 8.21%, | C: 71.89%, | H: 8.07%, |
| | S: 14.51% | | S: 14.25% | |

TABLE 2
Optoelectronic properties of CsFA perovskite and HTLs determined
by combination of UV-Vis and UPS measurements.

| | Optical | | Valence | |
|---|---|---|---|---|
| | Energy | Work | Band | Calculated |
| | Band | Function | Maximum | Conduction |
| Thin Film | Gap (eV) | (eV) | (eV) | Band (eV) |
| Cs_{0.09}FA_{0.91}PbI₃ | 1.54 | 4.32 | 5.75 | 4.21 |
| Spiro- | 3.02 | 3.96 | 5.02 | 2.00 |
| OMeTAD | | | | |
| Doped Spiro- | 3.01 | 4.47 | 5.27 | 2.26 |
| OMeTAD | | | | |
| TPT-TT | 2.10 | 4.12 | 4.84 | 2.74 |
| TPT-T | 2.10 | 4.09 | 4.77 | 2.67 |
| TPT-T | 2.16 | 3.96 | 4.72 | 2.56 |
| (MB/C6) | | | | |

TABLE 3
Summarized photovoltaic parameters extracted from
the reverse J-V curves of PSCs from FIGS. 12-12F.

| HTL | REF | TPT-TT | TPT-T | TPT-TT (MB/C6) |
|---|---|---|---|---|
| Max J_{SC} (mA cm⁻²) | 25.1 | 22.37 | 23.5 | 23.99 |
| Median J_{SC} (mA cm⁻²) | 24.66 | 21.68 | 22.6 | 23.48 |
| Max V_{OC} (V) | 1.06 | 0.94 | 0.94 | 0.94 |
| Median V_{OC} (V) | 1.03 | 0.89 | 0.9 | 0.93 |
| Max FF (%) | 81.31 | 40.61 | 48.71 | 51.17 |
| Median FF (%) | 75.96 | 36.15 | 45.41 | 48.06 |
| Max PCE (%) | 20.29 | 7.59 | 10.56 | 10.94 |
| Median PCE (%) | 19.4 | 6.96 | 9.38 | 10.43 |
| Max Stabilized PCE (%) | 20.15 | 7.39 | 10.04 | 11.09 |
| Median Stabilized PCE (%) | 19.13 | 6.76 | 8.36 | 10.04 |

TABLE 4
Summarized photovoltaic parameters extracted from
the reverse J-V curves of PSCs from FIGS. 16A-16F.

| | | TPT-TT | TPT-TT (MB/C6) + |
|---|---|---|---|
| HTL | REF | (MB/C6) | 1.2M Li-TFSI |
| Max J_{SC} (mA cm⁻²) | 24.97 | 24.34 | 24.42 |
| Median J_{SC} (mA cm⁻²) | 24.6 | 23 | 24.06 |
| Max V_{OC} (V) | 1.03 | 0.96 | 0.98 |
| Median V_{OC} (V) | 0.99 | 0.9 | 0.92 |
| Max FF (%) | 80.65 | 60.87 | 65.96 |
| Median FF (%) | 76.56 | 55.01 | 61.99 |
| Max PCE (%) | 19.65 | 13.21 | 15.65 |
| Median PCE (%) | 18.32 | 11.54 | 13.45 |
| Max Stabilized PCE (%) | 19.00 | 12.44 | 12.53 |
| Median Stabilized PCE (%) | 17.31 | 10.06 | 8.36 |

### REFERENCES FOR SUPPLEMENTARY EXPERIMENTAL DETAILS FOR EXAMPLE 1

(1) Sabury, S.; Xu, Z.; Saiev, S.; Davies, D.; Osterholm, A. M.; Rinehart, J. M.; Mirhosseini, M.; Tong, B.; Kim, S.; Correa-Baena, J. P.; Coropceanu, V.; Jurchescu, O. D.; Brédas, J. L.; Diao, Y; Reynolds, J. R. Non-Covalent Planarizing Interactions Yield Highly Ordered and Thermotropic Liquid Crystalline Conjugated Polymers. Mater Horiz 2024, 11 (14), 3352-3363.
(2) Endres, J.; Egger, D. A.; Kulbak, M.; Kerner, R. A.; Zhao, L.; Silver, S. H.; Hodes, G.; Rand, B. P.; Cahen, D.; Kronik, L.; Kahn, A. Valence and Conduction Band Densities of States of Metal Halide Perovskites: A Combined Experimental-Theoretical Study. J Phys Chem Lett 2016, 7 (14), 2722-2729.
(3) Khenkin, M. V.; Katz, E. A.; Abate, A.; Bardizza, G.; Berry, J. J.; Brabec, C.; Brunetti, F.; Bulović, V; Burlingame, Q.; Di Carlo, A.; Cheacharoen, R.; Cheng, Y. B.; Colsmann, A.; Cros, S.; Domanski, K.; Dusza, M.; Fell, C. J.; Forrest, S. R.; Galagan, Y; Di Girolamo, D.; Grätzel, M.; Hagfeldt, A.; von Hauff, E.; Hoppe, H.; Kettle, J.; Köbler, H.; Leite, M. S.; Liu, S. (Frank); Loo, Y. L.; Luther, J. M.; Ma, C. Q.; Madsen, M.; Manceau, M.; Matheron, M.; McGehee, M.; Meitzner, R.; Nazeeruddin, M. K.; Nogueira, A. F.; Odaba i, Q.; Osherov, A.; Park, N. G.; Reese, M. O.; De Rossi, F.; Saliba, M.; Schubert, U. S.; Snaith, H. J.; Stranks, S. D.; Tress, W.; Troshin, P. A.; Turkovic, V; Veenstra, S.; Visoly-Fisher, I.; Walsh, A.; Watson, T.; Xie, H.; Yildirim, R.; Zakeeruddin, S. M.; Zhu, K.; Lira-Cantu, M. Consensus Statement for Stability Assessment and Reporting for Perovskite Photovoltaics Based on ISOS Procedures. Nature Energy 2020 5:1 2020, 5 (1), 35-49.

### Example 2. Non-Covalent Planarizing Interactions Yield Highly Ordered and Thermotropic Liquid Crystalline Conjugated Polymers

Controlling the multi-level assembly and morphological properties of conjugated polymers through structural manipulation has contributed significantly to the advancement of organic electronics. In this example, a redox active conjugated polymer, TPT-TT, composed of alternating 1,4-(2-thienyl)-2,5-dialkoxyphenylene (TPT) and thienothiophene (TT) units is reported with non-covalent intramolecular S . . . O and S . . . H—C interactions that induce controlled main-chain planarity and solid-state order. As confirmed by density functional theory (DFT) calculations, these intramolecular interactions influence the main chain conformation, promoting backbone planarization, while still allowing dihedral rotations at higher kinetic energies (higher temperature), and give rise to temperature-dependent aggregation properties. Thermotropic liquid crystalline (LC) behavior is confirmed by cross-polarized optical microscopy (CPOM) and closely correlated with multiple thermal transitions observed by differential scanning calorimetry (DSC). This LC behavior allows us to develop and utilize a thermal annealing treatment that results in thin films with notable long-range order, as shown by grazing-incidence X-ray diffraction (GIXD). Specifically, we identified a first LC phase, ranging from 218° C. to 107° C., as a nematic phase featuring preferential face-on π-π stacking and edge-on lamellar stacking exhibiting a large extent of disorder and broad orientation distribution. A second LC phase is observed from 107° C. to 48° C., as a smectic A phase featuring sharp, highly ordered out-of-plane lamellar stacking features and sharp tilted backbone stacking peaks, while the structure of a third LC phase with a transition at 48° C. remains unclear, but resembles that of the solid state at ambient temperature. Furthermore, the significance of thermal annealing is evident in the ˜3-fold enhancement of the electrical conductivity of ferric tosylate-doped annealed films reaching 55 S cm⁻¹. More importantly, thermally annealed TPT-TT films exhibit both a narrow distribution of charge-carrier mobilities (1.4±0.1)×10⁻² cm² V⁻¹ s⁻¹ along with a remarkable device yield of 100% in an organic field-effect transistor (OFET) configuration. This molecular design approach to obtain highly ordered conjugated polymers in the solid state affords a deeper understanding of how intramolecular interactions and repeat-unit symmetry impact liquid crystallinity, solution aggregation, solution to solid-state transformation, solid-state morphology, and ultimately device applications.

The macromolecular assembly of conjugated polymers is determined by π-π stacking, main-chain planarity, side-chain structure, and placement, regioregularity, and symmetry. In this example, we have designed a conjugated polymer with regiosymmetric repeat units that we intentionally engineer to enable intramolecular through-space interactions along the backbone. The novelty of our design concept resides in its seeking to serve simultaneously the following purposes: (1) regulating the polymer backbone's conformation, (2) managing solubility and aggregation behavior, and (3) creating a highly organized and ordered solid-state structure. While there exists a considerable body of literature detailing the integration of non-covalent interactions into the structural framework of conjugated materials, both small molecules and polymers, the primary focus has typically been on inducing planarity without the solubility challenges associated with other planarization strategies, such as ladder-type designs. To date, the investigation of the reversibility of these non-covalent intramolecular interactions in the solid state has received significantly less attention. For instance, to the best of our knowledge, there are no reports concerning the utilization of intramolecular reversible conformational locks to induce liquid crystallinity. Thus, the present work contributes significantly to the fundamental design principles aimed at achieving heightened control over the self-assembly of conjugated polymers.

### Introduction

Polymers that assemble into ordered macromolecular structures have greatly contributed to the advancement of organic electronics, including the development of highly performing photovoltaic devices, light-emitting diodes, and field-effect transistors, as well as the creation of highly conductive and electroactive materials (1-4). The long-range order in conjugated polymers, described as the extended spatial coherence of the polymer chains over considerable distances, contributes significantly to the electronic and optical properties of these materials. Within the ordered regions, the extended π-electron conjugation and minimal energetic disorder substantially reduces energy barriers for charge transport, resulting in improved energy and charge transfer properties (5-8). Hence, achieving long-range order in conjugated polymers through precise molecular design is an important goal as it optimizes their performance in (opto)electronic devices.

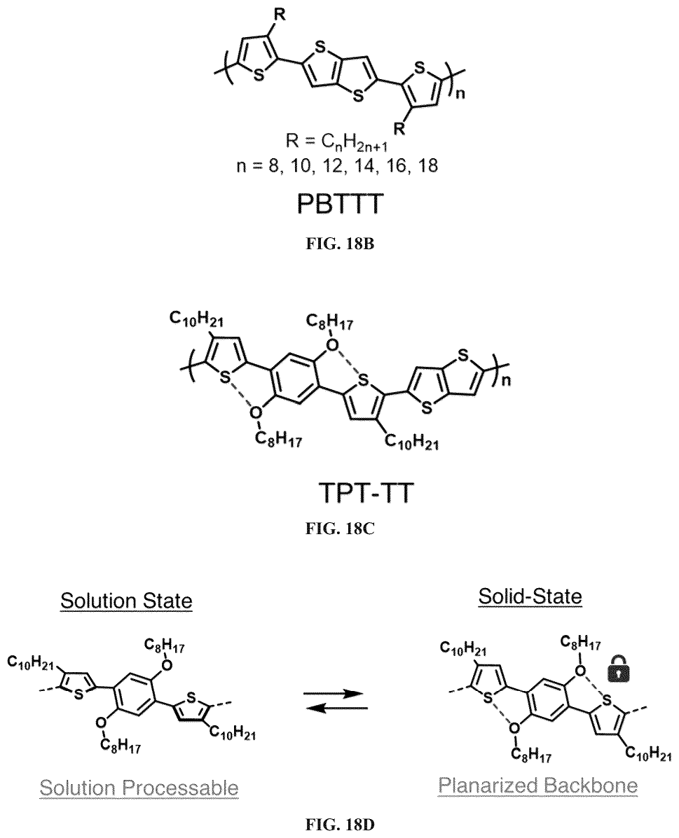

Backbone π-π stacking and chain planarity, as well as side-chain length and bulkiness, regioregularity, and symmetry are recognized as primary factors controlling chain organization in conjugated polymers (1,3). Regioregularity, as exemplified by poly(3-hexylthiophene) (P3HT), plays a crucial role in promoting the formation of well-ordered and semicrystalline polymer structures as the material transitions from a solution phase to the solid state (9,10). Moreover, using symmetrically substituted monomers to form polymers with regiosymmetric repeat units minimizes chain defects and promotes longer-range ordering (11-14). For instance, poly(1,4-bis(2-thienyl)phenylene) (TPT) with symmetrical bis-dodecyloxy side-chains (—OC₁₂H₂₅, —OC₁₂H₂₅) is highly crystalline, whereas the asymmetric homolog with unsymmetrical substituent placement (—OCH₃, —OC₁₂H₂₅) has an amorphous solid-state morphology (12). In addition to structures similar to poly(1,4-bis(2-thienyl)phenylene) (15,16), regiosymmetry in dioxythiophene-based and other high-mobility conjugated polymers is also shown to promote ordered chain packing (14,17,18). Conversely, repeat unit asymmetry reduces the molecular packing tendency and aggregation behaviors in both solid and solution states (19,20). Various approaches such as the incorporation of fused rings (21), ladder-type structures (22), as well as non-covalent intramolecular interactions (23) have been taken to promote backbone planarity to improve the solid-state order in conjugated polymers along with making torsion-free highly performing amorphous conjugated polymers (24-26). While fused ring and ladder-type structures often impose solubility limitations, backbone planarization through non-covalent interactions (such as S . . . O and S . . . F) leads to highly planar yet solution processable polymers (23, 27-30). Non-covalent interactions impacting oxidation potential and conductivity in doped complexes are exemplified in the poly(3,4-ethylenedioxythiophene)'s (PEDOT, FIG. 18A) rigid and co-planar backbone and attributed to the through-space short contacts between neighboring sulfur and oxygen atoms (31,32). This conformational control through non-covalent heteroatom interactions notably influences, e.g., the transport gap (corresponding to the difference between the polymer ionization potential and electron affinity or, roughly speaking, to the energy gap between the highest-occupied molecular orbitals (HOMO) and the lowest unoccupied molecular orbitals (LUMO)). Variations in the transport gap as large as ˜1 eV have been correlated to this effect (32). Due to the reversible formation-dissociation nature of these non-bonding interactions (33-36), the resulting polymers retain solution processability (23). Once processed, these non-covalent interactions gain greater significance in the solid state as they promote the formation of more ordered structures through conformational locking (37).

In addition to regiosymmetry, regioregularity, and backbone planarity, side-chain intercalation and crystallization also contribute to the solid-state morphology and order (38-40). For example, regiosymmetric poly[2,5-bis(3-alkylthiophen-2-yl)thieno[3,2-b]thiophene](PBTTT, FIG. 18B) exhibits high hole mobility (on the order of 0.1-1 cm² V⁻¹ s⁻¹), high conductivity (exceeding 400 S cm⁻¹) upon chemical doping, as well as liquid crystalline behavior owing to its distinctive side-chain interdigitation (41-43). While the as-cast PBTTT-C16 (PBTTT with hexadecyl side-chain, n=16 in FIG. 18B) has moderate mobilities ˜0.069 cm² V⁻¹ s⁻¹, further post-processing treatments such as annealing and high-temperature rubbing leads to mobilities as high as ˜1 cm² V⁻¹ s⁻¹ owing to highly ordered morphologies in optimized transistor devices (44,45). Likewise, significant enhancement in the electrical conductivity of the sequentially doped PBTTT films has been reported following post-processing treatments (46,47). These performance improvements stemming from post-processing treatments are attributed to a high degree of crystallinity and/or alignment brought by thermotropic liquid crystallinity that improves interchain transport.

Here, our aim was to create a thermotropic, liquid crystalline, redox active material with the potential to form highly ordered structures. To achieve this, we combined the regiosymmetric 1,4-(2-thienyl)-2,5-dialkoxyphenylene (TPT) with two non-covalent interactions per unit (S . . . O and S . . . H—C) with thienothiophene (TT) as the π-bridge to obtain TPT-TT (FIG. 18C). FIG. 18D illustrates how the non-bonding interactions allow this polymer to switch between a torsionally flexible form and a conformationally locked state that dictates the chain arrangement. Photophysical measurements show temperature-dependent formation of polymer aggregates in solution, whereas multiple thermal transitions (observed by differential scanning calorimetry, DSC) and the birefringent optical response confirm liquid crystalline behavior. This liquid crystalline behavior is then exploited to further enhance the solid-state order by thermally annealing the thin films, resulting in diffraction patterns indicating long-range order through in situ grazing incidence X-ray diffraction (GIXD) measurements. Ordered phase unit-cell determination demonstrates that side-chain interdigitation during the annealing process boosts the coherent solid-state ordering. The impact of enhanced solid-state order upon annealing is exemplified by the improvement in the electrical conductivity (from 18 to 55 S cm 1) in ferric tosylate doped TPT-TT films. Field-effect transistors with TPT-TT as the transport layer material exhibited hole mobilities of (1.4±0.1)×10⁻² cm² V⁻¹ s⁻¹. More importantly, the narrow distribution of the measured field-effect mobility values and the high device yield (100%) are particularly promising in the context of device applications.

### Results and Discussion

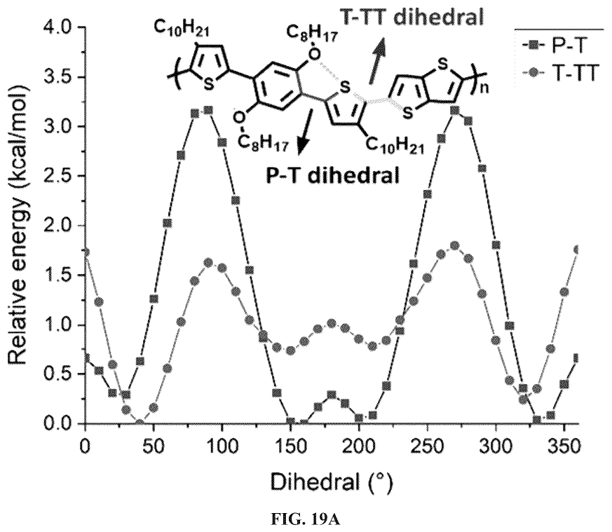

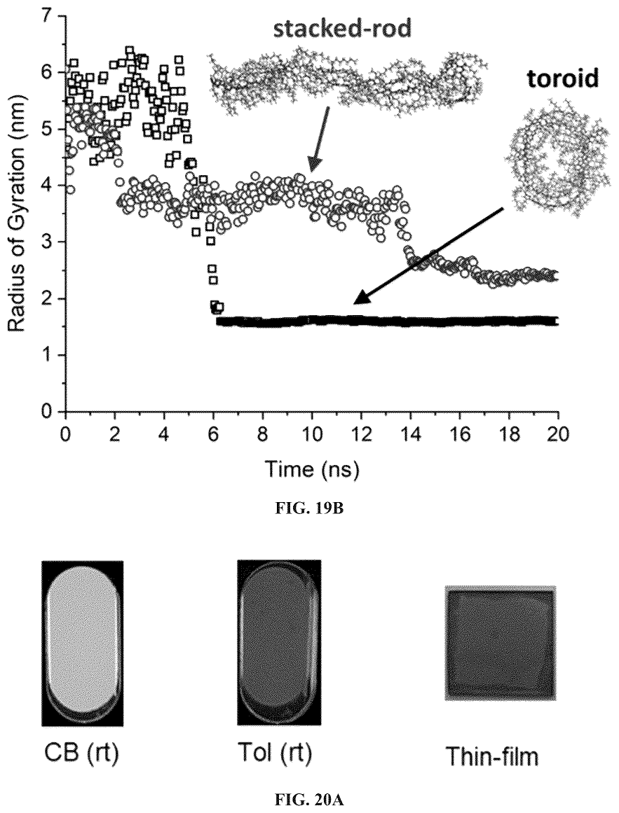

To investigate the impact of intra-chain interactions on backbone conformation, we carried out density functional theory (DFT) calculations to examine the dihedral potentials along the TPT-TT repeat unit in isolated chains, i.e., in the “gas phase” (see FIG. 19A). The reference energy, set at 0 kcal mol⁻¹, corresponds to the anti-conformation of the T-TT group (as illustrated by the green bonds in the inset of FIG. 19A). We note that the alkyl side-chains were modeled by ethyl groups, an approach that effectively circumvents the computational complexities associated with longer chains while enabling the assessment of the influence of side-chain bulkiness on torsional barriers. As evidenced by the low-energy conformers at 150°-200° in the phenylene-thienylene (P-T) dihedral (purple bonds in the inset of FIG. 19A, 0° representing anti-conformation for oxygen and sulfur in P-T bond), the through-space non-covalent S . . . O interactions play a determining factor in inducing backbone planarity. Stabilization of this dihedral is also seen at approximately 300 and 330°, which is attributed to S . . . H—C interactions. These two sets of minima display comparable energy levels and are separated by an energy barrier ΔE₁=3.17 kcal mol⁻¹, which points to the strong contributions of the intra-chain interactions in the stabilization of the optimal conformations. The rotation of thienylene-thienothiophene (T-TT) is less restricted, with a significantly smaller anti to syn rotational barrier of ΔE₂=1.63 kcal mol⁻¹, making this transition more energetically accessible. It is worth noting that the stabilized T-TT conformations yield a slight reduction in the ring coplanarity (˜40°) compared to P-T conformer. With lower energy barriers and a capacity for wider twist angles, this (T-TT) dihedral angle is expected to facilitate chain coiling.

In addition to the segmental planarity induced by intramolecular contacts, π-π stacking interactions of the constituent rings contribute to the backbone configuration. Specifically, thienothiophene units with their fused-ring structure (and no side-chains) are reported to promote interchain interactions (40,48,49). Our molecular dynamics simulations conducted at 300 K in the gas phase, employing the NVT (constant number of particles, volume, and temperature) ensemble, point to the folding of a single chain. We observe the transitions from linear rod conformations to coiled stacked-rod and toroid structures (FIG. 19B) an evolution driven by the preference to maximize π-π stacking in combination with the nature of the dihedral potential energy surfaces.

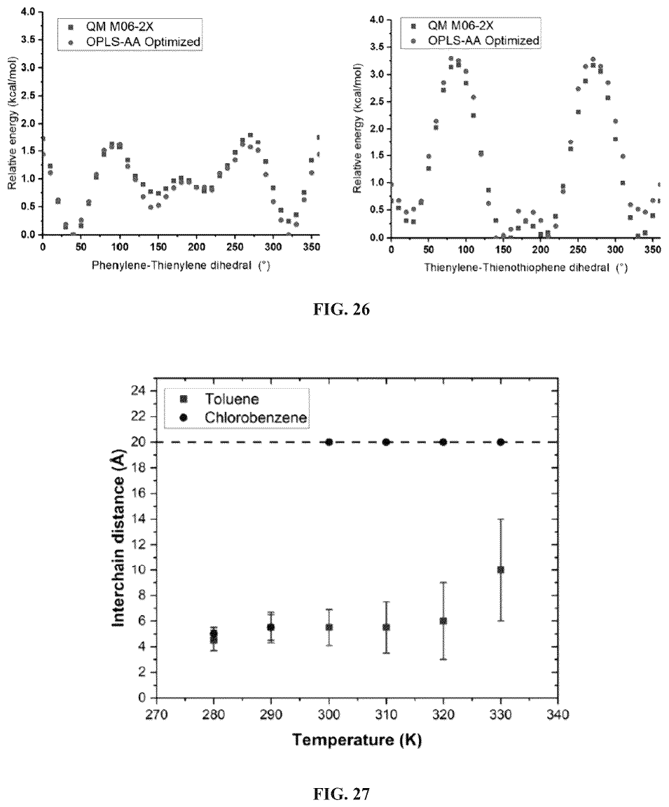

Expanding on the single-chain simulations, the solvation behavior of the TPT-TT polymer was investigated in toluene and chlorobenzene using a pair of trimeric chains (dihedral scans for optimized potentials for liquid simulations—all atom, OPLS-AA, force field is presented in FIG. 26) at a concentration of 15 mg mL⁻¹ to closely mimic the experimental solubility values. Starting from chains being in close vicinity of each other (about 5 Å apart), as shown in FIG. 27, simulations ran for 150 ns or until the centers of mass were separated by 20 Å, a criterion established to indicate irreversible separation. The results for simulations conducted between 280 and 330 K reveal pronounced separation of the chains in chlorobenzene, even at room temperature. In marked contrast, in toluene, the chains maintain their association throughout the simulation, although there are some indications of chain de-aggregation above 320 K. As we will show, these results are consistent with the temperature- and solvent-dependent aggregation tendencies observed for TPT-TT. Overall, the MD simulations indicate the potential for the TPT-TT polymer to exhibit an ordered assembly due to the interplay between chain planarity and π-π stacking, promoted by non-covalent interactions and the thienothiophene unit, respectively. The reversible nature of the non-covalent intra-chain interactions points to the microstructural tunability that TPT-TT could display.

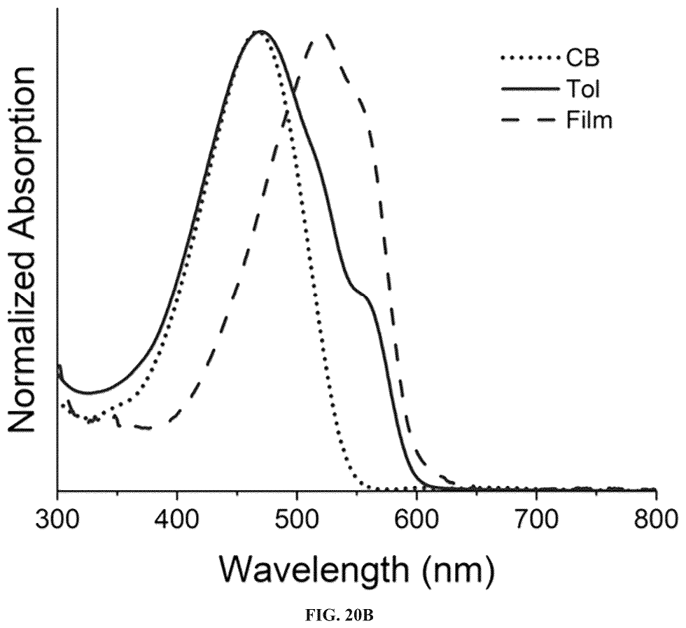

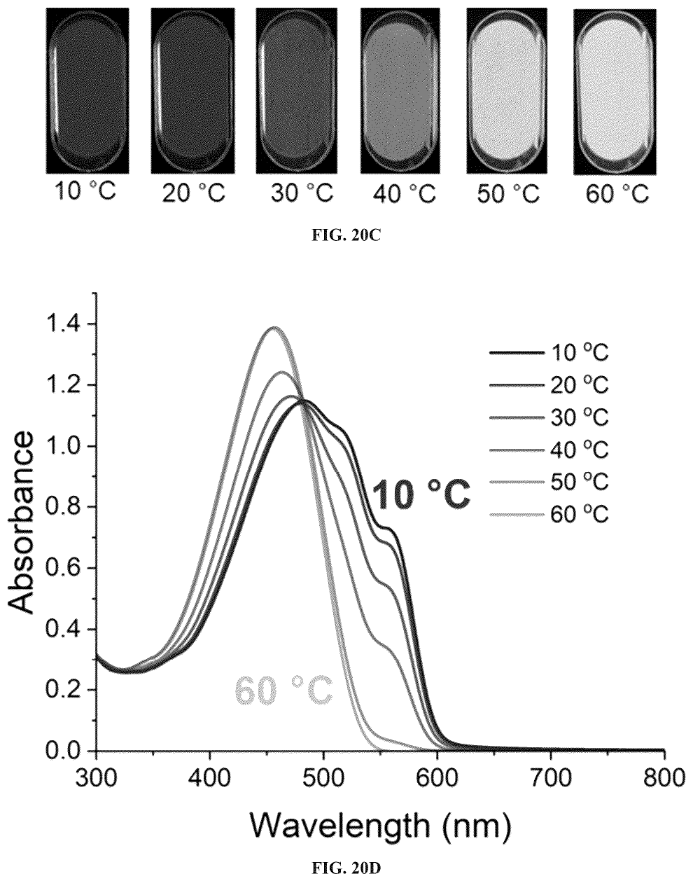

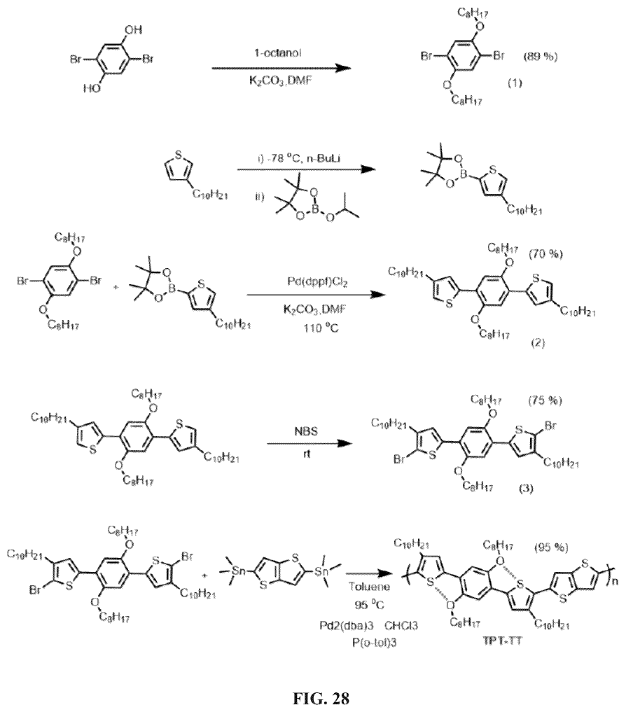

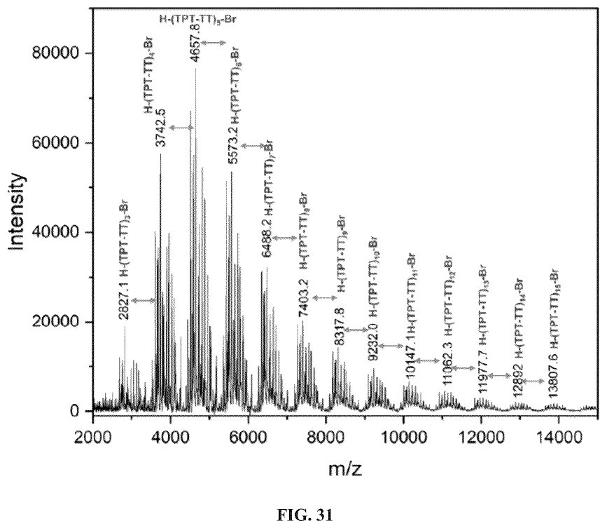

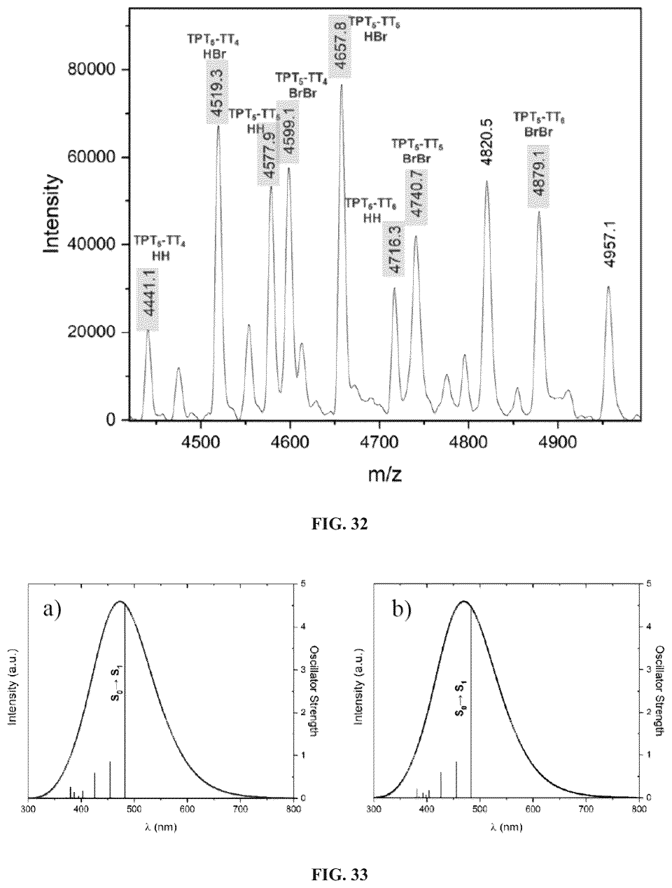

TPT-TT (FIG. 18C), with octyloxy and decyl sidechains attached to the phenyl and thiophene rings, respectively, was synthesized via Stille cross-coupling polymerization (synthetic and general characterization details found below, FIGS. 28-30). The polymer has a number-average molecular weight of 24.6 kg mol⁻¹ and a dispersity (Ð) of 1.93 with a monomodal distribution. Based on matrix-assisted laser desorption ionization (MALDI) time-of-flight (TOF) mass spectrometry, the incorporation ratio of TPT vs. TT units was equivalent, as expected for an alternating co-polymer. There is no evidence of homocoupling, as shown in FIG. 31, and the end group analysis in FIG. 32 shows the expected H—H, Br—Br, and H—Br chain ends. The photographs of polymer solutions and normalized UV-vis absorption spectra in FIG. 20A-20B demonstrate that TPT-TT is well solvated in dilute (˜0.1 mg mL⁻¹) room-temperature chlorobenzene, based on the featureless band with λₘₐₓ of 470 nm (2.64 eV). However, when dissolved in toluene, two low-energy shoulders appear around 520 nm and 558 nm, which are expected to originate from the presence of aggregates or strong intermolecular interactions between polymer segments (50-55). Time-dependent density functional theory (TD-DFT) calculations, based on a TPT-TT trimer and considering implicit solvents, indicate that the main absorption band of a polymer chain in both chlorobenzene and toluene is predominantly due to the S₀→S₁ electronic transition, corresponding to =482 nm (2.56 eV), which closely aligns with the experimentally determined value (see FIG. 33).

The thin-film UV-vis absorption spectrum (films being spin coated from ˜5 mg mL⁻¹ chlorobenzene solution) is red-shifted (onset of absorption of 605 nm) compared to solution and shows two transitions around 522 nm and 558 nm, which resemble a profile similar to that of the aggregated species in toluene. The strong thermochromism (FIGS. 20C-20D) and the solution aggregation behavior highlight how modulation of the planarizing intramolecular non-covalent contact using solvent quality (toluene vs. chlorobenzene) or temperature dictates the chain organization, changing from a highly aggregated solution at 10° C. to a well-dissolved solution at 50° C. in toluene.

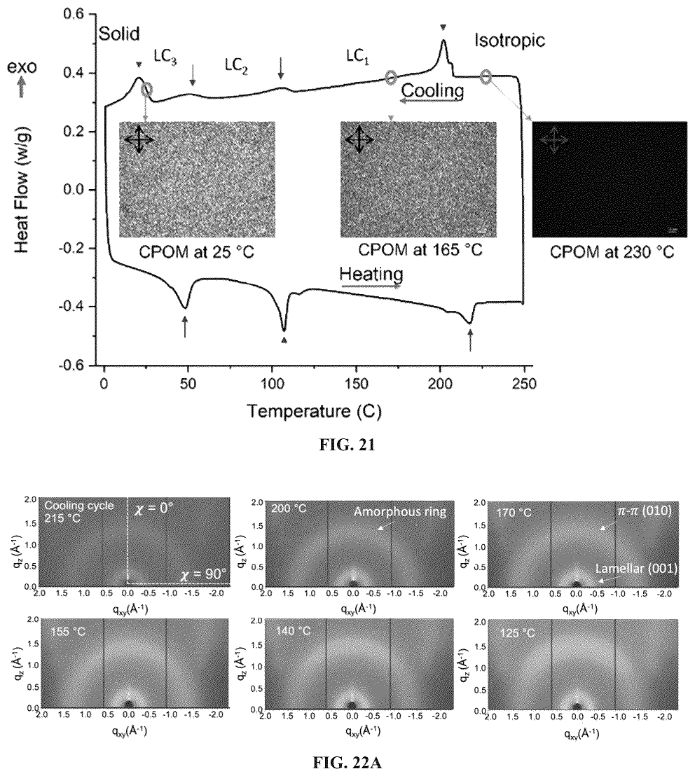

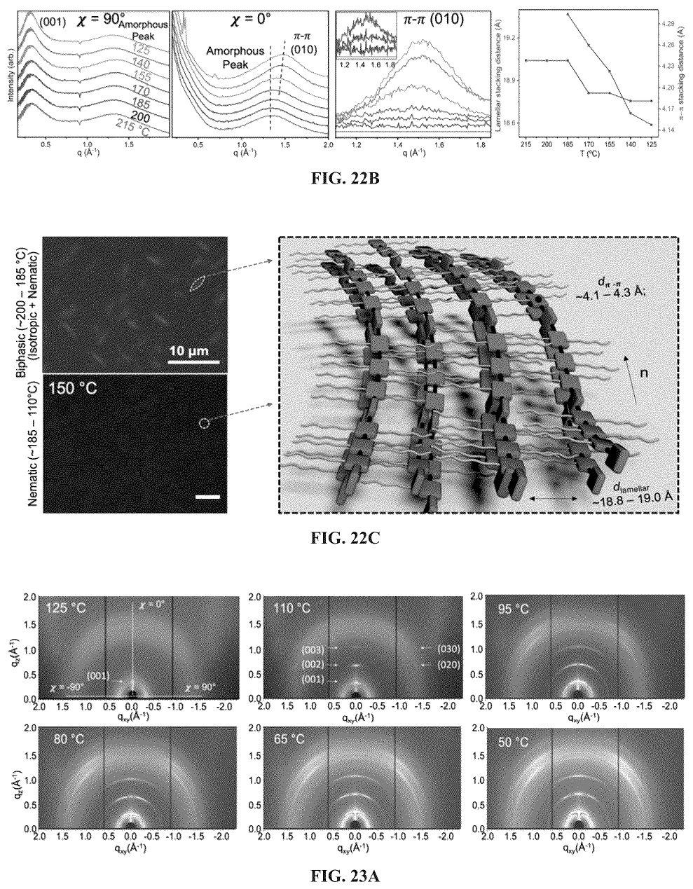

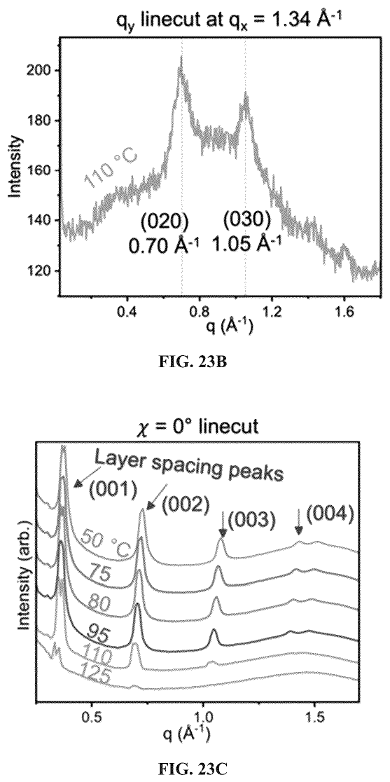

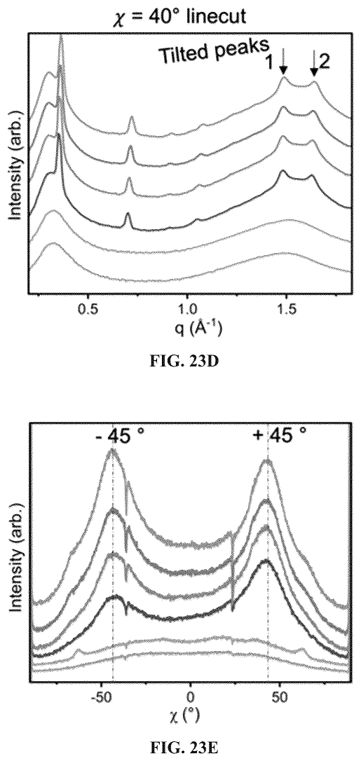

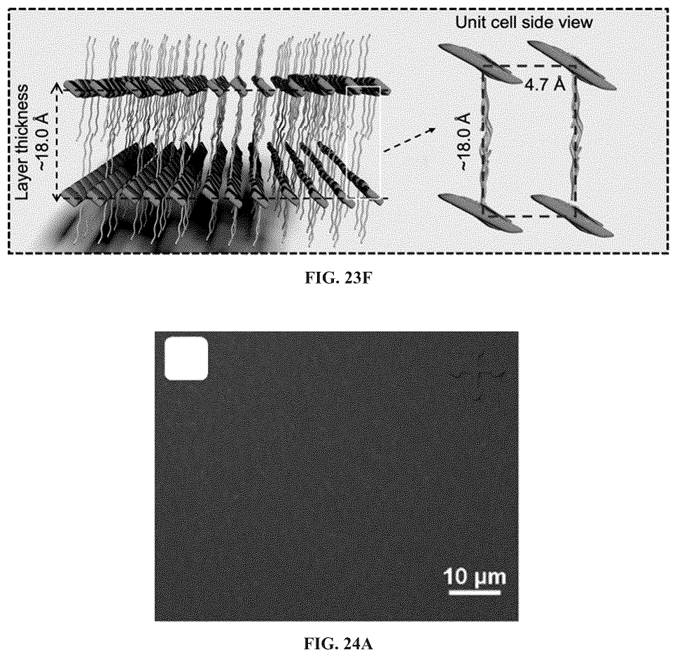

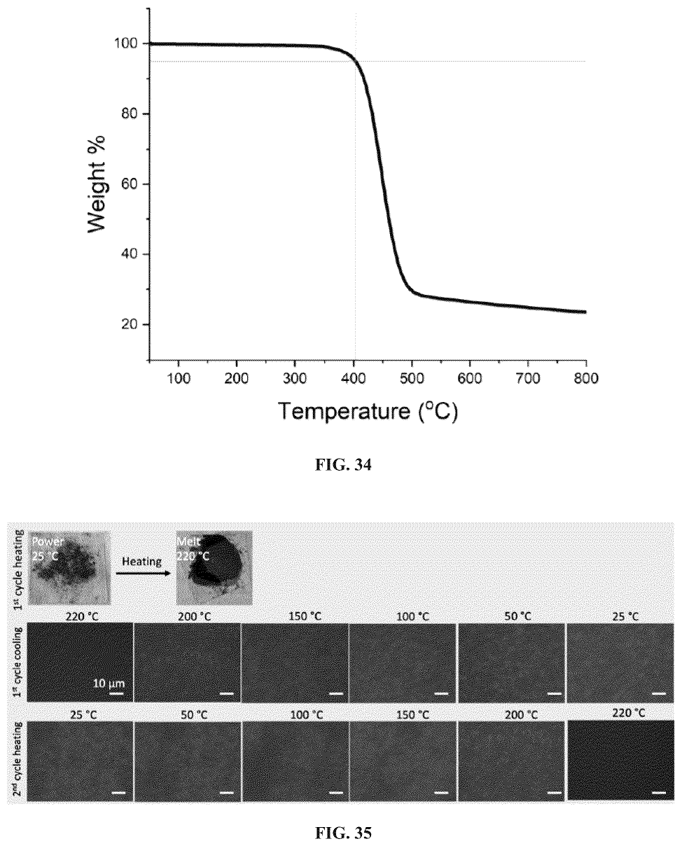

Subsequently, we evaluated if these temperature- and solvent-dependent structural locking interactions can lead to multi-phase ordered assemblies. We note (FIG. 34) that TPT-TT is thermally stable, and the onset of the decomposition process (405° C. at 5% weight loss) is well above the thermal transitions observed by DSC. The DSC scan of TPT-TT (FIG. 21) shows three well-defined endotherms in the second heating cycle, occurring at 48° C., 107° C., and 218° C., indicative of liquid crystalline (LC) behavior with multiple phases. The thermal transitions at 107° C. and 218° C. are assigned to side-chain and backbone order-disorder transitions, respectively, based on our GIXD analysis (see below) and previous literature reports (44,48,56,57). The thermotropic liquid crystalline behavior of TPT-TT was directly observed using cross-polarized optical microscopy (CPOM) for thin films (FIG. 23, insets) and for polymer powders (FIG. 35). When heating to temperatures above the 218° C. transition, which results in a molten isotropic state, the sample appears dark under CPOM. Subsequent LC-LC transitions between 150° C. and 25° C. did not show appreciative changes under CPOM possibly due to the subtlety of the structural changes that did not lead to significant refractive index change. These thermal transitions are reversible, as evidenced by the cooling cycle in DSC.

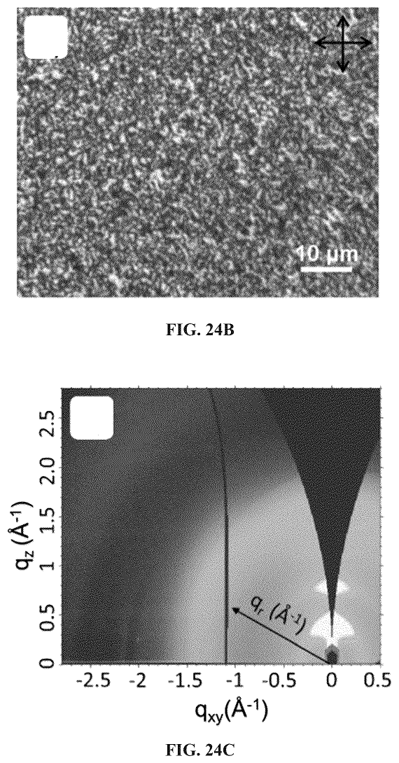

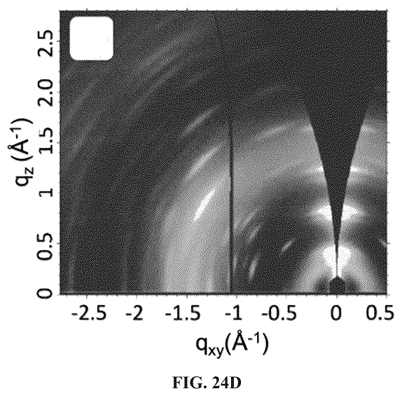

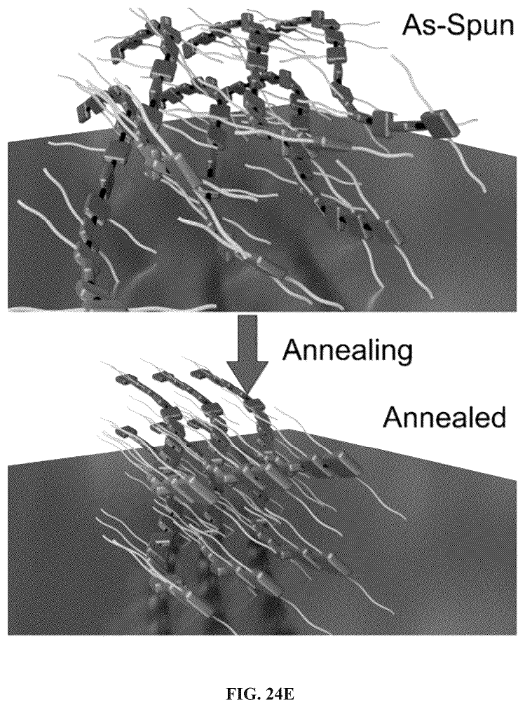

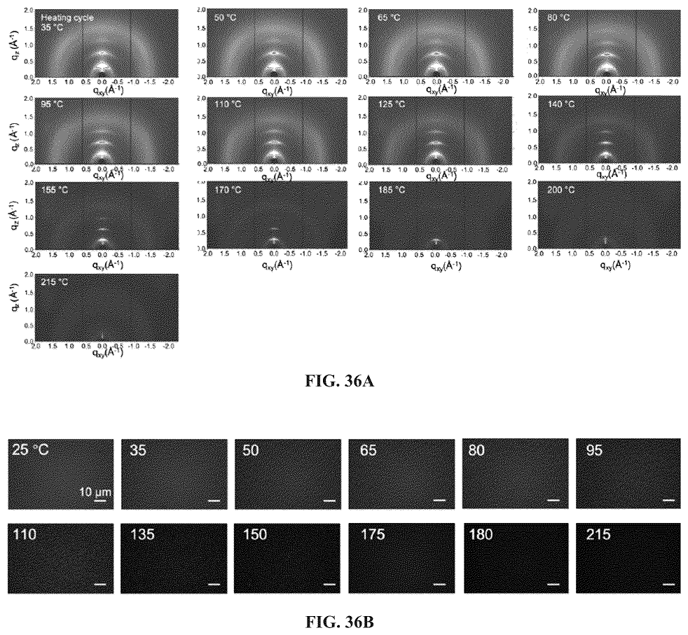

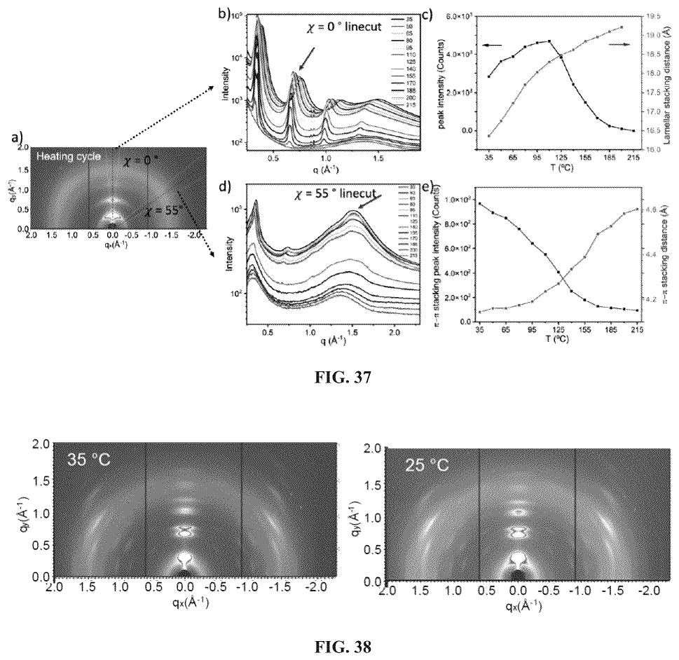

To elucidate the structural basis to the phase transitions observed in DSC, we performed synchrotron-based GIXD to investigate the evolution of molecular-scale packing structures in spin-coated films during thermal annealing. Initial observations during the first heating cycle revealed that the crystals formed during spin-coating underwent melting, a process captured in both GIXD and CPOM images (FIGS. 36A-36B). This crystal melting is supported by the observed expansion of the lamellar and π-stacking distances, from 16.36 to 19.21 Å and 4.14 to 4.60 Å respectively (FIG. 37). Additionally, the intensity of π-stacking peaks, observed both face-on (χ=0°) and at a χ=650 azimuthal tilt, diminishes progressively with increasing temperature. Intriguingly, the intensity of the lamellar stacking peak exhibits a notable increase from 35° C. to 110° C. before dropping to zero as temperatures reach from 110° C. to 215° C., accompanied by substantial peak broadening over the entire temperature range. This behavior is indicative of a phase transition near 110° C., consistent with DSC. We surmise that the structural transition near 110° C. arise from side-chain melting. This inference aligns with findings from a thiophene-based polymer PBTTT-C14, which possesses long alkyl side-chains of 14 carbons and exhibits a side-chain melting temperature of 150° C. (57,58). We note that the absence of structural transitions other than melting during the first heating run may be due to the fact that the spin-coated films are originally entrapped in non-equilibrium phases. Therefore, transitions from the stable structure at room temperature were not observable upon initial heating. This point is well supported by our observation that the film structures are drastically different before and after thermal annealing (FIGS. 24A-24E).

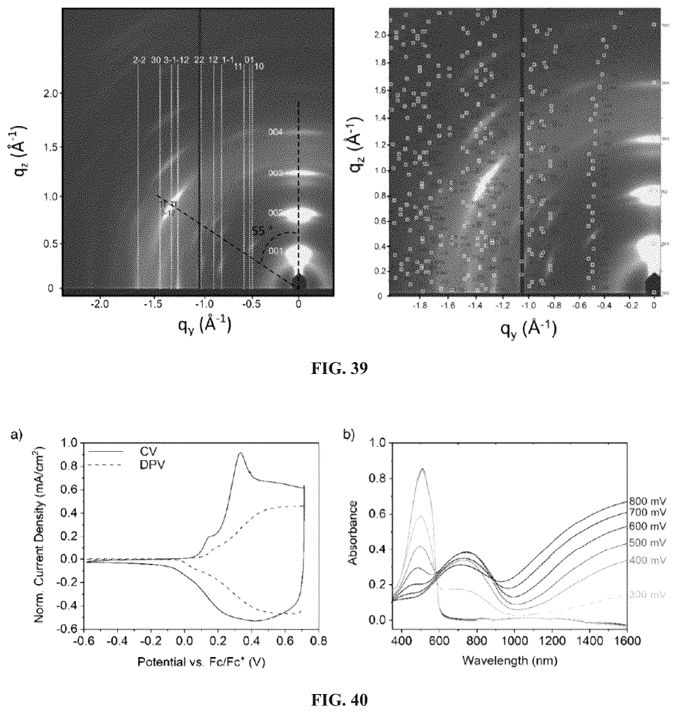

Upon further cooling from 125° C. to 50° C., we identified LC phase II as a Smectic A phase. At 125° C., a new (001) lamellar stacking peak appears in the out-of-plane direction at ˜0.35 Å⁻¹. Upon further cooling to 110° C. near the DSC transition temperature of 107° C., there emerged second and third lamellar stacking peaks at 0.70 Å and 1.05 Å⁻¹, with a fourth-order peak becoming apparent at q_{z}=1.40 Å⁻¹ when cooled to 95° C. (FIG. 23A). These peaks are assigned as (002), (003), and (004) respectively. These sharp out-of-plane lamellar stacking peaks are distinct from the broad lamellar stacking peak from the nematic LC phase I centered around 0.33 Å⁻¹ and oriented in-plane. The new lamellar stacking peaks are characterized by much narrower full-width-half-maximum (FWHM) values of 0.02 Å⁻¹, in contrast to 0.15 Å⁻¹ for the nematic lamellar stacking peak. These observations suggest that the new lamellar stacking feature has alkyl chains standing on the substrate rather than lying flat, possesses closer lamellar stacking distance, and is far more ordered than that of the nematic phase. These features suggest the formation of a highly ordered smectic-like structure with a layer spacing of ˜18.0 Å in the out-of-plane direction (65). Besides the new out-of-plane peaks, at 110° C. two sharp peaks appear at q_{xy}=±1.35 Å⁻¹ forming a Bragg rod, which we infer to be originated from π-π stacking of tilted polymer backbones and assigned as (020) and (030) respectively. The lowest order (010) peak is likely buried by background scattering due to the overlapping amorphous ring. This inference is based on how the two peaks further evolve during cooling all the way until crystallization near room temperature. The inference is consistent with the peak indexing result for annealed crystalline thin films discussed below (FIGS. 24A-24E, FIG. 39). Based on the peak assignment, we estimate a π-stacking distance of 4.24 Å calculated based on the qᵣ position of the (010) peak. We note that the (020) and (030) peaks appear along the same q_{z}=0.70 Å⁻¹ and 1.05 Å⁻¹ as the (002) and (003) lamellar stacking peaks, together suggesting an orthorhombic unit cell with alkyl chains standing normal to the substrate, hence the inference of smectic A phase. Further cooling results in spreading of (020) and (030) peaks along azimuthal angles, giving rise to two sharp and intense arcs at qᵣ=1.48 Å⁻¹ and qᵣ=1.63 Å⁻¹, atop the broad nematic π-stacking peak (FIG. 23D). The two arcs appear at similar qᵣ positions as the (020) and (030) peaks respectively suggesting the same structural origin (i.e., π-π stacking). We speculate that such peak spreading from a sharp dot to an arc is due to interfacial nucleation of the smectic A phase followed by growth of this phase into the bulk. A linecut along χ angle (−90° to 90°) at fixed qᵣ=1.48 Å⁻¹ reveals that the backbone stacking peak is tilted at an χ angle of 45° (FIG. 23E). We thus propose a smectic A packing structure in LC II, as illustrated in FIG. 23F: the side-chains are layered up in the out-of-plane direction, forming ordered smectic layers with a thickness of nearly 18.0 Å; the backbones are tilted 45° relative to the layer normal. With further cooling to below the DSC transition temperature at 48° C., we observed that GIXD patterns at 35° C. and 25° C. exhibit numerous sharp diffraction peaks (FIG. 38). These peaks indicate a highly ordered structure obtained in LC phase III and the crystalline thin film. Based on this diffraction pattern, we successfully solved the unit cell structure for annealed thin film, as discussed below.

Since thermotropic LC polymers are responsive to thermal annealing (4,66), due to their ability to align while having fluidic behavior, we evaluated the effect of thermal annealing on the solid-state structure of TPT-TT films. FIGS. 24A-24B present the CPOM images of a spin-coated film before and after thermal annealing across the various LC phases described earlier. The as-spun film displays negligible birefringence under CPOM, suggesting a low degree of crystallinity. In contrast, the annealed film demonstrates a high birefringence, indicating a notable increase in the number and/or size of crystallites (67-69). Furthermore, the CPOM images of the annealed film (FIG. 24B, FIG. 35) reveal Schlieren textures (70,71), further corroborating the liquid-crystalline phase assignments discussed above. The enhanced crystallinity is also confirmed by GIXD measurements. The 2D GIXD pattern of the as-spun film shown in FIG. 24C displays three diffraction peaks along q_{z} at 0.39 Å⁻¹, 0.78 Å⁻¹, and 1.17 Å⁻¹, corresponding to out-of-plane lamellar stacking at spacings closer than those in the smectic A phase. We also observed a broad peak at q_{z}=1.51 Å⁻¹, which we ascribe to a π-stacking distance of 4.16 Å, comparable to that of the nematic phase (4.15 to 4.30 Å). This stacking appears primarily in the face-on orientation, with the conjugated core parallel to the substrate surface. These features suggest that this broad face-on π-stacking is inherited from the nematic phase. There is a weaker peak at q_{xy}=−1.20 Å⁻¹, q_{z}=1.00 Å⁻¹ appearing at comparable qᵣ position (1.56 Å⁻¹) as the face-on π-stacking peak. We believe this peak arises from π-stacking of tilted backbones also likely inherited from the smectic A phase due to the resemblance to the (020) π-stacking arc. Such tiled π-stacks in the as-spun film may have served as the precursor to nucleating highly ordered crystallites during thermal annealing when the same peak significantly intensifies and shifts to a slightly higher q position. In contrast to a few broad diffraction features in as-spun films, numerous sharp diffraction peaks appear (FIG. 24D) after the film undergoes thermal annealing through the liquid crystalline phase (i.e., heating to 250° C. followed by slow cooling to room temperature at which point the GIXD was taken), signifying the formation of highly ordered crystalline structures with long-range order. We observed new lamellar stacking peaks appearing at higher q_{z} positions after annealing. This gives a closer lamellar stacking distance of 14.96 Å after annealing compared to 16.11 Å before annealing, which possibly arises from side chain interdigitation during liquid crystal mediated crystallization during the annealing process.

Unit-cell parameters were obtained from GIXD by minimizing the least square error between the diffraction peak positions simulated from the fitted unit cell and the measured positions (a=14.2 Å, b=13.9 Å, c=16.2 Å, α=71.1°, β=86.8°, γ=64.8°). A detailed description of the fitting process can be found below. We note that certain peaks of the initial structure still persist after annealing, this is noticeable in the out-of-plane lamellar stacking peaks that were initially excluded from the fitting. Based on this fitted unit cell, we identified each of the Bragg rods—vertical diffraction lines with equivalent h and k indices—which characterize the in-plane crystal structure along the a- and b-axes (FIG. 39). We found good agreement between the measured and simulated diffraction patterns. By fitting the unit cell, we can determine the indices of the π-stacking peak as (31̅2), corresponding to a qᵣ of 1.60 Å⁻¹ and a reduced π-stacking distance of 3.97 Å in the annealed film. Compared to the nematic face-on and smectic 45° tilted π-stacking peak, which has spacings of 4.15-4.30 Å and 4.21-4.25 Å, respectively, the (31̅2) peak exhibits a more closely packed and tilted nature, with a tilting angle of 35° with respect to the substrate normal (FIG. 39). Based on this analysis, we developed a schematic showing how the packing and chain alignment changes upon annealing (FIG. 24E). It appears as if the polymer chains in as-spun films are initially deposited in layers of disordered π-stacks with bimodal distribution inherited from both the nematic and smectic A LC phases. Then, upon cooling from the melt state, the polymer backbone initially forms predominantly face-on stacking in the nematic phase. This is followed by the formation of tilted stacking in the smectic A phase, which then transitions to closely spaced π-stacks less tilted with respect to the substrate normal in the solid state. Meanwhile, alkyl chains undergo order-disorder transitions with a higher extent of interdigitation to result in a highly crystalline phase that displays exceptional long-range order unusual for conjugated polymers.

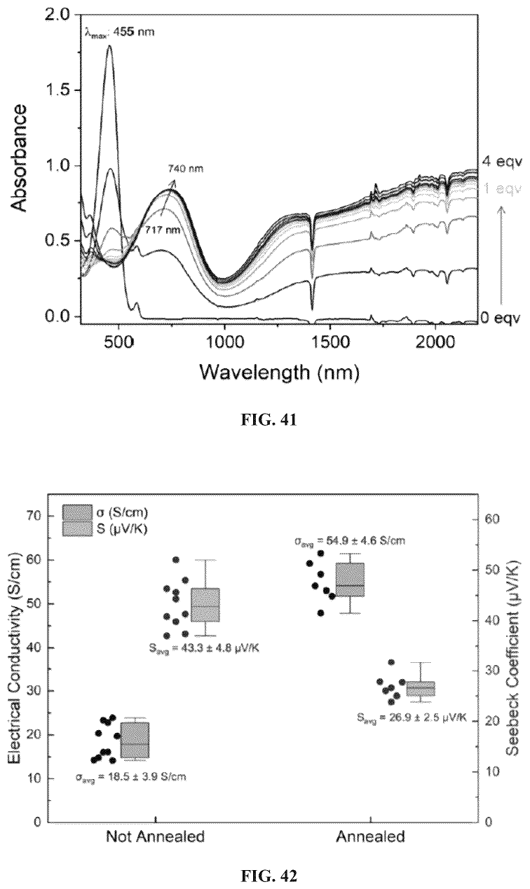

The ionization energy (IE) and doping susceptibility of TPT-TT was estimated by differential pulse voltammetry (DPV) as shown in FIG. 40. The oxidation onset for TPT-TT was determined to be 0.1 V vs. Fc/Fc⁺, equivalent to an IE of 5.22 eV (assuming Fc/Fc⁺ is −5.12 eV vs. vacuum). The IE and electron affinity estimates are described below. TPT-TT can be reversibly electrochemically doped in tetra-n-butylammonium hexafluorophosphate (TBAPF₆)/propylene carbonate as evidenced by the cyclic voltammetry and potential dependent UV-vis-NIR results in FIG. 40. The doped form of TPT-TT can also be accessed chemically in solution, evidenced by the bleaching of the π-π* transition and the growth of the carrier absorption just past 700 nm (FIG. 41), or via sequential doping of the thin films with iron(III) p-toluenesulfonate (ferric tosylate). Doping the as-cast samples with ferric tosylate results in an electrical conductivity of 18.5 S cm⁻¹ (±3.9 S cm⁻¹) and a Seebeck coefficient of 43.3 μV K⁻¹ (±4.8 μV K⁻¹). Thermal annealing at 250° C. followed by slow cooling of the samples prior to the doping, similar to the GIXD and DSC experiments, raises the conductivity ˜threefold to 54.9 S cm¹ (±4.6 S cm 1) and reduces the Seebeck coefficient to 26.9 μV K⁻¹ (+2.5 μV K⁻¹) as demonstrated in FIG. 42. The Seebeck coefficient serves as an indicator of the asymmetry of the electronic density of states around the Fermi level. A reduction in the Seebeck coefficient points to a decrease in energetic disorder in the material, and by extension, a potential reduction in the number of carriers trapped by energetic disorder. Increasing the doping level, on the other hand, would raise the Fermi level but also influence the symmetry of the density of states (72,73). Combined, our results suggest that thermal annealing creates a more ordered microstructure that reduces the energetic disorder in the film and/or enhances the efficiency of the chemical doping by generating more charge carriers in annealed films.

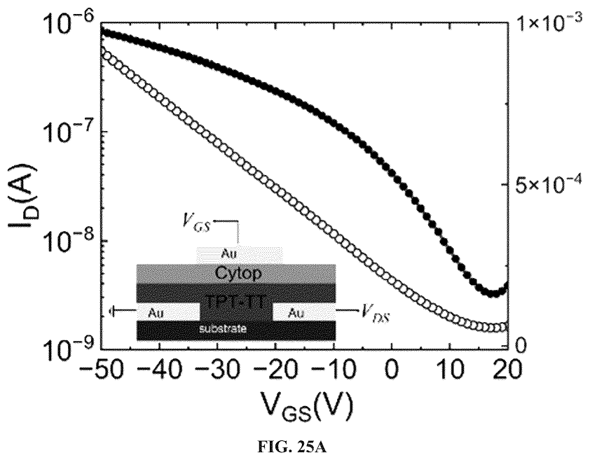

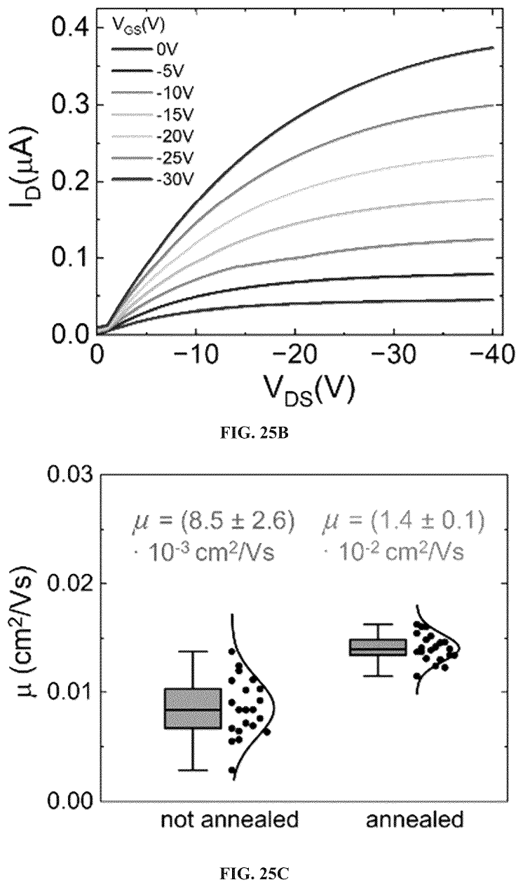

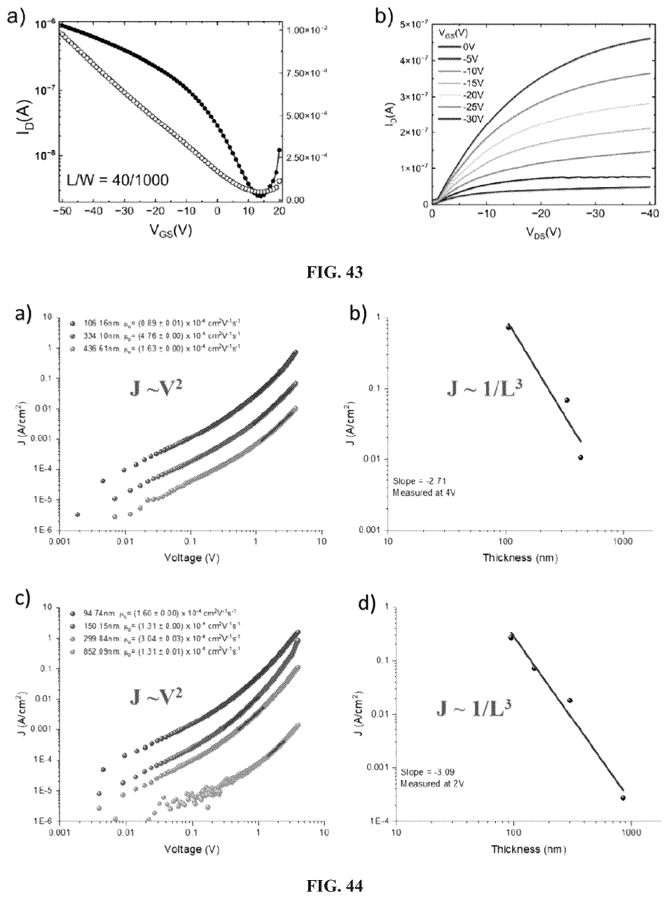

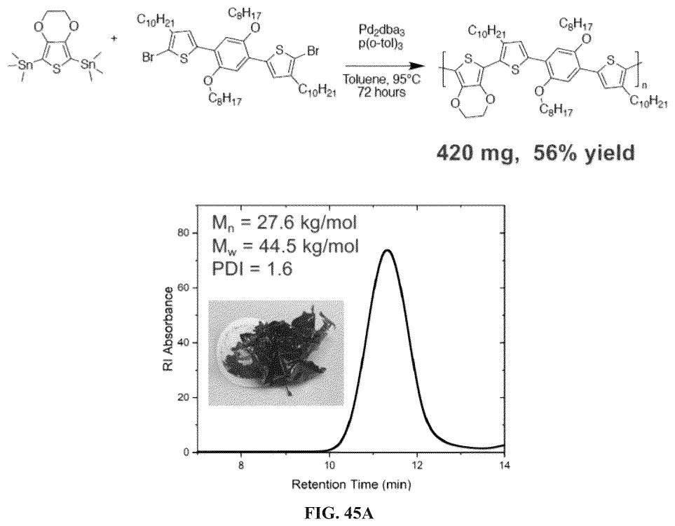

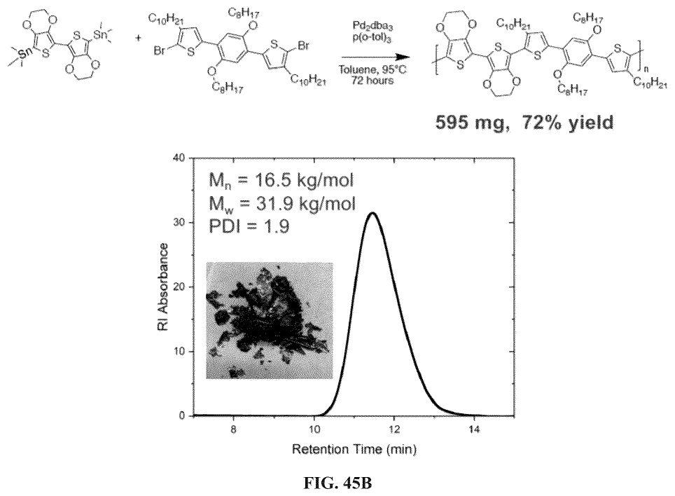

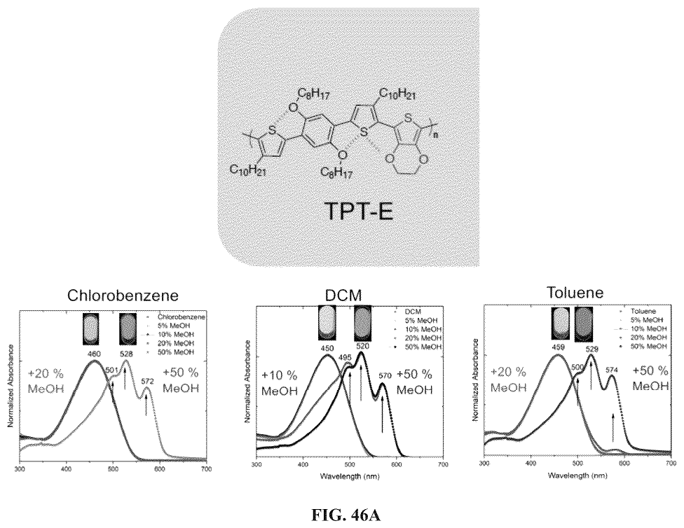

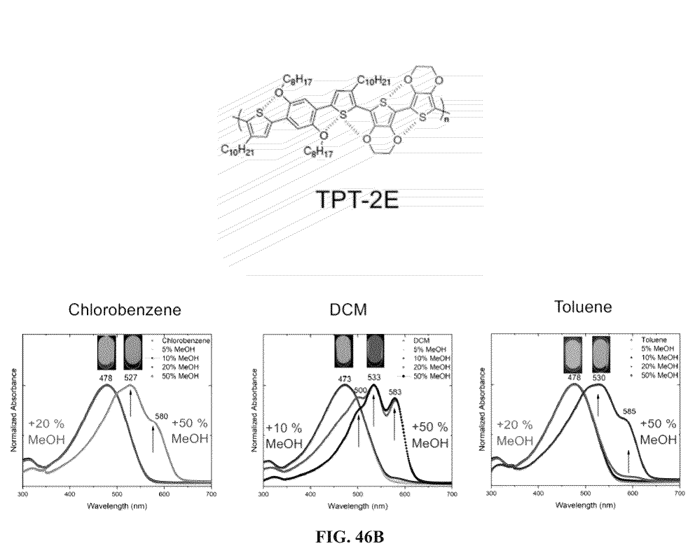

To further evaluate the charge transport properties of TPT-TT, we incorporated it as the active transport material in top-gate, bottom contacts organic field-effect transistors (OFETs) (74-76). The device fabrication details can be found below and the device structure is depicted in the inset in FIG. 25A. Along with the “as cast” reference films, a set of TPT-TT films have been annealed at 250° C. for 10 min in a glovebox, and then cooled slowly at 10° C. min⁻¹ to resemble the thermal history of the DSC, CPOM, and GIXD experiments. The devices were characterized in dark and under mild vacuum (77). FIGS. 25A-25B show typical transfer and output current-voltage characteristics for a device that has been annealed prior to characterization. The corresponding plots for an unannealed device are shown in FIG. 43. All OFETs exhibited p-type (hole) transport (i.e., enhancement in the drain current I_{D} for negative gate-source V_{GS} and drain-source V_{DS} voltages) (78). The device yield was 100% and the spread in electrical properties was very narrow. The OFETs exhibited almost ideal I-V curves, as can be observed in FIGS. 25A-25B, which is critical for the accuracy in the extraction of the figures of merit (79,78). The average charge carrier mobility for the OFETs made with as-cast TPT-TT films was μ=(8.5±2.6)×10⁻³ cm² V⁻¹ s⁻¹, while the average mobility extracted on devices fabricated using annealed films was μ=(1.4±0.1)×10⁻² cm² V⁻¹ s⁻¹. We complemented this in-plane mobility by space-charge-limited current (SCLC) out-of-plane measurements, the details of which are included below (FIG. 44). Similarly, SCLC mobilities (μ_{SCLC}=(2.43±0.01)×10⁻⁴ cm² V⁻¹ s⁻¹ for the as-spun samples and μ_{SCLC}=(1.82±0.02)×10⁻⁴ cm² V⁻¹ s⁻¹ for the annealed samples) are in the same range for the as-prepared and annealed films. The large observed variation in the measured mobility depending on the direction of charge transport, where we observe lower mobilities in out-of-plane SCLC regime compared to the OFET measured in-plane transport, is likely due to interfacial effects such as edge-on to face-on ratio throughout the film thickness (81), and the different morphological environments carriers experience along their transport pathways (82). While the increase in the average in-plane mobility for the OFETs after annealing is not large, note the narrower distribution in the annealed devices and the fact that the lowest mobility in the annealed devices is higher than the average value obtained in the samples that have not been annealed in devices with as-cast TPT-TT films (FIG. 25C). It is important to highlight the impact of this result in a more general context: the value of mobility, along with contact resistance and geometrical factors, impacts the operating frequency of the device, which, in turn, dictates the type of application that a certain FET technology can enable (83-85). For a large area transistor array, the mobilities of the lowest performing device determine the application that the ensemble can address. Thus, having a low variability in device properties and high yields is critical.

In summary, by incorporating temperature and solvent-dependent non-covalent intramolecular interactions into the structure of a symmetric π-conjugated unit (TPT), we have successfully induced liquid crystalline behavior in the TPT-TT polymer. The combination of π-π stacking and backbone planarity has resulted in a controllable solution aggregation and chain configuration properties. Through extensive GIXD studies, we identified: (i) a first LC phase, ranging from 218° C. to 107° C., as a nematic phase featuring preferential face-on π-π stacking and edge-on lamellar stacking exhibiting a large extent of disorder and broad orientation distribution; (ii) a second LC phase, from 107° C. to 48° C., as a smectic A phase featuring sharp, highly ordered out-of-plane lamellar stacking features and sharp tilted backbone stacking peaks; and (iii) a third LC phase with a transition at 48° C. whose structure remains unclear but resembles that of the solid state at ambient temperature. This liquid crystalline behavior leads to a significantly higher degree of order in the solid-state, achieved through thermal annealing. Due to the tendency of the backbone to planarize while having higher torsional freedom at elevated temperatures, the thermal annealing process promotes chain rearrangement into highly birefringent and ordered microstructures, which is reflected in the improved electrical conductivity of ferric tosylate-doped films and the observed low dispersity and high (100%) device yield in OFET devices. Moreover, we propose that the utilization of intra-chain interactions in combination with repeat unit symmetry is a powerful approach for promoting liquid crystallinity and designing highly ordered organic semiconductors.

### REFERENCES FOR EXAMPLE 2

1. A. Khasbaatar, Z. Xu, J.-H. Lee, G. Campillo-Alvarado, C. Hwang, B. N. Onusaitis and Y. Diao, Chem. Rev., 2023, 123, 8395-8487.
2. L. Ding, Z.-D. Yu, X.-Y. Wang, Z.-F. Yao, Y. Lu, C.-Y. Yang, J.-Y. Wang and J. Pei, Chem. Rev., 2023, 123, 7421-7497.
3. Z.-F. Yao, J.-Y. Wang and J. Pei, Prog. Polym. Sci., 2023, 136, 101626.
4. L. Zhang, K. Zhao, H. Li, T. Zhang, D. Liu and Y. Han, J. Polym. Sci., Part B: Polym. Phys., 2019, 57, 1572-1591.
5. R. Noriega, J. Rivnay, K. Vandewal, F. P. V. Koch, N. Stingelin, P. Smith, M. F. Toney and A. Salleo, Nat. Mater., 2013, 12, 1038-1044.
6. Z. Peng, L. Ye and H. Ade, Mater. Horiz., 2022, 9, 577-606.
7. N. A. Kukhta and C. K. Luscombe, Chem. Commun., 2022, 58, 6982-6997
8. K. Gu and Y.-L. Loo, J. Polym. Sci., Part B: Polym. Phys., 2019, 57, 1559-1571.
9. K. A. Mazzio, A. H. Rice, M. M. Durban and C. K. Luscombe, J. Phys. Chem. C, 2015, 119, 14911-14918.
10. N. Kleinhenz, N. Persson, Z. Xue, P. H. Chu, G. Wang, Z. Yuan, M. A. McBride, D. Choi, M. A. Grover and E. Reichmanis, Chem. Mater., 2016, 28, 3905-3913.
11. H. Lee, S. B. Lee, Y.-S. Kim, H. Kim, M.-J. Kim, T. W. Yoon, D. Lee, J. H. Cho, Y.-H. Kim and B. Kang, Chem. Eng. J., 2023, 468, 143654.
12. J. P. Ruiz, J. R. Dharia, J. R. Reynolds and L. J. Buckley, Macromolecules, 1992, 25, 849-860.
13. M. He, J. Li, A. Tandia, M. Sorensen, F. Zhang, H. H. Fong, V. A. Pozdin, D.-M. Smilgies and G. G. Malliaras, Chem. Mater., 2010, 22, 2770-2779.
14. T. Lei, Y. Cao, X. Zhou, Y. Peng, J. Bian and J. Pei, Chem. Mater., 2012, 24, 1762-1770.
15. C. Li, X. Gu, Z. Chen, X. Han, N. Yu, Y. Wei, J. Gao, H. Chen, M. Zhang, A. Wang, J. Zhang, Z. Wei, Q. Peng, Z. Tang, X. Hao, X. Zhang and H. Huang, J. Am. Chem. Soc., 2022, 144, 14731-14739.
16. K. Chung, D. S. Yang, W.-H. Sul, B.-G. Kim, J. Kim, G. Jang, M. S. Kwon, M. Barłóg, T. S. Lee, S.-Y. Park, M. Al-Hashimi and J. Kim, Macromolecules, 2019, 52, 6485-6494.
17. C. R. G. Grenier, W. Pisula, T. J. Joncheray, K. Mullen and J. R. Reynolds, Angew. Chem., Int. Ed., 2007, 46, 714-717.
18. J. Zhu, Y. Liu, S. Huang, S. Wen, X. Bao, M. Cai and J. Li, Phys. Chem. Chem. Phys., 2022,24, 17462-17470.
19. S. Ming, S. Zhen, H. Zhang, X. Han, Y. Zhang, J. Xu and J. Zhao, Eur. Polym. J., 2022, 163, 110938.
20. D. Liu, Y. Lei, X. Ji, Y. Wu, Y. Lin, Y. Wang, S. Zhang, Y. Zheng, Y. Chen, J.-C. Lai, D. Zhong, H.-W. Cheng, J. A. Chiong, X. Gu, S. Gam, Y. Yun, J. B. H. Tok and Z. Bao, Adv. Funct. Mater., 2022, 32, 2203527.
21. Y. Liu, Y. Liu and X. Zhan, Macromol. Chem. Phys., 2011, 212, 428-443.
22. C. Zhu, A. J. Kalin and L. Fang, Acc. Chem. Res., 2019, 52, 1089-1100.
23. A. Haque, K. M. Alenezi, M. S. Khan, W.-Y. Wong and P. R. Raithby, Chem. Soc. Rev., 2023, 52, 454-472.
24. K. S. Park, J. J. Kwok, R. Dilmurat, G. Qu, P. Kafle, X. Luo, S.-H. Jung, Y. Olivier, J.-K. Lee, J. Mei, D. Beljonne and Y. Diao, Sci. Adv., 2019, 5, eaaw7757.
25. S. Fratini, M. Nikolka, A. Salleo, G. Schweicher and H. Sirringhaus, Nat. Mater., 2020, 19, 491-502.
26. D. Venkateshvaran, M. Nikolka, A. Sadhanala, V. Lemaur, M. Zelazny, M. Kepa, M. Hurhangee, A. J. Kronemeijer, V. Pecunia, I. Nasrallah, I. Romanov, K. Broch, I. McCulloch, D. Emin, Y. Olivier, J. Cornil, D. Beljonne and H. Sirringhaus, Nature, 2014, 515, 384-388.
27. N. E. Jackson, B. M. Savoie, K. L. Kohlstedt, M. Olvera de la Cruz, G. C. Schatz, L. X. Chen and M. A. Ratner, J. Am. Chem. Soc., 2013, 135, 10475-10483.
28. Y. Cheng, Y. Qi, Y. Tang, C. Zheng, Y. Wan, W. Huang and R. Chen, J. Phys. Chem. Lett., 2016, 7, 3609-3615.
29. Z.-D. Yu, Y. Lu, J.-Y. Wang and J. Pei, Chem.-Eur. J., 2020, 26, 16194-16205.
30. B. Liu, D. Rocca, H. Yan and D. Pan, JACS Au, 2021, 1, 2182-2187.
31. T. Kharandiuk, E. J. Hussien, J. Cameron, R. Petrina, N. J. Findlay, R. Naumov, W. T. Klooster, S. J. Coles, Q. Ai, S. Goodlett, C. Risko and P. J. Skabara, Chem. Mater., 2019, 31, 7070-7079.
32. G. Conboy, H. J. Spencer, E. Angioni, A. L. Kanibolotsky, N. J. Findlay, S. J. Coles, C. Wilson, M. B. Pitak, C. Risko, V. Coropceanu, J.-L. Brddas and P. J. Skabara, Mater. Horiz., 2016, 3, 333-339.
33. Y. Zhang, C. Yu, T. Shan, Y. Chen, Y. Wang, M. Xie, T. Li, Z. Yang and H. Zhong, Cell Rep. Phys. Sci., 2022, 3, 100765.
34. S. Sabury, G. S. Collier, M. N. Ericson and S. M. Kilbey, Polym. Chem., 2020, 11, 820-829.
35. S. Sabury, B. LaRiviere, M. N. Ericson and S. M. Kilbey, II, ACS Appl. Polym. Mater., 2021, 3, 1012-102.
36. M. Liu, X. Han, H. Chen, Q. Peng and H. Huang, Nat. Commun., 2023, 14, 2500.
37. W. J. Mullin, S. A. Sharber and S. W. Thomas III, J. Polym. Sci., 2021, 59, 1643-1663.
38. P. J. W. Sommerville, A. H. Balzer, G. Lecroy, L. Guio, Y. Wang, J. W. Onorato, N. A. Kukhta, X. Gu, A. Salleo, N. Stingelin and C. K. Luscombe, ACS Polym. Au, 2023, 3, 59-69.
39. K. S. Ahn, H. Jo, J. B. Kim, I. Seo, H. H. Lee and D. R. Lee, ACS Appl. Mater. Interfaces, 2020, 12, 1142-1150.
40. Y. Yang, Z. Liu, G. Zhang, X. Zhang and D. Zhang, Adv. Mater., 2019, 31, 1903104.
41. I. McCulloch, M. Heeney, C. Bailey, K. Genevicius, I. MacDonald, M. Shkunov, D. Sparrowe, S. Tierney, R. Wagner, W. Zhang, M. L. Chabinyc, R. J. Kline, M. D. McGehee and M. F. Toney, Nat. Mater., 2006, 5, 328-333.
42. C. Poelking, E. Cho, A. Malafeev, V. Ivanov, K. Kremer, C. Risko, J.-L. Brddas and D. Andrienko, J. Phys. Chem. C, 2013, 117, 1633-1640.
43. V. Vijayakumar, E. Zaborova, L. Biniek, H. Zeng, L. Herrmann, A. Carvalho, O. Boyron, N. Leclerc and M. Brinkmann, ACS Appl. Mater. Interfaces, 2019, 11, 4942-4953.
44. P. Boufflet, Y. Han, Z. Fei, N. D. Treat, R. Li, D.-M. Smilgies, N. Stingelin, T. D. Anthopoulos and M. Heeney, Adv. Funct. Mater., 2015, 25, 7038-7048.
45. B. H. Hamadani, D. J. Gundlach, I. McCulloch and M. Heeney, Appl. Phys. Lett., 2007, 91, 243512.
46. K. Kang, S. Schott, D. Venkateshvaran, K. Broch, G. Schweicher, D. Harkin, C. Jellett, C. B. Nielsen, I. McCulloch and H. Sirringhaus, Mater. Today Phys., 2019, 8, 112-122.
47. W. A. Memon, Y. Zhang, J. Zhang, Y. Yan, Y. Wang and Z. Wei, Macromol. Rapid Commun., 2022, 43, 2100931.
48. T. Qu, Y. Li, L. Li, C. Zhang, X. Wang, W. Chen, G. Xue, E. Zhuravlev, S. Luo and D. Zhou, Macromolecules, 2023, 56, 6407-6418.
49. Y. Pan, J. Huang, D. Gao, Z. Chen, W. Zhang and G. Yu, Polym. Chem., 2021, 12, 2471-2480.
50. F. C. Spano and C. Silva, Annu. Rev. Phys. Chem., 2014, 65, 477-500.
51. N. Schopp, S. Sabury, T. Chaney, J. Zhang, H. Wakidi, B. M. Kim, R. Sankar, H. M. Luong, P. Therdkatanyuphong, V. V. Brus, S. Marder, M. F. Toney, J. R. Reynolds and T.-Q. Nguyen, ACS Energy Lett., 2023, 8, 3307-3313.
52. B. Xu, I. Pelse, S. Agarkar, S. Ito, J. Zhang, X. Yi, Y. Chujo, S. Marder, F. So and J. R. Reynolds, ACS Appl. Mater. Interfaces, 2018, 10, 44583-44588.
53. X. Yi, Z. Peng, B. Xu, D. Seyitliyev, C. H. Y. Ho, E. O. Danilov, T. Kim, J. R. Reynolds, A. Amassian, K. Gundogdu, H. Ade and F. So, Adv. Energy Mater., 2020, 10, 1902430.
54. S. Sabury, T. J. Adams, M. Kocherga, S. M. Kilbey and M. G. Walter, Polym. Chem., 2020, 11, 5735-5749.
55. Z. Du, H. Luong, S. Sabury, P. Therdkatanyuphong, S. Chae, C. Welton, A. Jones, J. Zhang, Z. Peng, Z. Zhu, S. Nanayakkara, V. Coropceanu, D. G. Choi, S. Xiao, A. Yi, H. J. Kim, J.-L. Bredas, H. Ade, G. N. Manjunatha Reddy, S. R. Marder, J. R. Reynolds and T.-Q. Nguyen, Mater. Horiz., 2023, 10, 5564-5576.
56. Z. Fei, P. Pattanasattayavong, Y. Han, B. C. Schroeder, F. Yan, R. J. Kline, T. D. Anthopoulos and M. Heeney, J. Am. Chem. Soc., 2014, 136, 15154-15157.
57. D. M. DeLongchamp, R. J. Kline, Y. Jung, D. S. Germack, E. K. Lin, A. J. Moad, L. J. Richter, M. F. Toney, M. Heeney and I. McCulloch, ACS Nano, 2009, 3, 780-787.
58. D. M. DeLongchamp, R. J. Kline, Y. Jung, E. K. Lin, D. A. Fischer, D. J. Gundlach, S. K. Cotts, A. J. Moad, L. J. Richter, M. F. Toney, M. Heeney and I. McCulloch, Macromolecules, 2008, 41, 5709-5715.
59. R. J. Mandle, S. J. Cowling and J. W. Goodby, Sci. Rep., 2017, 7, 13323.
60. R. J. Mandle, C. T. Archbold, J. P. Sarju, J. L. Andrews and J. W. Goodby, Sci. Rep., 2016, 6, 36682.
61. M. Bagnani, G. Nyström, C. De Michele and R. Mezzenga, ACS Nano, 2019, 13, 591-600.
62. Z. S. Davidson, Y. Huang, A. Gross, A. Martinez, T. Still, C. Zhou, P. J. Collings, R. D. Kamien and A. G. Yodh, Nat. Commun., 2017, 8, 15642.
63. G. Nyström, M. Arcari and R. Mezzenga, Nat. Nanotechnol., 2018, 13, 330-336.
64. P.-X. Wang and M. J. MacLachlan, Philos. Trans. R. Soc., A, 2017, 376, 20170042.
65. Y. Shi, F. G. Tournilhac and S. Kumar, Phys. Rev. E: Stat. Phys., Plasmas, Fluids, Relat. Interdiscip. Top., 1997, 55, 4382-4385.
66. S.-H. Yang and C.-S. Hsu, J. Polym. Sci., Part A: Polym. Chem., 2009, 47, 2713-2733.
67. M. Chang, J. Lee, N. Kleinhenz, B. Fu and E. Reichmanis, Adv. Funct. Mater., 2014, 24, 4457-4465.
68. Y. Minowa, Y. Yabuuchi, S. Nagano, S. Nagamatsu, A. Fujii and M. Ozaki, ACS Appl. Mater. Interfaces, 2022, 14, 50112-50119.
69. J. Kuebler, T. Loosbrock, J. Strzalka and L. Fernandez-Ballester, Macromolecules, 2023, 56, 3083-3094.
70. J. Nehring and A. Saupe, J. Chem. Soc., Faraday Trans. 2, 1972, 68, 1-15.
71. D. Trefz, Y. M. Gross, C. Dingler, R. Tkachov, A. Hamidi-Sakr, A. Kiriy, C. R. McNeill, M. Brinkmann and S. Ludwigs, Macromolecules, 2019, 52, 43-54.
72. K. Broch, D. Venkateshvaran, V. Lemaur, Y. Olivier, D. Beljonne, M. Zelazny, I. Nasrallah, D. J. Harkin, M. Statz, R. D. Pietro, A. J. Kronemeijer and H. Sirringhaus, Adv. Electron. Mater., 2017, 3, 1700225.
73. M. Lagdlund, R. Lazzaroni, S. Stafström, W. R. Salaneck and J. L. Brédas, Phys. Rev. Lett., 1989, 63, 1841-1844.
74. Z. A. Lamport, H. F. Haneef, S. Anand, M. Waldrip and O. D. Jurchescu, J. Appl. Phys., 2018, 124, 071101.
75. Z. A. Lamport, K. J. Barth, H. Lee, E. Gann, S. Engmann, H. Chen, M. Guthold, I. McCulloch, J. E. Anthony, L. J. Richter, D. M. DeLongchamp and O. D. Jurchescu, Nat. Commun., 2018, 9, 5130.
76. H. F. Iqbal, Q. Ai, K. J. Thorley, H. Chen, I. McCulloch, C. Risko, J. E. Anthony and O. D. Jurchescu, Nat. Commun., 2021, 12, 2352.
77. H. F. Iqbal, M. Waldrip, H. Chen, I. McCulloch and O. D. Jurchescu, Adv. Electron. Mater., 2021, 7, 2100393.
78. Y. Mei, D. Fogel, J. Chen, J. W. Ward, M. M. Payne, J. E. Anthony and O. D. Jurchescu, Org. Electron., 2017, 50, 100-105.
79. H. H. Choi, K. Cho, C. D. Frisbie, H. Sirringhaus and V. Podzorov, Nat. Mater., 2018, 17, 2-7.
80. E. G. Bittle, J. I. Basham, T. N. Jackson, O. D. Jurchescu and D. J. Gundlach, Nat. Commun., 2016, 7, 10908.
81. M. Saito, T. Koganezawa and I. Osaka, ACS Appl. Polym. Mater., 2019, 1, 1257-1262.
82. J. K. Wenderott, B. X. Dong and P. F. Green, Phys. Chem. Chem. Phys., 2021, 23, 27076-27102.
83. A. Perinot, M. Giorgio, V. Mattoli, D. Natali and M. Caironi, Adv. Sci., 2021, 8, 2001098.
84. H. Klauk, Adv. Electron. Mater., 2018, 4, 1700474.
85. M. Waldrip, O. D. Jurchescu, D. J. Gundlach and E. G. Bittle, Adv. Funct. Mater., 2020, 30, 1904576.

### Supplementary Experimental Details for Example 2

### Computational Methodology

### Ab-Initio Calculations

All DFT calculations were conducted at the M06-2X/6-311G** level of theory using the Gaussian 16 package (1). The dihedral angle between the studied groups were fixed from 0° to 360° at 10° intervals and geometry optimization was performed on all remaining degrees of freedom.

### Molecular Dynamics Simulations

All-atom (AA) molecular dynamics (MD) simulations were performed using the LAMMPS package (2) and the Optimized Potentials for Liquid Simulations-All Atom (OPLS-AA) force field (see FIG. 26 for dihedral scan of phenylenethienylene and thienylene-thienothiophene using optimized OPLS-AA Force Field) (3). Atom types and parameters were defined using the LigParGen web server (4). For the phenylene-thienylene and thienylene-thienothiophene dihedrals the force-field parameters were optimized based on results obtained from DFT calculations, the results are shown in FIG. 26 and optimized 13 coefficients for each dihedral are listed in Table 5. Atomic partial charges were determined by fitting the DFT-determined electrostatic potential (CM5) at the M06-2X/6-311G** level of theory (5). Long-range electrostatic interactions of charged particles were computed using the particle-particle-particle-mesh (PPPM) algorithm with a root-mean-accuracy of 10⁻⁵ (6). The Verlet integrator with a time step of 1 fs was used, and the Nose-Hoover thermostat/barostat was employed for temperature/pressure control. All related simulations were performed using either NVT (constant Number of particles, Volume, and Temperature) or NPT (constant number of molecules, pressure, and temperature) ensembles at pressure of 1 atm. A cutoff of 12 Å was applied for the summation of van der Waals interactions.

TABLE 5
OPLS-AA Optimized Force Field Parameters for
Phenylene-Thienylene and Thienylene-Thienothiophene
Dihedrals (13 Parameters Each)

| | Phenylene-Thienylene | Thienylene-Thienothiophene |
|---|---|---|
| A | 2.99494 | 1.5716 |
| B | 1.11645 | −0.14649 |
| C | −8.91671 | −4.97007 |
| D | −10.86306 | −13.663 |
| E | −1.88687 | −11.89236 |
| F | 48.13495 | 59.19301 |
| G | 4.03606 | 88.66136 |
| H | −70.29769 | −108.79631 |
| I | 4.69402 | −234.6759 |
| J | 41.2865 | 95.83407 |
| K | −11.05188 | 258.03093 |
| L | −7.25643 | −32.45405 |
| M | 3.40924 | −101.0592 |

To examine the aggregation dynamics of TPT-TT polymers in toluene and chlorobenzene, we conducted NPT simulations using trimer pair chains placed in both solvents at 15 mg/ml, across a temperature range of 270 to 330K. Simulations ran for 150 ns or until the centers of mass separated by 20 Å, a criterion established to indicate irreversible separation. This is to observe if the two trimers either separate or display a tendency to dissociate at different temperatures, thus offering insights into the solvation behavior of this polymer. FIG. 27 depicts the separation distance in relation to temperature for both solvents, showing a more pronounced separation in chlorobenzene, with distinct occurrence at room temperature. However, the chains in toluene maintained their association throughout the simulation, with only a slight increase in separation distance observed at 330 K. Since MD simulations operate on a different timescale compared to experiment, the lack of chain dissociation should not be viewed as an inherent characteristic of polymer aggregation, rather it serves as an indicator permitting a comparative assessment of polymer solubility within the investigated solvents.

### Synthesis of Intermediate Molecules and TPT-TT Monomer

All starting materials were purchased from a commercial supplier and were used without further purification. The synthesis pathway for TPT-TT (see FIG. 28), synthesis details of intermediate molecules, and the dibromo monomer are presented in the following section. Molecule 2 (2-(4-decylthiophen-2-yl)-4,4,5,5-tetramethyl-1,3,2-dioxaborolane) is synthesized following a procedure from literature (7).

¹H NMR and ¹³C NMR spectra for all monomers and molecular precursors were acquired on a Bruker Avance IIIHD 500 MHz or Bruker Avance IIIHD 700 MHz instruments using CDCl₃ as solvent; the residual CHCl₃ peak was used as a reference for all reported chemical shifts (1H: δ=7.26 ppm, 13C: δ=77.16 ppm). Mass spectrometry of small molecules was obtained by direct infusion atmospheric pressure chemical ionization (APCI) in positive mode using a Thermo Scientific Orbitrap ID-X Tribrid mass spectrometer. Gel permeation chromatography (GPC) was performed using a Tosoh EcoSEC high temperature GPC instrument with RI detector to determine the number average molecular weight (Mn), weight average molecular weight (Mw) and dispersity (Ð). Experiments were run using 1,2,4-trichlorobenzene (TCB) as the eluent at 140° C. at a flow rate of 1 mL/min on two 7.8 mm×30 cm, 13 μm TSK-Gel GMHHR-H(S) HT2 columns in series. The instrument was calibrated using polystyrene standards (1,390-1,214,000 g/mol) and the data were analyzed using 8321GPC-WS analysis software. The GPC samples were prepared by dissolving the polymers in TCB at a 1 mg/mL concentration and stirred at 120° C. for at least 3 hours before filtering through a 0.45 μm PTFE filter. Elemental analyses were conducted by Atlantic Microlab Inc. The MALDI-TOF mass spectra were performed with a Bruker Autoflex 111. The matrix substance used for all MALDI samples was trans-2-[3-(4-tert-Butylphenyl)-2-methyl-2-propenylidene]malononitrile (DCTB).

### Synthesis of 1,4-dibromo-2,5-bis(octyloxy)benzene (1)

Similar to a reported procedure (8), 50 mL of dry dimethylformamide (DMF) and 2,5-dibromohydroquinone (811 mg, 3.02 mmol) were added to a pre-dried 250 mL round bottom flask equipped with a stir bar. Then, 1.32 mL (7.55 mmol, 2.5 equiv.) of 1-bromooctane was added followed by the addition of excess potassium carbonate (1.24 g, 3 equiv.). the reaction mixture was left to stir overnight (16 hours) at 90° C. The solvent was removed under reduced pressure via rotary evaporation and the remaining contents were purified using silica gel plug (DCM as mobile solvent) followed by silica gel column chromatography with 2:1 ratio of hexane:dichloromethane as mobile phase to afford a white powder (1.4 g, 89%). ¹H NMR (700 MHz, CDCl₃), δ(ppm): 7.08 (s, 1H), 3.94 (t, J=6.5 Hz, 2H), 1.82-1.77 (m, 2H), 1.49-1.45 (m, 2H), 1.38-1.24 (m, 8H), 0.89 (t, J=7.0 Hz, 3H). ¹³C{¹H}NMR (176 MHz, CDCl₃), δ (ppm): 150.05, 118.44, 111.10, 70.29, 31.76, 29.23, 29.18, 29.08, 25.90, 22.63, 14.08. NMR spectra are consistent with a previous report (9).

### Synthesis of 5,5′-(2,5-bis(octyloxy)-1,4-phenylene)bis(3-decylthiophene) (3) (TPT core)

75 mL of dry dimethylformamide (DMF) and 1,4-dibromo-2,5-bis(octyloxy)benzene (1) (316 mg, 0.64 mmol) were added to a pre-dried 250 mL round bottom flask equipped with a stir bar. Then, 679 mg (1.86 mmol, 2.9 equiv.) of 2-(4-decylthiophen-2-yl)-4,4,5,5-tetramethyl-1,3,2-dioxaborolane (2) was added followed by the addition of 56 mg (0.1 equiv.) of [1,1′-bis(diphenylphosphino)ferrocene]palladium(II) dichloride dichloromethane adduct (Pd(dppf)Cl₂ DCM) as the catalyst. Excess potassium carbonate (309 mg, 3 equiv.) was added to the flask and the reaction mixture was left to stir overnight (16 hours) at 110° C. The solvent was removed under reduced pressure via rotary evaporation and the remaining contents were purified using silica gel plug (DCM as mobile solvent) followed by silica gel column chromatography with 4:1 ratio of hexane:dichloromethane as mobile phase to afford a white powder (350 mg, 70%). ¹H NMR (500 MHz, CDCl₃), δ(ppm): 7.38 (d, J=1.4 Hz, 1H), 7.21 (s, 1H), 6.92 (d, J=1.3 Hz, 1H), 4.07 (t, J=6.4 Hz, 2H), 2.63 (t, J=7.7 Hz, 2H), 1.93-1.86 (m, 2H), 1.70-1.62 (m, 2H), 1.57-1.51 (m, 2H), 1.39-1.24 (m, 22H), 0.91-0.86 (m, 6H). ¹³C{¹H}NMR (126 MHz, CDCl₃), δ (ppm): 149.44, 143.14, 139.10, 126.92, 123.14, 120.48, 112.85, 69.81, 32.07, 32.00, 30.79, 30.73, 29.81, 29.69, 29.63, 29.58, 29.51, 29.43, 26.47, 22.84, 14.27. HR-MS (APCI) m/z for C₅₀H₈₂O₂S₂ theoretical (M+H): 779.5828, found (M+H): 779.5814.

### Synthesis of 5,5′-(2,5-bis(octyloxy)-1,4-phenylene)bis(2-bromo-3-decylthiophene) (4) (dibromo TPT monomer)

320 mg of the molecule (3), 5,5′-(2,5-bis(octyloxy)-1,4-phenylene)bis(3-decylthiophene) (0.40 mmol) was added to a 250 mL round bottom flask, which had been pre-dried and equipped with a stir bar. Subsequently, 30 mL of chloroform was added. The resulting mixture was cooled to 0° C. using an ice bath. Next, 136 mg (0.78 mmol, 1.95 equiv.) of N-bromosuccinimide (NBS) was slowly added over a period of 10 minutes, and the reaction flask was covered from light. The reaction was then allowed to gradually return to room temperature by stopping the addition of ice to the bath. The product was extracted by using a mixture of dichloromethane (DCM) and water. The organic layer was dried with Na₂SO₄, filtered, and concentrated under reduced pressure. The product was purified by silica gel column chromatography with 5:1 ratio of hexane:dichloromethane as mobile phase to afford a pale yellow powder (280 mg, 75%). ¹H NMR (700 MHz, CDCl₃), δ(ppm): 7.20 (s, 1H), 7.12 (s, 1H), 4.07 (t, J=6.5 Hz, 2H), 2.60 (t, J=7.7 Hz, 2H), 1.93-1.88 (m, 2H), 1.71-1.63 (m, 2H), 1.57-1.52 (m, 2H), 1.39-1.24 (m, 22H), 0.91-0.86 (m, 6H). ¹³C{¹H}NMR (126 MHz, CDCl₃), δ (ppm): 149.25, 141.63, 138.44, 125.61, 122.57, 111.44, 110.17, 69.91, 32.07, 32.00, 30.04, 29.81, 29.79, 29.77, 29.64, 29.57, 29.54, 29.51, 29.50, 29.43, 26.46, 22.85, 14.28.

### Polymerization Procedure and General Characterizations

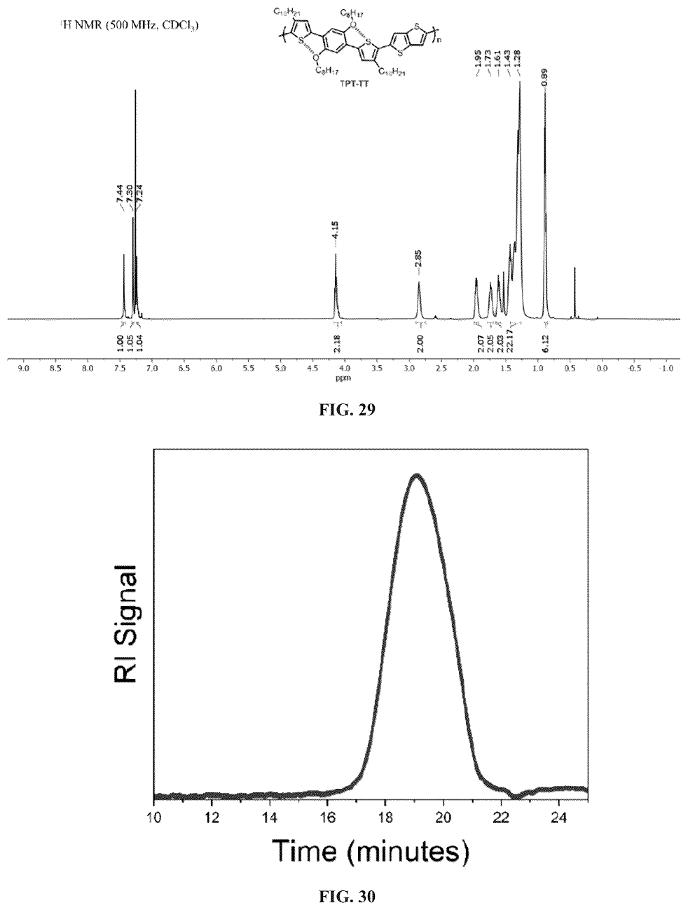

2,5-bis(trimethylstannyl)thieno[3,2-b]thiophene (200 mg, 0.43 mmol, 1 equiv.) and dibromo TPT monomer (402 mg, 0.43 mmol, 1 equiv.) were added to a freshly dried and cooled round bottom flask equipped with a stir bar. Subsequently, the flask was transferred to a glove box where dipalladium-tris(dibenzylideneacetone)chloroform complex (Pd₂(dba)₃ CHCl₃) was added as the catalyst (21 mg, 0.021 mmol, 0.05 equiv.), along with tris(o-tolyl)phosphine as the ligand (26 mg, 0.086 mmol, 0.2 equiv.), and 4 mL of toluene as the polymerization solvent. The reaction flask was then removed from the glove box and immersed in an oil bath set at 105° C., allowing it to stir for 16 hours at this temperature. After this time, a small amount of palladium scavenger (diethylammonium diethyldithiocarbamate) was added, and the temperature was lowered to 90° C. The solution was stirred for an additional hour at 90° C., followed by cooling to room temperature. The crude polymer was precipitated by adding it to stirring cold methanol. Further purification of the crude polymer was achieved through Soxhlet washing with methanol (24 hours), acetone (24 hours), and hexane (24 hours). The purified polymer was obtained via Soxhlet extraction using chloroform. Subsequently, the purified polymer was reprecipitated into cold methanol and collected as a red solid by vacuum filtration on a nylon membrane with a pore size of 45 μm. The number-average molecular weight was 24.6 kg/mol and the dispersity (Ð) was 1.93 with a monomodal distribution as measures by GPC (FIG. 30). MALDI-TOF was used to confirm equivalent incorporation of TPT and TT (FIGS. 31-32). ¹H NMR (500 MHz, CDCl₃), δ(ppm): 7.44 (s, 1H), 7.30 (s, 1H), 7.24 (s, 1H), 4.15 (m, 2H), 2.85 (m, 2H), 1.99-1.90 (m, 2H), 1.78-1.68 (m, 2H), 1.68-1.51 (m, 2H), 1.42-1.26 (m, 22H), 0.90-0.86 (m, 6H) (see FIG. 29). Anal. calcd. for C₅₆H₈₂O₂S₄: C (73.47%), S (14.01%), H (9.03%), O (3.50%); Found: C (72.84%), S (14.40%), H (9.02%).

### CPOM and GIXD Details

The films for CPOM measurements were prepared by spin-coating a 10 mg/mL chlorobenzene at 500 rpm onto a bare Si substrate. Subsequently, the films were relocated to a Linkam stage and the birefringence of the films was directly observed under CPOM throughout a thermal cycle. This involved heating the films above their melting temperature and subsequently cooling them down to room temperature at a rate of 10° C./min.

The GIXD measurements on the films were carried out at the Advanced Photon Source at the Argonne National Laboratory, specifically on beamline 8-ID-E. An incident beam energy of 10.92 keV was utilized to gather the diffraction data via a 2D detector (PILATUS 1M). Measurements were taken at two distinct positions, with a sample-to-detector distance set at 217 mm. The samples were scanned under a helium environment with an exposure time of 10 seconds and an incidence angle of 0.14°. The unit cell for the annealed sample was obtained by fitting the calculated peak positions of the reciprocal lattice {q_{xy}^{calc}, q_{z}^{calc}}={kb_{∥}*+lc_{∥}*, h|a*|+kb_{⊥}*+lc_{⊥}*} to experimentally measured peak positions. Here, the subscript II denotes the 2D in-plane reciprocal unit cell vectors (x,y-components), and ⊥ the respective out-of-plane components. The best fit unit cells are obtained by looking for reciprocal basis vectors a*, b*, c* for which the least square error

$${\sum }_{p e a k s} = {( \vert {q}_{xy}^{calc} − {q}_{xy}^{exp} \vert + \vert {q}_{z}^{c a l c} − {q}_{z}^{exp} \vert )}^{2}$$

is minimal. The fitted unit cell is reported with the b-c plane defined as parallel to the substrate.

### Electrochemical and Spectroelectrochemical Characterization

Differential pulse voltammetry (DPV) and cyclic voltammetry (CV) were performed on glassy carbon electrodes (area: 0.07 cm²). The glassy carbon electrodes were polished on polishing pads with Al₂O₃ slurries (1 μm, 0.1 μm, and 0.05 μm) to create a mirror finish and then rinsed with copious amounts of water. Films of TPT-TT were prepared via drop casting a one L aliquot of a 2 mg/mL TPT-TT in chloroform solution. The electrolyte used for all electrochemical experiments was 0.5 M tetrabutylammonium hexafluorophosphate in propylene carbonate (TBAPF₆/PC). TBAPF₆ was purchased from Acros Organics (98%) and purified via recrystallization from hot ethanol before use. The reference electrode was a Ag/Ag+ pseudoreference electrode (inner solution: 10 mM AgNO₃ in 0.5 M TBAPF₆ in acetonitrile, calibrated versus Fc/Fc+ that had an estimated E1/2 of 0.08 V). The counter electrode was a Pt flag. DPV and CV were recorded using an EG&G PAR 273A potentiostat/galvanostat under CorrWare control between −0.5 V and 0.8 V vs Ag/Ag+ (i.e., −0.58 and 0.72 V vs. Fc/Fc+). The scan rate used for the cyclic voltammetry was 20 mV/s whereas the DPV used a step size of 2 mV, step time of 0.08 seconds, and a pulse amplitude of 20 mV. All experiments were conducted under an argon blanket in degassed electrolyte solutions.

The onset of oxidation is found to be 0.1 V vs. Fc/Fc+, equivalent to an IE of 5.22 eV (assuming Fc/Fc+ is −5.12 eV vs. vacuum). Using the optical band gap of 2.07 eV (thin-film onset of absorption of 600 nm) and making the crude assumption of neglecting any exciton binding energy, the electron affinity is estimated to be 3.15 eV.

The spectroelectrochemistry and solution doping experiments were carried out with an Agilent Cary 5000 spectrophotometer and 1 cm path length quartz cuvettes. For the spectroelectrochemistry, TPT-TT films were spray-coated to an optical density of ˜1 from 4 mg/mL chroloform solutions with an Iwata Eclipse HP-BC airbrush spray gun with an optimized argon pressure of 20 psi onto ITO coated glass slides (8-12 ohm/sq, Delta Technologies). The ITO/glass electrodes had been cleaned by sonication in sodium dodecyl sulfate/H₂O, H₂O, toluene, and finally isopropanol (IPA) prior to use. The films were cycled 10 times from −0.58 to 0.72 V vs. Fc/Fc+ at 50 mV/s before recording a spectrum. The current and voltage were controlled and measured using an EG&G PAR 273A potentiostat/galvanostat. The films were held at the desired potential for 30 seconds before a spectrum was recorded. For the solution doping, TPT-TT was dissolved at a concentration of 0.05 mg/mL in chloroform and Fe(III)tos dissolved at 10 mg/mL in dry acetonitrile was titrated into the TPT-TT solution in aliquots of 0.25 equivalents.

The spectroelectrochemistry results (FIGS. 36A-36B) provide strong support for the DPV results where depletion of the π-π* band at 513 nm initiates at ˜0.1 V vs Fc/Fc+. Simultaneously, we observe the emergence of charge carrier bands at 749 nm and beyond 1600 nm. Notably, the high-energy charge carrier band reaches its peak intensity at ˜0.5 V vs Fc/Fc+, followed by a subsequent decrease in intensity accompanied by a blue-shift of the lower-energy charge carrier band. These findings collectively suggest the co-existence of radical cations and dications (polarons/bipolarons) in electrochemically doped TPT-TT.

### Electrical Conductivity, Seebeck Coefficient, and Doping Procedures

TPT-TT was dissolved in HPLC-grade chloroform with a stir bar at a concentration of 15 mg/mL. The solution was heated to 35° C. and stirred to aid in dissolution. Glass microscope slides were prescored and diced into strips about 1 cm wide and 5-6 cm long. Slides were cleaned by sonication in sodium dodecyl sulfate/DI water, followed by rinse with DI water, and sonication in IPA. The slides were wiped clean with a Kim-wipe and blown with compressed air to remove dust. The slides were loaded onto a shadow mask and four platinum contact pads in a van der Pauw geometry were deposited via magnetron sputtering using a custom-built sputtering chamber.

Films were formed by blade coating using a blade height of 175 μm (Zehntner Testing Instruments, ZAA 2300). Approximately 10-15 μL of 35° C. solution was put down as a thin line with a room temperature pipette and immediately blade coated with a speed of 50 mm/s. The polymer-coated strips were broken into approximately 1 cm×1 cm samples and vacuum dried at room temperature overnight. After vacuum drying, films were randomly selected and annealed in a glovebox by heating to 250° C. at 10° C./min, then slowly cooled to room temperature.

Dopant solutions were prepared by dissolving iron(III) p-toluenesulfonate (ferric tosylate) at a 50 mM concentration in acetonitrile. The estimated LUMO of the dopant lies deeper in energy than the HOMO of TPT-TT, at −5.25 eV vs. vacuum, enabling spontaneous electron transfer from the HOMO of TPT-TT to the LUMO of the dopant. The dopant solutions were prepared fresh and used immediately. 40 μL of dopant solution was drop-casted onto the TPT-TT films such that the area between the contact pads was completely covered. The films were then covered with a petri dish and left undisturbed for one minute. Then, the dopant solution was carefully pipetted off and the films were immersed in IPA to wash away any excess dopant salt. Films were then dried under vacuum for five minutes.

Electrical conductivity and Seebeck measurements were made with a custom setup. Micromanipulators with tungsten tips were used to gently pierce the film and make electrical contact to the bottom platinum contact pads. The in-plane electrical conductivity was acquired using the four-probe van der Pauw technique (Note: Pt contact pads were put under the polymer film, depositing Pt on the film surface resulted in incomplete doping under the contact pad and larger standard deviations). The Seebeck coefficient was measured by suspending the sample between two temperature-controlled Peltier units such that the center of the film between the contact pads was in the center of the gap (separated by 3 mm). The thermoelectric voltage was measured between two contact pads on separate stages using the probe tips, while the temperature of each pad was measured with a K-type thermocouple situated near the probe tips. A temperature difference of ΔT=10 K was applied to the film, and the resulting thermovoltage was measured. The Seebeck coefficient was calculated as the slope of the V vs ΔT plot. Voltage and temperature data were acquired using a Keithley 2700 DMM with a 7708 Mux card via a LabVIEW interface.

After transport measurements, the film thickness was measured with a Bruker DektakXT profilometer. A cut was made across the film in the direction of blade coating, then the average film thickness between the contact pads was measured in at least 2 different spots. Thicknesses were used to calculate corrected electrical conductivities.

### OFET Device Fabrication Details

Device fabrication started with cleaning the substrates in consecutive baths of warm acetone and warm IPA (85° C.), followed by UV-Ozone cleaning and a thorough DI water rinse and then drying under a stream of nitrogen. First, a bilayer of 3 nm Ti and 40 nm Au was deposited through a shadow mask to serve as source/drain contacts. The channel lengths L varied between 30 and 100 μm, while the channel widths W varied between 200 and 1000 μm. The contacts have been treated with pentafluoro benzene thiol (PFBT) to improve charge injection (10). Next, the TPT-TT layer was deposited by spin coating it from a 1 wt % solution in room temperature chlorobenzene, at a spinning speed of 1000 in a nitrogen glovebox. A set of films were annealed at 250° C. for 10 min in the same glovebox, then cooled slowly, while the other was used immediately after the spin-coating process. Without exposure to air, in the next step the polymer dielectric Cytop was spin coated at a spinning speed of 2000 rpm on top of TPT-TT, then cured in a vacuum oven at room temperature for about 12 h. The top gate electrode was then deposited through a shadow mask.

### Space-Charge-Limited Current (SCLC) Measurements

The out-of-plane mobility of the TPT-TT thin-film was assessed using a hole-only device configuration of ITO/MoO₃/polymer/MoO₃/Ag within the space-charge limited regime. For the hole-only device fabrication, the patterned ITO glass substrates were sequentially cleaned for 15 min in detergent solution (2% Mucasol (Schülke)), deionized water, acetone (Sigma-Aldrich, ≥99.5%), and IPA (Fisher Chemical). ITO substrates were dried with N₂ gun and then subjected to a UV ozone treatment for 10 minutes. A 10 nm layer of MoO₃ was evaporated at a pressure of 10-6 torr using a shadow mask to make 8 independent pixels on a single substrate; the active area of each pixel is 0.07 cm². The TPT-TT polymer dissolved in chlorobenzene (concentrations of 20-35 mg/ml) was deposited via static spin coating at a speed of 1000 rpm for 60 seconds with a ramp rate of 5 seconds. To explore the thickness dependence of J˜1/L3, four different concentrations of TPT-TT solutions were utilized: 20 mg/ml, 25 mg/ml, 30 mg/ml, and 35 mg/ml. Subsequently, TPT-TT thin films were annealed at 250° C. and then gradually cooled to room temperature. Then, a layer of MoO₃ (10 nm) followed by 100 nm of Ag were evaporated at a pressure of 10-6 torr on the active layer to obtain the completed devices. Fast-drying Ag paint (Ted Pella, 16040-30) was applied to the ITO electrodes to improve contacts with the device switch box. J-V characteristics were recorded with Keithley 2410 SourceMeter® SMU under dark conditions. Resulting J-V curves, mobility fits, and thickness dependence verifications for the annealed TPT-TT films as well as un-annealed films are shown below. TPT-TT solution preparation, spin-coating, evaporation, and J-V measurement were all done under Ar atmosphere inside the glovebox. Holemobility was extracted using the Mott-Gurney Law. The detailed equation for fitting and analysis process follows the previously reported literature (11).

### REFERENCES FOR SUPPLEMENTARY EXPERIMENTAL DETAILS FOR EXAMPLE 1

1. Gaussian 16, Revision C.01, M. J. Frisch, G. W. Trucks, H. B. Schlegel, G. E. Scuseria, M. A. Robb, J. R. Cheeseman, G. Scalmani, V. Barone, G. A. Petersson, H. Nakatsuji, X. Li, M. Caricato, A. V. Marenich, J. Bloino, B. G. Janesko, R. Gomperts, B. Mennucci, H. P. Hratchian, J. V. Ortiz, A. F. Izmaylov, J. L. Sonnenberg, D. Williams-Young, F. Ding, F. Lipparini, F. Egidi, J. Goings, B. Peng, A. Petrone, T. Henderson, D. Ranasinghe, V. G. Zakrzewski, J. Gao, N. Rega, G. Zheng, W. Liang, M. Hada, M. Ehara, K. Toyota, R. Fukuda, J. Hasegawa, M. Ishida, T. Nakajima, Y Honda, O. Kitao, H. Nakai, T. Vreven, K. Throssell, J. A. Montgomery, Jr., J. E. Peralta, F. Ogliaro, M. J. Bearpark, J. J. Heyd, E. N. Brothers, K. N. Kudin, V. N. Staroverov, T. A. Keith, R. Kobayashi, J. Normand, K. Raghavachari, A. P. Rendell, J. C. Burant, S. S. Iyengar, J. Tomasi, M. Cossi, J. M. Millam, M. Klene, C. Adamo, R. Cammi, J. W. Ochterski, R. L. Martin, K. Morokuma, O. Farkas, J. B. Foresman, and D. J. Fox, Gaussian, Inc., Wallingford CT, 2016.
2. Plimpton, S., Journal of computational physics, 1995, 117(1), pp. 1-19.
3. Jorgensen, W. L. and Tirado-Rives, J., Journal of the American Chemical Society, 1988, 110(6), pp. 1657-1666.
4. Dodda, L. S., Cabeza de Vaca, I., Tirado-Rives, J. and Jorgensen, W. L., Nucleic acids research, 2017, 45(W1), pp. W331-W336.
5. Marenich, A. V., Jerome, S. V, Cramer, C. J. and Truhlar, D. G., Journal of chemical theory and computation, 2012, 8(2), pp. 527-541.
6. Hockney, R. W. and Eastwood, J. W., 2021. Computer simulation using particles. crc Press.
7. Sato, S., Hashimoto, K. and Tajima, K., Synthetic metals, 2011, 161(13-14), pp. 1289-1298.
8. Wu, C. S. and Chen, Y. Journal of Polymer Science Part A: Polymer Chemistry, 2010, 48(24), pp. 5727-5736.
9. Feng, Y, Das, P. J., Young, R. M., Brown, P. J., Hornick, J. E., Weber, J. A., Seale, J. S., Stem, C. L., Wasielewski, M. R. and Stoddart, J. F., Journal of the American Chemical Society, 2022, 144(37), pp. 16841-16854.
10. Waldrip, M., Jurchescu, O. D., Gundlach, D. J. and Bittle, E. G., Advanced Functional Materials, 2020, 30(20), p. 1904576.
11. Advincula, A. A., Pelse, I. and Reynolds, J. R., Journal of Materials Chemistry C, 2020, 8(46), pp. 16452-16462.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A polymer or oligomer comprising a constitutional repeating unit of Formula I 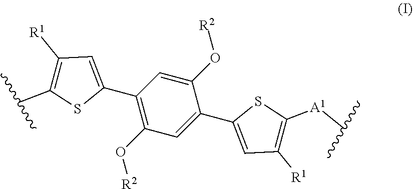
wherein:
R¹ and R² are independently selected at each occurrence from C₁-C₂₀ hydrocarbyl and —(CH₂CH₂O)ₘOCH₂CH₃, wherein m is an integer selected from 1 to 4, wherein each R¹ and R² may be optionally substituted with one or more groups independently selected from halogen, —NR^{a}R^{b}, —OR^{a}, and —P(═O)(OR^{a})(OR^{b});
R^{a} and R^{b} are independently selected at each occurrence from hydrogen and C₁-C₂₀ hydrocarbyl; and
A¹ is selected from:

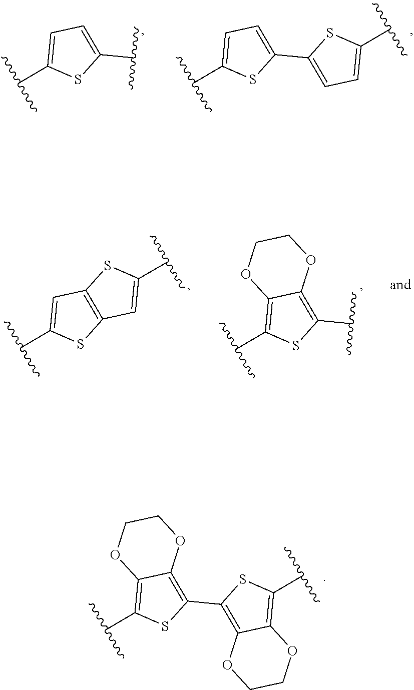 [SMILES: *c1ccc(*)s1 ; *c1ccc(-c2ccc(*)s2)s1 ; *c1cc2sc(*)cc2s1 ; *c1sc(*)c2c1OCCO2 ; *c1sc(-c2sc(*)c3c2OCCO3)c2c1OCCO2]

2. The polymer or oligomer of claim 1, wherein A¹ is

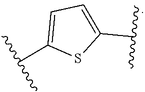 [SMILES: *c1ccc(*)s1]

3. The polymer or oligomer of claim 1, wherein A¹ is

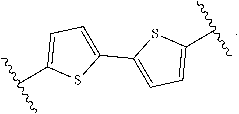 [SMILES: *c1ccc(-c2ccc(*)s2)s1]

4. The polymer or oligomer of claim 1, wherein A¹ is

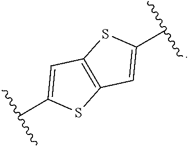 [SMILES: *c1cc2sc(*)cc2s1]

5. The polymer or oligomer of claim 1, wherein A¹ is

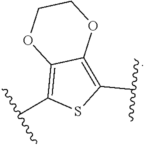 [SMILES: *c1sc(*)c2c1OCCO2]

6. The polymer or oligomer of claim 1, wherein A¹ is

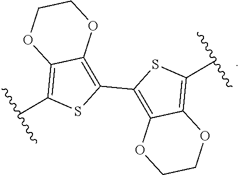 [SMILES: *c1sc(-c2sc(*)c3c2OCCO3)c2c1OCCO2]

7. The polymer or oligomer of claim 1, wherein R¹ is independently selected at each occurrence from C₁-C₂₀ alkyl.

8. The polymer of oligomer of claim 1, wherein R¹ is independently selected at each occurrence from C₆-C₁₀ alkyl.

9. The polymer of oligomer of claim 1, wherein R¹ is independently selected at each occurrence from —(CH₂)₅CH₃ and —(CH₂)₉CH₃.

10. The polymer or oligomer of claim 1, wherein R² is independently selected at each occurrence from C₁-C₂₀ alkyl.

11. The polymer or oligomer of claim 1, wherein R² is independently selected at each occurrence from C₅-C₈ alkyl.

12. The polymer or oligomer of claim 1, wherein R² is independently selected at each occurrence from —(CH₂)₇CH₃ or —CH₂(CHCH₃)CH₂CH₃.

13. The polymer or oligomer of claim 1, wherein the polymer or oligomer comprises a constitutional repeating unit selected from

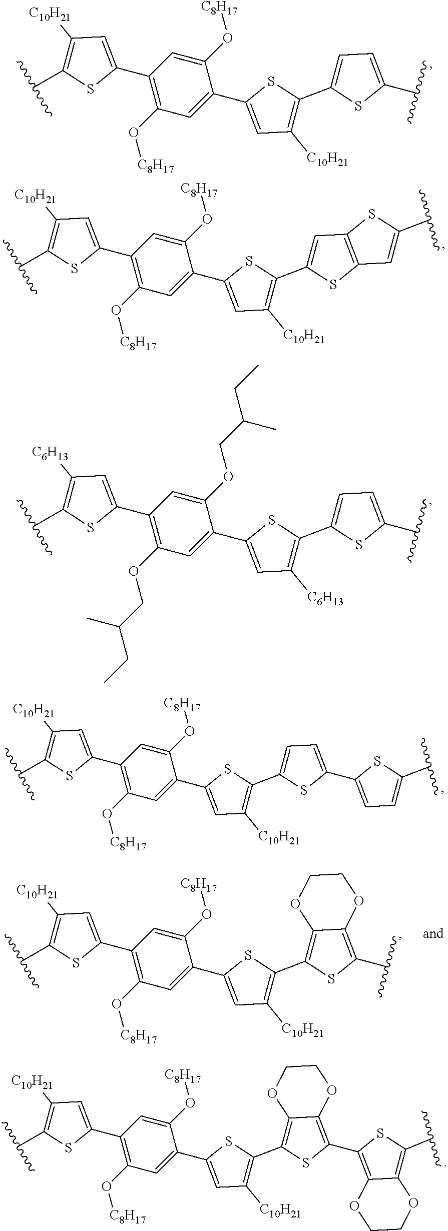

14. A composition comprising a polymer or oligomer of claim 1.

15. The composition of claim 14, further comprising one or more additives.

16. The composition of claim 15, wherein the one or more additives are selected from lithium bis(trifluoromethane)sulfonimide (Li-TFSI), 4-tert-butylpyridine (tBP), or combinations thereof.

17. A device comprising a polymer or oligomer of claim 1.

18. The device of claim 17, wherein the device is a photovoltaic device, a light-emitting diode, or a field-effect transistor.

19. A photovoltaic device comprising:
At least one perovskite material layer; and
at least one hole transport layer comprising a polymer or oligomer of claim 1.

20. The photovoltaic device of claim 19, further comprising at least one electron transport layer.
